(19) United States
(12) Patent Application Publication
(10) Pub. No.: US 2014/0113876 A1
(43) Pub. Date: Apr. 24, 2014

(54) BIOMARKERS FOR ASSESSING TREATMENT OF SIALIC ACID DEFICIENCY DISEASES AND CONDITIONS

(71) Applicant: ULTRAGENYX PHARMACEUTICAL INC., Novato, CA (US)

(72) Inventors: Yiumo CHAN, Novato, CA (US); Steve JUNGLES, Novato, CA (US); Emil KAKKIS, Novato, CA (US)

(73) Assignee: ULTRAGENYX PHARMACEUTICAL INC., Novato, CA (US)

(21) Appl. No.: 14/032,882

(22) Filed: Sep. 20, 2013

Related U.S. Application Data
(60) Provisional application No. 61/704,373, filed on Sep. 21, 2012.
(60) Provisional application No. 61/779,929, filed on Mar. 13, 2013.

(51) Int. Cl. C12Q 1/68; G01N 33/68

(52) U.S. Cl. CPC C12Q 1/6883; G01N 33/6893
USPC 514/23; 436/501; 506/16; 506/9

(57) ABSTRACT

The present invention relates to methods of monitoring and assessing sialic acid deficiency treatment as well as to methods of predicting/determining responsiveness to treatment for a sialic acid deficiency using biomarkers. Sialic acid deficiencies include for example Hereditary Inclusion Body Myopathy (HIBM).

### CROSS-REFERENCE TO RELATED APPLICATIONS

This application claims the benefit of U.S. Provisional Patent Application Ser. Nos. 61/704,373 filed on Sep. 21, 2012 and 61/779,929 filed on Mar. 13, 2013, each of which is herein incorporated by reference in its entirety.

### TECHNICAL FIELD

The present invention relates to biomarkers for determining responsiveness and monitoring the treatment of sialic acid deficiency diseases and conditions, e.g., Hereditary Inclusion Body Myopathy (HIBM).

### BACKGROUND

Sialic acid is the only sugar that contains a net negative charge and is typically found on terminating branches of N-glycans, O-glycans, and glycosphingolipids (gangliosides) (and occasionally capping side chains of GPI anchors). The sialic acid modification of cell surface molecules is crucial for many biological phenomena including protein structure and stability, regulation of cell adhesion, and signal transduction. Sialic acid deficiency disorders such as Hereditary Inclusion Body Myopathy (HIBM or HIBM type 2), Nonaka myopathy, and Distal Myopathy with Rimmed Vacuoles (DMRV) are clinical diseases resulting from a reduction in sialic acid production.

HIBM is a rare autosomal recessive neuromuscular disorder caused by a specific biosynthetic defect in the sialic acid synthesis pathway. Eisenberg et al., Nat. Genet. 29:83-87 (2001). The disease manifests between the ages of 20 to 40 with foot drop and slowly progressive muscle weakness and atrophy. Patients may suffer difficulties walking with foot drop, gripping and using their hands, and normal body functions like swallowing. Histologically, it is associated with muscle fiber degeneration and formation of vacuoles containing 15-18 nm tubulofilaments that immunoreactive like β-amyloid, ubiquitin, prion protein and other amyloid-related proteins. Askanas et al., Curr Opin Rheumatol. 10:530-542 (1998). Both the progressive weakness and histological changes initially spare the quadriceps and certain other muscles of the face. However, the disease is relentlessly progressive with patients becoming incapacitated and wheelchair-confined within one to two decades. There are no treatments currently available. Other causative mutations were identified for HIBM in the gene GNE, which encodes the bifunctional enzyme UDP-N-acetylglucosamine-2-epimerase/N-acetylmannosamine kinase (GNE/MNK). Studies of an Iranian-Jewish genetic isolate mapped the mutation associated with HIBM to chromosome 9p12-13. Argov et al., Neurology 60:1519-1523 (2003). Eisenberg et al., Nat. Genet. 29:83-87 (2001). DMRV is a Japanese variant, allelic to HIBM. Nishino et al., Neurology 59:1689-1693 (2002).

The current assessment of HIBM patients requires the use of a muscle biopsy and the assessment of sialylation of muscle bound glycoproteins such as PSA-NCAM. Ricci et al., Neurology, 66(5), 755-8 (2006); Broccolini et al., Neurology 75 265-272 (2010); Tajima et al., The American Journal of Pathology, 166(4) 1121-1130 (2005); Nemunaitis et al., J Gene Med, 12(5) 403-12 (2010). Muscle biopsies cannot be assessed regularly, are difficult to quantify and cannot be used reliably for regular management or drug development studies. Assessment can also be done genotypically.

Given the problems associated with current methods for diagnosing HIBM and determining responsiveness to and/or monitoring treatment of HIBM patients, there is a need for methods which allow for diagnosing and monitoring treatment of HIBM patients.

### BRIEF SUMMARY OF THE INVENTION

Embodiments of the present invention include methods for monitoring responsiveness or efficacy of a treatment with a sialic acid deficiency treatment in a subject suffering from a sialic acid deficiency. The methods comprise detecting the level of one or more sialic acid replacement therapy (SAT) biomarkers in a biological sample from the subject treated for a sialic acid deficiency, wherein an increase or decrease in the level of one or more SAT biomarkers indicates efficacy of the treatment with the sialic acid deficiency treatment. In some embodiments, the method is conducted within a short period of time after the treatment starts. In some embodiments, within the short period of time, there is no obvious alteration of symptoms of the diseases due to the treatment yet. For example, the detecting step is conducted right after at least one administration of the sialic acid deficiency treatment. In some embodiments, the presence or absence of a normalization or stabilization in the level of one or more SAT biomarkers toward a predetermined standard level indicates efficacy of treatment with the sialic acid deficiency treatment a short period of time after the treatment starts.

Embodiments of the present invention also include methods for determining the treatment regimen for treating a sialic acid deficiency comprising detecting the level of one or more SAT biomarkers in a biological sample from a subject treated for a sialic acid deficiency and determining a treatment regimen based on an increase or decrease in the level of one or more SAT biomarkers in the biological sample. In some embodiments, the method is conducted before the treatment. In some embodiments, the treatment is adopted if an increase or decrease in the baseline level of one or more SAT biomarkers in the biological sample is present compared to a predetermined standard level. In some embodiments, the method is conducted within a short period of time after the treatment starts. In some embodiments, within the short period of time, there is no obvious alteration of symptoms of the diseases due to the treatment yet. For example, the detecting step is conducted right after at least one administration of the sialic acid deficiency treatment. In some embodiments, the determination is based on the presence or absence of a normalization or stabilization in the level of one or more SAT biomarkers toward a predetermined standard level in the biological sample a short period of time after the treatment starts.

Embodiments of the present invention also include methods for predicting the treatment efficacy of a sialic acid deficiency treatment, comprising detecting the level of one or more SAT biomarkers in a biological sample from a subject, wherein an increase or decrease in the level of one or more SAT biomarkers compared to a predetermined standard level is predictive of the treatment efficacy of the sialic acid deficiency treatment. In some embodiments, the method is conducted before the treatment. In some embodiments, the treatment is predicted to be effective if an increase or decrease in the baseline level of one or more SAT biomarkers in the biological sample is present compared to a predetermined standard level. In some embodiments, the method is conducted within a short period of time after the treatment starts. In some embodiments, within the short period of time, there is no obvious alteration of symptoms of the diseases due to the treatment yet. For example, the detecting step is conducted right after at least one administration of the sialic acid deficiency treatment. In some embodiments, the presence or absence of a normalization or stabilization in the level of one or more SAT biomarkers toward a predetermined standard level is predictive of the treatment efficacy of the sialic acid deficiency treatment.

Embodiments of the present invention also include methods for determining whether a subject with a sialic acid deficiency is suitable for a sialic acid deficiency treatment comprising detecting the level of one or more SAT biomarkers in a sample from the subject, wherein an increase or decrease in the level of one or more SAT biomarkers compared to a predetermined standard level indicates a subject is suitable for a sialic acid deficiency treatment. In some embodiments, the method is conducted before the treatment. In some embodiments, the subject is predicted to be suitable for the treatment if an increase or decrease in the baseline level of one or more SAT biomarkers in the biological sample is present compared to a predetermined standard level. In some embodiments, the method is conducted within a short period of time after the treatment starts. In some embodiments, within the short period of time, there is no obvious alteration of symptoms of the diseases due to the treatment yet. For example, the detecting step is conducted right after at least one administration of the sialic acid deficiency treatment. In some embodiments, the presence or absence of a normalization or stabilization in the level of one or more SAT biomarkers toward a predetermined standard level indicates a subject is suitable for the sialic acid deficiency treatment.

Embodiments of the present invention provide methods for treating a subject with a sialic acid deficiency. These methods comprise detecting the level of one or more SAT biomarkers in a biological sample from the subject and administering a sialic acid deficiency treatment to the subject if the level of one or more SAT biomarkers is increased or decreased compared to a predetermined standard level. In some embodiments, the detecting step is conducted before the treatment. In some embodiments, a sialic acid deficiency treatment is administered to the subject if the baseline level of one or more SAT biomarkers is increased or decreased compared to a predetermined standard level. In some embodiments, the method is conducted within a short period of time after the treatment starts. In some embodiments, within the short period of time, there is no obvious alteration of symptoms of the diseases due to the treatment yet. For example, the detecting step is conducted right after at least one administration of the sialic acid deficiency treatment. In some embodiments, a sialic acid deficiency treatment is continued to be administered to the patient if there is a normalization or stabilization in the level of one or more SAT biomarkers toward a predetermined standard level.

Embodiments of the present invention provide methods for treating a subject with a sialic acid deficiency. These methods comprise receiving information on the level of one or more SAT biomarkers in a biological sample from the subject, and administering a sialic acid deficiency treatment to the subject if the level of one or more SAT biomarkers is increased or decreased compared to a predetermined standard level. In some embodiments, the information is collected before the treatment. In some embodiments, the sialic acid deficiency treatment is administered to the subject if an increase or decrease in the baseline level of one or more SAT biomarkers in the biological sample is present compared to a predetermined standard level. In some embodiments, the information is collected within a short period of time after the treatment starts. In some embodiments, within the short period of time, there is no obvious alteration of symptoms of the diseases due to the treatment yet. For example, the detecting step is conducted right after at least one administration of the sialic acid deficiency treatment. In some embodiments, a sialic acid deficiency treatment is continuously administered to the patient if there is a normalization or stabilization in the level of one or more SAT biomarkers toward a predetermined standard level.

Embodiments of the present invention provide methods for providing data comprising detecting the level of one or more SAT biomarkers in a sample from a subject and providing the information regarding the level of one or more biomarkers to a healthcare provider for diagnosis or treatment of the subject. In some embodiments, the data is collected before the treatment. In some embodiments, the data is collected within a short period of time after the treatment starts. For example, the data is collected right after at least one administration of the sialic acid deficiency treatment.

Embodiments of the present invention provide methods of providing useful information for predicting or determining the treatment efficacy of a sialic acid deficiency treatment comprising determining the level of one or more SAT biomarkers from a biological sample of a subject and providing the level of one or more SAT biomarkers to an entity that provides a prediction or determination of the treatment efficacy based on an increase or decrease in the level of one or more of the SAT biomarkers in a subject. In some embodiments, the information is collected before the treatment. In some embodiments, the determination is based on an increase or decrease in the baseline level of one or more SAT biomarkers in the biological sample compared to a predetermined standard level. In some embodiments, the information is collected within a short period of time after the treatment starts. In some embodiments, within the short period of time, there is no obvious alteration of symptoms of the diseases due to the treatment yet. In some embodiments, the determination is based on the presence or absence of a normalization or stabilization in the level of one or more SAT biomarkers toward a predetermined standard level in the biological sample.

Embodiments of the present invention provide a combination of tests useful for predicting or determining the treatment efficacy of sialic acid deficiency treatment comprising a first test for detecting the level of one SAT biomarker from a biological sample from a subject and a second test for detecting the level of a second SAT biomarker from a biological sample, wherein the first SAT biomarker is different from the second SAT biomarker.

Embodiments of the present invention provide for kits comprising reagents for detecting the level of one or more SAT biomarkers in a biological sample and an instruction for using the SAT biomarker according to any of the methods described herein.

Embodiments of the present invention provide for a collection of level of a panel of SAT biomarkers. In some embodiments, the SAT biomarkers comprise at least two or more SAT biomarkers of the present invention. In some embodiments, the SAT biomarkers are selected from those listed in Tables 2-14. In some embodiments, the SAT biomarkers are selected from AgRP, AR, BDNF, CD40-L, CgA, Cortisol, CK-MB, EGF, ENA-78, FGF-4, IGFBP-6, IL-3, IL-5, IL-7, IL-8, Kallikrein 5, LAP TGF-b1, M-CSF, MIP-3 alpha, MMP-1, MMP-3, MMP-9, MPO, Myoglobin, NT proBNP, NSE, Nr-CAM, PAI-1, PDGF-BB, S100-A4, S100-A6, ErbB3, SGOT, RANTES, Thrombospondin-1, TG and VEGF-C. In some embodiments, SAT biomarkers can include but are not limited to Chromogranin-A (CgA), Epithelial-Derived Neutrophil-Activating Protein 78 (ENA-78) Lactoylglutathione lyase (LGL), Latency-Associated Peptide of Transforming Growth Factor beta 1 (LAP TGF-b1), Macrophage Colony-Stimulating Factor 1 (M-CSF), Matrix Metalloproteinase-9, total (MMP-9, total), Myoglobin, Neuronal Cell Adhesion Molecule (Nr-CAM), Plasminogen Activator Inhibitor 1 (PAI-1), Receptor tyrosine-protein kinase erbB-3 (ErbB3), and Vascular Endothelial Growth Factor C (VEGF-C).

Embodiments of the present invention provide for an array comprising probes for detection of at least two or more biomarkers. In some embodiments, the SAT biomarkers comprise at least two or more SAT biomarkers of the present invention. In some embodiments, the SAT biomarkers are selected from those listed in Tables 2-14. In some embodiments, the SAT biomarkers are selected from AgRP, AR, BDNF, CD40-L, CgA, Cortisol, CK-MB, EGF, ENA-78, FGF-4, IGFBP-6, IL-3, IL-5, IL-7, IL-8, Kallikrein 5, LAP TGF-b1, M-CSF, MIP-3 alpha, MMP-1, MMP-3, MMP-9, MPO, Myoglobin, NT proBNP, NSE, Nr-CAM, PAI-1, PDGF-BB, S100-A4, S100-A6, ErbB3, SGOT, RANTES, Thrombospondin-1, TG and VEGF-C. In some embodiments, the array is a microarray. In some embodiments, SAT biomarkers can include but are not limited to Chromogranin-A (CgA), Epithelial-Derived Neutrophil-Activating Protein 78 (ENA-78) Lactoylglutathione lyase (LGL), Latency-Associated Peptide of Transforming Growth Factor beta 1 (LAP TGF-b1), Macrophage Colony-Stimulating Factor 1 (M-CSF), Matrix Metalloproteinase-9, total (MMP-9, total), Myoglobin, Neuronal Cell Adhesion Molecule (Nr-CAM), Plasminogen Activator Inhibitor 1 (PAI-1), Receptor tyrosine-protein kinase erbB-3 (ErbB3), and Vascular Endothelial Growth Factor C (VEGF-C).

### BRIEF DESCRIPTION OF THE DRAWINGS

FIG. 1 provides a diagram of intracellular sialic acid metabolism.

FIG. 2. LAP TGF-b1 as an example of biomarker showing trend of normalization when the baseline level in HIBM patients was higher than normal controls. Serum level of the analyte at week 0 and week 24 from individual patient (black lines) within each dosage group was plotted. Levels of LAP TGF-b1 in 6 g/day dosage group declined at a faster rate (average delta, bolded line) towards normal level (green line) compared to placebo. FIG. 2A depicts change in serum biomarker levels in HIBM patients of placebo group between week 0 and week 24. FIG. 2B depicts change in serum biomarker levels in HIBM patients of 6 g/day group between week 0 and week 24.

FIG. 3. NCAM as an example of biomarker showing trend of normalization when the baseline level in HIBM patients was lower than normal controls. Serum level of the analyte at week 0 and week 24 from individual patient (black lines) within each dosage group was plotted. Levels of NCAM in 6 g/day dosage group increased at a faster rate (average delta, bolded line) towards normal level (green line) compared to placebo. FIG. 3A depicts change in serum biomarker levels in HIBM patients of placebo group between week 0 and week 24. FIG. 3B depicts change in serum biomarker levels in HIBM patients of 6 g/day group between week 0 and week 24.

FIGS. 4A-4D. Delta (A) of serum biomarkers between week 0 and week 24 in each dosage group. For analytes with baseline levels in HIBM patients higher than normal controls (marked as RED), the slope of delta always displayed a downward trend from placebo to 6 g/day group (x-axis), indicating a more favorable change in the 6 g/day group moving toward normal levels. In contrast, for analytes with baseline levels lower than normal controls (marked as BLUE), the slope of delta always displayed a upward trend.

### DETAILED DESCRIPTION OF THE INVENTION

As used herein, the following terms shall have the following meanings:

The verb “comprise” as is used in this description and in the claims and its conjugations are used in its non-limiting sense to mean that items following the word are included, but items not specifically mentioned are not excluded.

The term “a” or “an” refers to one or more of that entity; for example, “a gene” refers to one or more genes or at least one gene. As such, the terms “a” (or “an”), “one or more” and “at least one” are used interchangeably herein. In addition, reference to “an element” by the indefinite article “a” or “an” does not exclude the possibility that more than one of the elements are present, unless the context clearly requires that there is one and only one of the elements.

The invention provides isolated, chimeric, recombinant or synthetic polynucleotide sequences. As used herein, the terms “polynucleotide”, “polynucleotide sequence”, “nucleic acid sequence”, “nucleic acid fragment”, and “isolated nucleic acid fragment” are used interchangeably herein and encompass DNA, RNA, cDNA, whether single stranded or double stranded, as well as chemical modifications thereof. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. Nucleotides (usually found in their 5′-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide. In some embodiments, the isolated, chimeric, recombinant or synthetic polynucleotide sequences are derived from gene markers of the present invention.

Single letter amino acid abbreviations used herein have their standard meaning in the art, and all peptide sequences described herein are written according to convention, with the N-terminal end to the left and the C-terminal end to the right.

The invention provides probes and primers that are derived from the nucleic acid sequences of the biomarker genes. The term “probe” as used herein refers to an oligonucleotide which is capable of specific annealing to the amplification target. The term “primer” as used herein refers to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer must be sufficiently long to prime the synthesis of extension products in the presence of the agent for polymerization. The exact lengths of the primers will depend on many factors, including temperature and composition (A/T vs. G/C content) of primer. A pair of bi-directional primers consists of one forward and one reverse primer as commonly used in the art of DNA amplification such as in PCR amplification.

The terms “array” or “matrix” refer to an arrangement of addressable locations or “addresses” on a device. The locations can be arranged in two-dimensional arrays, three-dimensional arrays, or other matrix formats. The number of locations may range from several to at least hundreds of thousands. Most importantly, each location represents a totally independent reaction site. A “nucleic acid array” refers to an array containing nucleic acid probes, such as oligonucleotides or larger portions of genes. The nucleic acid on the array is preferably single-stranded. Arrays wherein the probes are oligonucleotides are referred to as “oligonucleotide arrays” or “oligonucleotide chips.” A “microarray,” also referred to herein as a “biochip” or “biological chip,” is an array of regions having a density of discrete regions of at least about 100/cm², and preferably at least about 1000/cm². The regions in a microarray have typical dimensions, for example, diameters, in the range of between about 10-250 μm, and are separated from other regions in the array by about the same distance. None limiting examples of compositions and methods for making and using arrays are described in U.S. Pat. Nos. 5,202,231, 5,695,940, 5525464, 5445934, 5744305, 5677195, 5800992, 5871928, 5795716, 5700637, 6054270, 5807522, and 6110426, each of which is incorporated by reference herein in its entirety for all purposes.

The present invention is based at least in part, on the surprising discovery that specific biomarkers can be employed to evaluate, predict, and determine efficacy of treatment for a sialic acid deficiency treatment. Compared to previous technologies, which relied on muscle tissue biopsies, this discovery is less invasive and thus much easier to use for regularly monitoring responsiveness or efficacy of a sialic acid therapy (SAT) in a subject, determining whether a subject with a sialic acid deficiency is suitable for sialic acid deficiency treatment and using that information to improve treatment of such subjects, among other methods described herein. In some embodiments, the prediction or determination is made before the treatment. In some embodiments, the prediction or determination is based on an increase or decrease in the baseline level of one or more SAT biomarkers in the biological sample compared to a predetermined standard level. In some embodiments, the prediction/determination is made within a short period of time after the treatment starts. Within the short period of time, when there is no obvious alteration of symptoms of the diseases due to the treatment yet, it is hard to predict or determine the efficacy of the treatment and if the treatment is suitable for the subject by other methods. However, using the methods of the present invention, the prediction or determination can be quickly made based on the presence or absence of a normalization or stabilization in the level of one or more SAT biomarkers toward a predetermined standard level in the biological sample.

As used herein, the term “marker” or “biomarker” encompasses a broad range of intra- and extra-cellular events as well as whole-organism physiological changes. A marker may be represent essentially any aspect of cell function, for example, but not limited to, levels or rate of production of signaling molecules, transcription factors, metabolites, gene transcripts as well as post-translational modifications of proteins. Marker may include partial and/or whole genome analysis of transcript levels, rates, and/or stability, and partial and/or whole proteome analysis of protein levels, activity and/or modifications. A signature may refer to a gene or gene product which is up- or down-regulated in a subject to be treated compared to clinically normal subjects. A signature may also refer to a gene or gene product which is up- or down-regulated in a treated subject having the disease compared to an untreated subjects. That is, the gene or gene product is sufficiently specific to the treated cell that it may be used, optionally with other genes or gene products, to identify, predict, or detect efficacy of a small molecule. Thus, in some embodiments, a signature is a gene or gene product that is characteristic of efficacy of a compound in a diseased cell or the response of that diseased cell to treatment by the compound.

As used herein, sialic acid deficiency treatment refers to any treatment that can either increase the endogenous sialic acid level, and/or activating the sialic acid signaling transduction pathway.

As used herein, the term “sialic acid” refers to sialic acid, any functional derivatives thereof, analogs thereof, any anomers thereof, such as those disclosed in U.S. Pat. Nos. 4,694,076, 8,293,888, 6,288,041, 5,783,564, 7,875,708, 5,712,254, 5,438,125, 4,935,506, 5,077,397, RE34,091, 5,243,035, 4,918,177, 6,444,649, 4,968,786, 5,834,423, 5,621,086, 5,034,516, 7,413,729, 4,990,603, 4,914,035, 5,350,841, 8,217,154, 7,807,824, 5,519,007, 5,792,858, 5,459,031, 8,097,591, 5,453,272, 4,457,865, 5,233,033, 7,867,541, 7,951,410, 5,792,842, 4,520,111, 5,849,717, 5,679,321, 5,851,395, 5,750,508, 5,192,661, 5,658,880, 5,405,753, 4,963,653, 5,679,645, 8,323,654, 5,330,897, 5,334,514, 5,908,766, 7,803,583, 5,660,992, 8,148,335, and WO/2013/063149, each of which is incorporated by reference in its entirety. As used herein, the word “derivative” as used herein includes derivatives, analogs, prodrugs, and unnatural precursors.

In some embodiments, the invention provides methods for monitoring responsiveness or efficacy of a sialic acid deficiency treatment in a subject suffering from a sialic acid deficiency comprising detecting the level of one or more sialic acid therapy (SAT) biomarkers in a biological sample from the subject treated for a sialic acid deficiency, wherein an increase or decrease in the level of one or more SAT biomarkers indicates efficacy of treatment with the sialic acid deficiency treatment. The term “sample” or “biological sample” as used herein, refers to a sample obtained from an organism or from components (e.g., cells) of an organism. The sample may be of any biological tissue or fluid. The sample may be a sample which is derived from a patient. Such samples include, but are not limited to, sputum, blood, blood cells (e.g., white blood cells), tissue or biopsy samples (e.g., tumor biopsy), urine, peritoneal fluid, and pleural fluid, patient derived xenografts (PDXs), or cells therefrom. Biological samples may also include sections of tissues such as frozen sections taken for histological purposes.

In some embodiments, a collection of activity profiles of a panel of biomarkers is provided. As used herein, the term “activity profile” refers to a set of data representing distinctive features or characteristics of one or more gene markers. Such features or characteristics include, but are not limited to, transcript abundance, transcript stability, transcription rate, translation rate, post-translation modification, protein abundance, protein stability, and/or protein enzymatic activity, etc. In some embodiments, the activity profile comprises data related to gene expression level of each biomarker. In some embodiments, the collection comprises activity profiles is obtained from a specific population of subjects. In some embodiments, the specific population of subjects consists of clinically normal subjects.

In some embodiments, the collection comprises activity profiles that are statistically homogeneous in one or more aspects, e.g., statistically homogeneous in one or more quantitative or semi-quantitative parameters describing the features and characteristics of the activity profiles. In some embodiments, the quantitative parameters include, but are not limited to, transcript abundance, transcript stability, transcription rate, translation rate, post-translation modification, protein abundance, protein stability, and/or protein enzymatic activity, etc. Whether a group of activity profiles are statistically homogeneous or not in one or more aspects can be determined by any suitable statistic test and/or algorithm known to one skilled in the art.

In some embodiments, one or more of the biomarkers increase its activity in response to the treatment. In some embodiments, one or more of the biomarkers decrease its activity in response to the treatment. In some embodiments, one or more of the biomarkers remains its activity in response to the treatment. As used herein, the term “gene activity” refers to gene expression level, RNA activity level, or protein activity level. As used herein, the term “RNA activity level refers to mRNA abundance, synthesis rate, and/or stability, etc. As used herein, the term “protein activity level” refers to protein abundance, synthesis rate, stability, enzymatic activity, phosphorylation rate, etc.

In some embodiments, one or more of the biomarkers in a subject increases its activity and goes toward the level of a predetermined standard level. In some embodiments, one or more of the biomarkers in a subject decreases its activity and goes toward the level of a predetermined standard level. As used herein, when the level of a biomarker goes toward the level of a predetermined standard level, it is called normalization. In some embodiments, the normalization biomarkers of the present invention include, but art not limited to Chromogranin-A (CgA), Epithelial-Derived Neutrophil-Activating Protein 78 (ENA-78) Lactoylglutathione lyase (LGL), Latency-Associated Peptide of Transforming Growth Factor beta 1 (LAP TGF-b1), Myoglobin, Neuronal Cell Adhesion Molecule (Nr-CAM), and Plasminogen Activator Inhibitor 1 (PAI-1). In some embodiments, one or more of the biomarkers in a subject been treated with a drug has a less change in its activity compared to the same biomarker in a subject treated with a placebo which goes further away from a predetermined standard level. In some embodiments, the same biomarker in a subject treated with a drug reduces its speed of going away from the predetermined standard level compared to that of a placebo treatment. As used herein, when the level of a biomarker reduces its speed of going away from the level of a predetermined standard level, it is called stabilization. In some embodiments, the stabilization biomarkers of the present invention include, but art not limited to Macrophage Colony-Stimulating Factor 1 (M-CSF), Matrix Metalloproteinase-9, total (MMP-9, total), Receptor tyrosine-protein kinase erbB-3 (ErbB3), and Vascular Endothelial Growth Factor C (VEGF-C).

In some embodiments, the collection of activity profiles of one or more gene markers of the present invention is obtained from one or more tests. The test can be performed by the subject himself/herself, by a doctor, by a nurse, by a test lab, by a healthcare provider, or any other parties capable of doing the test. The test results containing the collection of activity profiles can be then analyzed by the same party or by a second party, such as the subject himself/herself, a doctor, a nurse, a test lab, a healthcare provider, a physician, a clinical trial personnel, a hospital, a lab, a research institute, or any other parties capable of analyzing the test to determine if the subject is responsive to the drug.

In some embodiments, the present invention provides methods for determining the treatment regimen for treating a sialic acid deficiency. The methods include detecting the level of one or more SAT biomarkers in a biological sample from a subject treated for a sialic acid deficiency and determining a treatment regimen based on an increase or decrease in the level of one or more SAT biomarkers in the biological sample. In some embodiments, the treatment regimen is continued when the level of one or more biomarkers of the present invention goes toward the level of a predetermined standard level, or reduces the speed of going away from the predetermined standard level compared to that of a placebo treatment.

In some other embodiments, the present invention provides methods for predicting the treatment efficacy of a sialic acid deficiency treatment. The method includes detecting the level of one or more SAT biomarkers in a biological sample from a subject, wherein an increase or decrease in the level compared to a predetermined standard level is predictive of the treatment efficacy of the sialic acid deficiency treatment. In some embodiments, the level of one or more SAT biomarkers in the biological sample from the subject is detected before the treatment, and the treatment is determined to be effective if the baseline level of one or more SAT biomarkers is increased or decreased compared to a predetermined standard level. In some embodiments, the level of one or more SAT biomarkers in the biological sample from the subject is detected a short period of time after the treatments. For example, the detecting step is conducted right after at least one, two, three, four, five, six, seven, eight, nine, ten, or more administrations of the sialic acid deficiency treatment. Each administration is spaced by a half day, one day, two days, three days, four days, five days, six days, one week, two weeks, three weeks, four weeks, or more. Within such short period of time, there is no obvious alteration of symptoms of the diseases due to the treatment yet, but the efficacy of the treatment be can be predicted based on the level of one or more biomarkers compared to the predetermined standard level. In some embodiments, the level of one or more biomarkers in a subject group treated with placebo is also included as a control. In some embodiments, the treatment is determined to be effective if one or more biomarkers of the present invention goes toward the level of a predetermined standard level, or reduces the speed of going away from the predetermined standard level compared to that of a placebo treatment.

In some embodiments, at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at last nineteen, at least twenty, at least twenty five, at least thirty, at least thirty five, at least forty, at least forty five, at least fifty, at least fifty five, at least sixty, at least sixty five, at least seventy, at least seventy five, at least eighty, at least eighty five, at least ninety, at least ninety five, at least one hundred or more biomarkers of the present invention provide a pattern that indicates the treatment is effective. The more biomarkers that give such a pattern, the more accurate the prediction is.

In some embodiments, the methods of the present invention comprise detecting the level of one or more biomarkers in a biological sample of a subject before the treatment. In some embodiments, the methods of the present invention comprise detecting the level of one or more biomarkers in a biological sample of a subject after the treatment. In some embodiments, the detecting step is conducted within a short period of time after the treatment starts. In some embodiments, within the short period of time, there is no obvious alteration of symptoms of the diseases due to the treatment yet. For example, the detecting step is conducted right after at least one administration of the sialic acid deficiency treatment. In some embodiments, the short period of time lasts for about 1 week, 2 weeks, 3 weeks, 4 weeks, 1 month, 2 months, 3 months, 4 months, 5 months, 6 months, 7 months, 8 months, 9 months, 10 months, 11 months, one year, two years, three years, or more.

In yet some other embodiments, the present invention provides methods for determining whether a subject with a sialic acid deficiency is suitable for a sialic acid deficiency treatment. The methods include detecting the level of one or more SAT biomarkers in a sample from the subject, wherein an increase or decrease in the level of one or more SAT biomarkers compared to a predetermined standard level indicates a subject is suitable for a sialic acid deficiency treatment. In some embodiments, the patient is determined to be suitable for the treatment when the level of one or more biomarkers of the present invention in the patient goes toward the level of a predetermined standard level, or reduces the speed of going away from the predetermined standard level compared to that of a placebo treatment.

In yet further embodiments the present invention provides methods for treating a subject with a sialic acid deficiency. The methods include detecting the level of one or more SAT biomarkers in a biological sample from the subject and administering a sialic acid deficiency treatment to the subject if the level of one or more SAT biomarkers is increased or decreased compared to a predetermined standard level. In some embodiments, the dosage of the treatment is determined by monitoring the level of one or more SAT biomarkers. In some embodiments, the dosage of the treatment is modified according to the level of one or more SAT biomarkers in the sample. In some embodiments, the dosage of the treatment can be raised when the subject is less sensitive to the treatment compared to a clinically normal group of subjects. In some embodiments, the dosage can be reduced when the subject is more sensitive to the treatment compared to a clinically normal group of subjects.

Embodiments of the present invention provide methods for treating a subject with a sialic acid deficiency. These methods comprise receiving information on the level of one or more SAT biomarkers in a biological sample from the subject, and administering a sialic acid deficiency treatment to the subject if the level of one or more SAT biomarkers is increased or decreased compared to a predetermined standard level. In some embodiments the information is in the form of data regarding the level of the SAT biomarker. In some embodiments, the information is in the form of data comparing the level of the SAT biomarker to a predetermined standard level.

In still other embodiments, the present invention provides methods for providing data. These methods include detecting the level of one or more SAT biomarkers in a sample from a subject and providing the information regarding the level of one or more SAT biomarkers to a healthcare provider for diagnosis or treatment of the subject. In some embodiments, the biological sample is received from a healthcare provider.

In yet other embodiments, the present invention provides methods of providing useful information for predicting or determining the treatment efficacy of a sialic acid deficiency treatment comprising determining the level of one or more SAT biomarkers from a biological sample of a subject and providing the level of one or more SAT biomarkers to an entity that provides a prediction or determination of the treatment efficacy based on an increase or decrease in the level of one or more of the SAT biomarkers in a subject. In some embodiments the subject is treated with the sialic acid deficiency treatment. In some embodiments the level of one or more SAT biomarkers is determined before treatment with the sialic acid deficiency treatment. In some embodiments the level of one or more SAT biomarkers is determined after treatment with the sialic acid deficiency treatment. In some embodiments the level of one or more SAT biomarkers is compared between before treatment with the sialic acid deficiency treatment and after treatment with the sialic acid deficiency treatment. In some embodiments the level of one or more SAT biomarkers is determined before and/or after treatment with the sialic acid deficiency treatment and is compared to a predetermined standard level.

In still other embodiments, the present invention provides a combination of tests useful for predicting or determining the treatment efficacy of a sialic acid deficiency treatment comprising a first test for detecting the level of one SAT biomarker from a biological sample from a subject and a second test for detecting the level of a second SAT biomarker of said biological sample, wherein the first SAT biomarker is different from the second SAT biomarker.

Sialic acid deficiencies of the present invention refers to symptoms associated with any abnormal activity of the sialic acid when compared to a normal subject, which include but are not limited to Hereditary Inclusion Body Myopathy (HIBM), Nonaka myopathy, or Distal Myopathy with Rimmed Vacuoles (DMRV). In some embodiments, the sialic acid deficiency is Hereditary Inclusion Body Myopathy (HIBM). In some embodiments, the sialic acid deficiency is due to a mutation and/or deficiency in the GNE gene, which encodes the bi-functional enzyme UDP-GlcNAc 2-epimerase/ManNAc kinase. In some cases, the sialic acid deficiency is due to a homogeneous or homozygous mutation and/or deficiency in the GNE gene. In some embodiments, the sialic acid deficiency is due to a heterogeneous or heterozygous mutation and/or deficiency in the GNE. In some embodiments, the sialic acid deficiency is not due to a mutation and/or deficiency in the GNE gene.

SAT biomarkers contemplated for use with the methods of the present invention can include but are not limited to those listed in Tables 2-14.

In some embodiments, SAT biomarkers can include but are not limited to 6Ckine, Agouti-Related Protein (AgRP), Aldose Reductase, Alpha-1-Antichymotrypsin (AACT), Alpha-2-Macroglobulin (A2Macro), Amphiregulin (AR), Angiogenin, Angiotensin-Converting Enzyme (ACE), Angiotensinogen, Apolipoprotein A-I (Apo A-I), Apolipoprotein A-II (Apo A-II), Apolipoprotein B (Apo B), Apolipoprotein C-I (Apo C-I), Apolipoprotein C-III (Apo C-III), Apolipoprotein E (Apo E), AXL Receptor Tyrosine Kinase (AXL), B cell-activating factor (BAIT), Brain-Derived Neurotrophic Factor (BDNF), Calbindin, Carcinoembryonic Antigen (CEA), CD40 Ligand (CD40-L), CD5 Antigen-like (CD5L), Cellular Fibronectin (cFib), Chromogranin-A (CgA), Collagen IV, Complement C3 (C3), Cortisol (Cortisol), Creatine Kinase-MB (CK-MB), E-Selectin, EN-RAGE, Endoglin, Endostatin, Eotaxin-1, Epidermal Growth Factor (EGF), Epithelial-Derived Neutrophil-Activating Protein 78 (ENA-78), Erythropoietin (EPO), Factor VII, Fatty Acid-Binding Protein, adipocyte (FABP, adipocyte), Fatty Acid-Binding Protein, heart (FABP, heart), Fatty Acid-Binding Protein, liver (FABP, liver), Fibrinogen, Fibroblast Growth Factor 4 (FGF-4), Galectin-3, Glucagon-like Peptide 1, total (GLP-1 total), Glutathione S-Transferase alpha (GST-alpha), Granulocyte Colony-Stimulating Factor (G-CSF), Granulocyte-Macrophage Colony-Stimulating Factor (GM-CSF), Haptoglobin, HE4, Heparin-Binding EGF-Like Growth Factor (HB-EGF), Hepatocyte Growth Factor (HGF), Hepatocyte Growth Factor receptor (HGF receptor), Human Epidermal Growth Factor Receptor 2 (HER-2), Immunoglobulin A (IgA), Insulin-like Growth Factor-Binding Protein 2 (IGFBP-2), Insulin-like Growth Factor-Binding Protein 3 (IGFBP-3), Insulin-like Growth Factor Binding Protein 4 (IGFBP-4), Insulin-like Growth Factor Binding Protein 6 (IGFBP-6), Intercellular Adhesion Molecule 1 (ICAM-1), Interferon-inducible T-cell alpha chemoattractant (ITAC), Interleukin-2 receptor alpha (IL-2 receptor alpha), Interleukin-3 (IL-3), Interleukin-4 (IL-4), Interleukin-5 (IL-5), Interleukin-6 receptor subunit beta (IL-6R beta), Interleukin-7 (IL-7), Interleukin-8 (IL-8), Interleukin-15 (IL-15), Kallikrein 5, Lactoylglutathione lyase (LGL), Latency-Associated Peptide of Transforming Growth Factor beta 1 (LAP TGF-b1), Lectin-Like Oxidized LDL Receptor 1 (LOX-1), Leptin, Macrophage Colony-Stimulating Factor 1 (M-CSF), Macrophage Inflammatory Protein-1 alpha (MIP-1 alpha), Macrophage Inflammatory Protein-3 alpha (MIP-3 alpha), Macrophage-Stimulating Protein (MSP), Malondialdehyde-Modified Low-Density Lipoprotein (MDA-LDL), Matrix Metalloproteinase-1 (MMP-1), Matrix Metalloproteinase-3 (MMP-3), Matrix Metalloproteinase-9, total (MMP-9, total), Mesothelin (MSLN), MHC class I chain-related protein A (MICA), Monocyte Chemotactic Protein 1 (MCP-1), Monocyte Chemotactic Protein 2 (MCP-2), Monocyte Chemotactic Protein 4 (MCP-4), Monokine Induced by Gamma Interferon (MIG), Myeloid Progenitor Inhibitory Factor 1 (MPIF-1), Myeloperoxidase (MPO), Myoglobin, Neuron-Specific Enolase (NSE), Neuronal Cell Adhesion Molecule (Nr-CAM), Neutrophil Gelatinase-Associated Lipocalin (NGAL), Pepsinogen I (PGI), Peptide YY (PYY), Plasminogen Activator Inhibitor 1 (PAI-1), Platelet-Derived Growth Factor BB (PDGF-BB), Pregnancy-Associated Plasma Protein A (PAPP-A), Progesterone, Prostatic Acid Phosphatase (PAP), Protein S100-A4 (S100-A4), Protein S 100-A6 (S100-A6), Receptor for advanced glycosylation end products (RAGE), Receptor tyrosine-protein kinase erbB-3 (ErbB3), Resistin, Serum Glutamic Oxaloacetic Transaminase (SGOT), Sortilin, Stem Cell Factor (SCF), Stromal cell-derived factor-1 (SDF-1) Superoxide Dismutase 1, soluble (SOD-1), T-Cell-Specific Protein RANTES (RANTES), T Lymphocyte-Secreted Protein 1-309 (1-309), Tenascin-C (TN-C), Thrombomodulin (TM), Thrombopoietin (TPO), Thrombospondin-1, Thyroglobulin (TG), Thyroxine-Binding Globulin (TBG), Tissue Factor (TF), Tissue Inhibitor of Metalloproteinases 1 (TIMP-1), Tumor Necrosis Factor alpha (TNF-alpha), Tumor Necrosis Factor Receptor I (TNFR1), Urokinase-type Plasminogen Activator (uPA), Urokinase-type Plasminogen Activator Receptor (uPAR), Vascular Endothelial Growth Factor (VEGF), Vascular Endothelial Growth Factor B (VEGF-B), Vascular Endothelial Growth Factor C (VEGF-C), Vascular Endothelial Growth Factor D (VEGF-D), Vascular Endothelial Growth Factor Receptor 1 (VEGFR-1), Vascular Endothelial Growth Factor Receptor 2 (VEGFR-2), Vascular Endothelial Growth Factor Receptor 3 (VEGFR-3), Vitamin K-Dependent Protein S (VKDPS), Vitronectin and von Willebrand Factor (vWF). See for example, Table 2 biomarkers.

In some embodiments, SAT biomarkers can include but are not limited to Agouti-Related Protein (AgRP), Amphiregulin (AR), Brain-Derived Neurotrophic Factor (BDNF), CD40 Ligand (CD40-L), Chromogranin-A (CgA), Cortisol (Cortisol), Creatine Kinase-MB (CK-MB), Epidermal Growth Factor (EGF), Epithelial-Derived Neutrophil-Activating Protein 78 (ENA-78), Fibroblast Growth Factor 4 (FGF-4), Insulin-like Growth Factor Binding Protein 6 (IGFBP-6), Interleukin-3 (IL-3), Interleukin-5 (IL-5), Interleukin-7 (IL-7), Interleukin-8 (IL-8), Kallikrein 5, Latency-Associated Peptide of Transforming Growth Factor beta 1 (LAP TGF-b1), Macrophage Colony-Stimulating Factor 1 (M-CSF), Macrophage Inflammatory Protein-3 alpha (MIP-3 alpha), Matrix Metalloproteinase-1 (MMP-1), Matrix Metalloproteinase-3 (MMP-3), Matrix Metalloproteinase-9, total (MMP-9, total), Myeloperoxidase (MPO), Myoglobin, N-terminal prohormone of brain natriuretic peptide (NT proBNP), Neuron-Specific Enolase (NSE), Neuronal Cell Adhesion Molecule (Nr-CAM), Plasminogen Activator Inhibitor 1 (PAI-1), Platelet-Derived Growth Factor BB (PDGF-BB), Protein S100-A4 (S100-A4), Protein S100-A6 (S100-A6), Receptor tyrosine-protein kinase erbB-3 (ErbB3), Serum Glutamic Oxaloacetic Transaminase (SGOT), T-Cell-Specific Protein RANTES (RANTES), Thrombospondin-1, Thyroglobulin (TG) and Vascular Endothelial Growth Factor C (VEGF-C). See for example, Table 7.

In some embodiments, SAT biomarkers can include but are not limited to Brain-Derived Neurotrophic Factor (BDNF), CD40 Ligand (CD40-L), Chromogranin-A (CgA), Creatine Kinase-MB (CK-MB), Insulin-like Growth Factor Binding Protein 6 (IGFBP-6), Interleukin-3 (IL-3), Interleukin-5 (IL-5), Latency-Associated Peptide of Transforming Growth Factor beta 1 (LAP TGF-b1), Macrophage Colony-Stimulating Factor 1 (M-CSF), Matrix Metalloproteinase-9, total (MMP-9, total), Myoglobin, Neuron Specific Enolase (NSE), Plasminogen Activator Inhibitor 1 (PAI-1), Platelet-Derived Growth Factor BB (PDGF-BB), Receptor tyrosine-protein kinase erbB-3 (ErbB3), T-Cell-Specific Protein RANTES (RANTES), Thrombospondin-1 and Vascular Endothelial Growth Factor C (VEGF-C). See for example, Table 8.

In some embodiments, SAT biomarkers can include but are not limited to Brain-Derived Neurotrophic Factor (BDNF), CD40 Ligand (CD40-L), Chromogranin-A (CgA), Latency-Associated Peptide of Transforming Growth Factor beta 1 (LAP TGF-b1), Matrix Metalloproteinase-9, total (MMP-9, total), Myoglobin, Plasminogen Activator Inhibitor 1 (PAI-1), Platelet-Derived Growth Factor BB (PDGF-BB), Receptor tyrosine-protein kinase erbB-3 (ErbB3), T-Cell-Specific Protein RANTES (RANTES) and Thrombospondin-1. See for example, Table 9.

In some embodiments, SAT biomarkers can include but are not limited to 6Ckine, Adiponectin Agouti-Related Protein (AGRP), Aldose Reductase, Alpha-1-Antichymotrypsin (AACT), Alpha-1-Antitrypsin (AAT), Alpha-1-Microglobulin (A1Micro), Alpha-2-Macroglobulin (A2Macro), Alpha-Fetoprotein (AFP), Amphiregulin (AR), Angiogenin, Angiopoietin-2 (ANG-2), Angiotensin-Converting Enzyme (ACE), Angiotensinogen, Annexin A1 (ANXA1), Apolipoprotein A-I (Apo A-I), Apolipoprotein A-II (Apo A-II), Apolipoprotein A-IV (Apo A-IV), Apolipoprotein B (Apo B), Apolipoprotein C-I (Apo C-I), Apolipoprotein C-III (Apo C-III), Apolipoprotein D (Apo D), Apolipoprotein E (Apo E), Apolipoprotein H (Apo H), Apolipoprotein(a) (Lp(a)), AXL Receptor Tyrosine Kinase (AXL), B cell-activating factor (BAFF), B Lymphocyte Chemoattractant (BLC), Bcl-2-like protein 2 (Bcl2-L-2), Beta-2-Microglobulin (B2M), Betacellulin (BTC), Bone Morphogenetic Protein 6 (BMP-6), Brain-Derived Neurotrophic Factor (BDNF), Calbindin, Calcitonin, Cancer Antigen 125 (CA-125), Cancer Antigen 15-3 (CA-15-3), Cancer Antigen 19-9 (CA-19-9), Cancer Antigen 72-4(CA-72-4), Carcinoembryonic Antigen (CEA), Cathepsin D, CD 40 antigen (CD40), CD40 Ligand (CD40-L), CD5 (CD5L), Cellular Fibronectin (cFib), Chemokine CC-4 (HCC-4), Chromogranin-A (CgA), Ciliary Neurotrophic Factor (CNTF), Clusterin (CLU), Collagen IV, Complement C3 (C3), Complement Factor I-I, Connective Tissue Growth Factor (CTGF), Cortisol (Cortisol), C-peptide, C-Reactive Protein (CRP), Creatine Kinase-MB (CK-MB), Cystatin-C, Endoglin, Endostatin, Endothelin-1 (ET-1), EN-RAGE Eotaxin-1, Eotaxin-2, Eotaxin-3, Epidermal Growth Factor (EGF), Epidermal Growth Factor Receptor (EGFR), Epiregulin (EPR), Epithelial cell adhesion molecule (EpCam), Epithelial-Derived Neutrophil-Activating Protein 78 (ENA-78), Erythropoietin (EPO), E-Selectin, Ezrin, Factor VII, Fas Ligand (FasL), FASLG Receptor (FAS), Fatty Acid-Binding Protein, adipocyte (FABP, adipocyte), Fatty Acid-Binding Protein, heart (FABP, heart), Fatty Acid-Binding Protein, liver (FABP, liver), Ferritin (FRTN), Fetuin-A, Fibrinogen, Fibroblast Growth Factor 4 (FGF-4), Fibroblast Growth Factor basic (FGF-basic), Fibulin-1C (Fib-1C), Follicle-Stimulating Hormone (FSH), Galectin-3, Gelsolin, Glucagon, Glucagon-like Peptide 1, total (GLP-1 total), Glucose-6-phosphate Isomerase (G6PI), Glutamate-Cysteine Ligase Regulatory subunit (GCLR), Glutathione S-Transferase alpha (GST-alpha), Glutathione S-Transferase Mu 1 (GST-M1), Granulocyte Colony-Stimulating Factor (G-CSF), Granulocyte-Macrophage Colony-Stimulating Factor (GM-CSF), Growth Hormone (GH), Haptoglobin, HE4, Heat Shock Protein 60 (HSP-60), Heparin-Binding EGF-Like Growth Factor (HB-EGF), Hepatocyte Growth Factor (HGF), Hepatocyte Growth Factor receptor (HGF receptor), Hepsin, Human Chorionic Gonadotropin beta (hCG), Human Epidermal Growth Factor Receptor 2 (HER-2), Immunoglobulin A (IgA), Immunoglobulin E (IgE), Immunoglobulin M (IGM), Insulin, Insulin-like Growth Factor Binding Protein 4 (IGFBP4), Insulin-like Growth Factor Binding Protein 5 (IGFBP5), Insulin-like Growth Factor Binding Protein 6 (IGFBP6), Insulin-like Growth Factor-Binding Protein 1 (IGFBP-1), Insulin-like Growth Factor-Binding Protein 2 (IGFBP-2), Insulin-like Growth Factor-Binding Protein 3 (IGFBP-3), Intercellular Adhesion Molecule 1 (ICAM-1), Interferon gamma (IFN-gamma), Interferon gamma Induced Protein 10 (IP-10), Interferon-inducible T-cell alpha chemoattractant (ITAC) Interleukin-1 alpha (IL-1 alpha), Interleukin-1 beta (IL-1 beta), Interleukin-1 receptor antagonist (IL-1ra), Interleukin-10 (IL-10), Interleukin-12 Subunit p40 (IL-12p40), Interleukin-12 Subunit p70 (IL-12p70), Interleukin-13 (IL-13), Interleukin-15 (IL-15), Interleukin-16 (IL-16), Interleukin-18 (IL-18), Interleukin-2 (IL-2), Interleukin-2 receptor alpha (IL-2 receptor alpha), Interleukin-25 (IL-25), Interleukin-3 (IL-3), Interleukin-4 (IL-4), Interleukin-5 (IL-5), Interleukin-6 (IL-6), Interleukin-6 receptor (IL-6r), Interleukin-6 receptor subunit beta(IL-6R beta), Interleukin-7 (IL-7), Interleukin-8 (IL-8), Kallikrein 5, Kallikrein-7 (KLK-7), Kidney Injury Molecule-1 (KIM-1), Lactoylglutathione lyase (LGL), Latency-Associated Peptide of Transforming Growth Factor beta 1 (LAP TGF-b1), Lectin-Like Oxidized LDL Receptor 1 (LOX-1), Leptin, Luteinizing Hormone (LH), Lymphotactin, Macrophage Colony-Stimulating Factor 1 (M-CSF), Macrophage inflammatory protein 3 beta (MIP-3 beta), Macrophage Inflammatory Protein-1 alpha (MIP-1 alpha), Macrophage Inflammatory Protein-1 beta (MIP-1 beta), Macrophage Inflammatory Protein-3 alpha (MIP-3 alpha), Macrophage Migration Inhibitory Factor (MIF), Macrophage-Derived Chemokine (MDC), Macrophage-Stimulating Protein (MSP), Malondialdehyde-Modified Low-Density Lipoprotein (MDA-LDL), Maspin, Matrix Metalloproteinase-1 (MMP-1), Matrix Metalloproteinase-10 (MMP-10), Matrix Metalloproteinase-2 (MMP-2), Matrix Metalloproteinase-3 (MMP-3), Matrix Metalloproteinase-7 (MMP-7), Matrix Metalloproteinase-9 (MMP-9), Matrix Metalloproteinase-9, total (MMP-9, total), Mesothelin (MSLN), MHC class I chain-related protein 1 (MICA), Monocyte Chemotactic Protein 1 (MCP-1), Monocyte Chemotactic Protein 2 (MCP-2), Monocyte Chemotactic Protein 3 (MCP-3), Monocyte Chemotactic Protein 4 (MCP-4), Monokine Induced by Gamma Interferon (MIG), Myeloid Progenitor Inhibitory Factor 1 (MPIF-1), Myeloperoxidase (MPO), Myoglobin, Nerve Growth Factor beta (NGF-beta), Neuron Specific Enolase (NSE), Neuronal Cell Adhesion Molecule (Nr-CAM), Neuropilin-1Neutrophil Gelatinase-Associated Lipocalin (NGAL), N-terminal prohormone of brain natriuretic peptide (NT proBNP), Nucleoside diphosphate kinase B (NDK B), Osteopontin, Osteoprotegerin (OPG), Pancreatic Polypeptide (PPP), Pepsinogen I (PGI), Peptide YY (PYY), Peroxiredoxin 4 (Prx-IV), Phosphoserine Aminotransferase (PSAT), Placenta Growth Factor (PLGF), Plasminogen Activator Inhibitor 1 (PAI-1), Platelet-Derived Growth Factor BB (PDGF-BB), Pregnancy-Associated Plasma Protein A (PAPP-A), Progesterone, Proinsulin (Intact), Proinsulin (Total), Prolactin (PRL), Prostasin, Prostate-Specific Antigen Free (PSA-f), Prostatic Acid Phosphatase (PAP), Protein S100-A4 (S100-A4), Protein S100-A6 (S100-A6), Pulmonary and Activation-Regulated Chemokine (PARC), Receptor for advanced glycosylation end products (RAGE), Receptor tyrosine-protein kinase erbB-3 (ErbB3), Resistin, S100 calcium-binding protein B (S100-B), Secretin, Serotransferrin (Transferrin), Serum Amyloid P-Component (SAP), Serum Glutamic Oxaloacetic Transaminase (SGOT), Sex Hormone-Binding Globulin (SHBG), Sortilin, Squamous Cell Carcinoma Antigen-1 (SCCA-1), Stem Cell Factor (SCF), Stromal cell-derived factor-1 (SDF-1), Superoxide Dismutase 1, Soluble (SOD-1), T Lymphocyte-Secreted Protein 1-309 (1-309), Tamm-Horsfall Urinary Glycoprotein (THP), T-Cell-Specific Protein RANTES (RANTES), Tenascin-C (TN-C), Testosterone (Total), Tetranectin, Thrombomodulin (TM), Thrombopoietin, Thrombospondin-1, Thyroglobulin (TG), Thyroid-Stimulating Hormone (TSH), Thyroxine-Binding Globulin (TBG), Tissue Factor (TF), Tissue Inhibitor of Metalloproteinases 1 (TIMP-1), Tissue type Plasminogen activator (tPA), TNF-Related Apoptosis-Inducing Ligand Receptor 3 (TRAIL-R3), Transforming Growth Factor alpha (TGF-alpha), Transforming Growth Factor beta-3 (TGF-beta-3), Transthyretin (TTR), Trefoil Factor 3 (TFF3), Tumor Necrosis Factor alpha (TNF-alpha), Tumor Necrosis Factor beta (TNF-beta), Tumor Necrosis Factor Receptor 2 (TNFR2), Tumor Necrosis Factor Receptor I (TNF R1), Tyrosine kinase with Ig and EGF homology domains 2 (TIE-2), Urokinase-type Plasminogen Activator (uPA), Urokinase-type Plasminogen Activator Receptor(uPAR), Vascular Cell Adhesion Molecule-1 (VCAM-1), Vascular Endothelial Growth Factor (VEGF), Vascular Endothelial Growth Factor B (VEGF-B), Vascular Endothelial Growth Factor C (VEGF-C), Vascular Endothelial Growth Factor D (VEGF-D), Vascular Endothelial Growth Factor Receptor 1 (VEGFR-1), Vascular Endothelial Growth Factor Receptor 2 (VEGFR-2), Vascular Endothelial Growth Factor Receptor 3 (VEGFR-3), Vitamin K-Dependent Protein S (VKDPS), Vitronectin, von Willebrand Factor (vWF) and YKL-40.

In some embodiments, SAT biomarkers can include but are not limited to 6Ckine, Adiponectin, Agouti-Related Protein (AgRP), Aldose Reductase, Alpha-1-Antichymotrypsin (AACT), Alpha-1-Antitrypsin (AAT), Alpha-1-Microglobulin (A1Micro), Alpha-2-Macroglobulin (A2Macro), Alpha-Fetoprotein (AFP), Amphiregulin (AR), Angiogenin, Angiopoietin-2 (ANG-2), Angiotensin-Converting Enzyme (ACE), Angiotensinogen, Apolipoprotein(a) (Lp(a)), Apolipoprotein A-I (Apo A-I), Apolipoprotein A-II (Apo A-II), Apolipoprotein A-IV (Apo A-IV), Apolipoprotein B (Apo B), Apolipoprotein C-I (Apo C-I), Apolipoprotein C-III (Apo C-III), Apolipoprotein D (Apo D), Apolipoprotein E (Apo E), Apolipoprotein H (Apo H), AXL Receptor Tyrosine Kinase (AXL), B cell-activating factor (BAFF), B Lymphocyte Chemoattractant (BLC), Beta-2-Microglobulin (B2M), Betacellulin (BTC), Brain-Derived Neurotrophic Factor (BDNF), C-Peptide, C-Reactive Protein (CRP), Calbindin, Cancer Antigen 125 (CA-125), Cancer Antigen 15-3 (CA-15-3), Cancer Antigen 19-9 (CA-19-9), Cancer Antigen 72-4 (CA 72-4), Carcinoembryonic Antigen (CEA), Cathepsin D, CD5 Antigen-like (CD5L), CD 40 antigen (CD40), CD40 Ligand (CD40-L), Cellular Fibronectin (cFib), Chemokine CC-4 (HCC-4), Chromogranin-A (CgA), Ciliary Neurotrophic Factor (CNTF), Clusterin (CLU), Collagen IV, Complement C3 (C3), Complement Factor H—Related Protein 1 (CFHR1), Cortisol (Cortisol), Creatine Kinase-MB (CK-MB), Cystatin-C, E-Selectin, EN-RAGE, Endoglin, Endostatin, Eotaxin-1, Eotaxin-2, Eotaxin-3, Epidermal Growth Factor (EGF), Epidermal Growth Factor Receptor (EGFR), Epiregulin (EPR), Epithelial cell adhesion molecule (EpCam), Epithelial-Derived Neutrophil-Activating Protein 78 (ENA-78), Ezrin, Factor VII, Fas Ligand (FasL), FASLG Receptor (FAS), Fatty Acid-Binding Protein—adipocyte (FABP, adipocyte), Fatty Acid-Binding Protein—heart (FABP, heart), Fatty Acid-Binding Protein—liver (FABP, liver), Ferritin (FRTN), Fetuin-A, Fibrinogen, Fibroblast Growth Factor 4 (FGF-4), Fibroblast Growth Factor basic (FGF-basic), Fibulin-1C (Fib-1C), Follicle-Stimulating Hormone (FSH), Galectin-3, Gelsolin, Glucagon, Glucagon-like Peptide 1-active (GLP-1 active), Glucagon-like Peptide 1—total (GLP-1 total), Glucose-6-phosphate Isomerase (G6PI), Glutathione S-Transferase alpha (GST-alpha), Glutathione S-Transferase Mu 1 (GST-M1), Granulocyte Colony-Stimulating Factor (G-CSF), Granulocyte-Macrophage Colony-Stimulating Factor (GM-CSF), Growth Hormone (GH), Growth-Regulated alpha protein (GRO-alpha), Haptoglobin HE4, Heat Shock Protein 60 (HSP-60), Heparin-Binding EGF-Like Growth Factor (HB-EGF), Hepatocyte Growth Factor (HGF), Hepatocyte Growth Factor receptor (HGF receptor), Hepsin, Human Chorionic Gonadotropin beta (hCG), Human Epidermal Growth Factor Receptor 2 (HER-2), Immunoglobulin A (IgA), Immunoglobulin E (IgE), Immunoglobulin M (IgM), Insulin, Insulin-like Growth Factor-Binding Protein 1 (IGFBP-1), Insulin-like Growth Factor-Binding Protein 2 (IGFBP-2), Insulin-like Growth Factor-Binding Protein 3 (IGFBP-3), Insulin-like Growth Factor Binding Protein 4 (IGFBP4), Insulin-like Growth Factor Binding Protein 5 (IGFBP5), Insulin-like Growth Factor Binding Protein 6 (IGFBP6), Intercellular Adhesion Molecule 1 (ICAM-1), Interferon gamma (IFN-gamma), Interferon gamma Induced Protein 10 (IP-10), Interferon-inducible T-cell alpha chemoattractant (ITAC), Interleukin-1 alpha (IL-1 alpha, Interleukin-1 beta (IL-1 beta), Interleukin-1 receptor antagonist (IL-1ra), Interleukin-2 (IL-2), Interleukin-2 receptor alpha (IL-2 receptor alpha), Interleukin-3 (IL-3), Interleukin-4 (IL-4), Interleukin-5 (IL-5), Interleukin-6 (IL-6), Interleukin-6 receptor (IL-6r), Interleukin-6 receptor subunit beta (IL-6R beta), Interleukin-7 (IL-7), Interleukin-8 (IL-8), Interleukin-10 (IL-10), Interleukin-12 Subunit p40 (IL-12p40), Interleukin-12 Subunit p70 (IL-12p70), Interleukin-13 (IL-13), Interleukin-15 (IL-15), Interleukin-16 (IL-16), Interleukin-17 (IL-17), Interleukin-18 (IL-18), Interleukin-23 (IL-23), Kallikrein 5, Kallikrein-7 (KLK-7), Kidney Injury Molecule-1 (KIM-1), Lactoylglutathione lyase (LGL), Latency-Associated Peptide of Transforming Growth Factor beta 1 (LAP TGF-b1), Lectin-Like Oxidized LDL Receptor 1 (LOX-1), Leptin, Luteinizing Hormone (LH), Macrophage Colony-Stimulating Factor 1 (M-CSF), Macrophage-Derived Chemokine (MDC), Macrophage Inflammatory Protein-1 alpha (MIP-1 alpha), Macrophage Inflammatory Protein-1 beta (MIP-1 beta), Macrophage Inflammatory Protein-3 alpha (MIP-3 alpha), Macrophage inflammatory protein 3 beta (MIP-3 beta), Macrophage Migration Inhibitory Factor (MIF), Macrophage-Stimulating Protein (MSP), Malondialdehyde-Modified Low-Density Lipoprotein (MDA-LDL), Maspin, Matrix Metalloproteinase-1 (MMP-1), Matrix Metalloproteinase-3 (MMP-3), Matrix Metalloproteinase-7 (MMP-7), Matrix Metalloproteinase-9 (MMP-9), Matrix Metalloproteinase-9—total (MMP-9, total), Matrix Metalloproteinase-10 (MMP-10), Mesothelin (MSLN), MHC class I chain-related protein A (MICA), Monocyte Chemotactic Protein 1 (MCP-1), Monocyte Chemotactic Protein 2 (MCP-2), Monocyte Chemotactic Protein 3 (MCP-3), Monocyte Chemotactic Protein 4 (MCP-4), Monokine Induced by Gamma Interferon (MIG), Myeloid Progenitor Inhibitory Factor 1 (MPIF-1), Myeloperoxidase (MPO), Myoglobin, N-terminal prohormone of brain natriuretic peptide (NT proBNP), Nerve Growth Factor beta (NGF-beta), Neuron-Specific Enolase (NSE), Neuronal Cell Adhesion Molecule (Nr-CAM), Neuropilin-1, Neutrophil Gelatinase-Associated Lipocalin (NGAL), Osteopontin, Osteoprotegerin (OPG), Pancreatic Polypeptide (PPP), Pepsinogen I (PGI), Peptide YY (PYY), Phosphoserine Aminotransferase (PSAT), Placenta Growth Factor (PLGF), Plasminogen Activator Inhibitor 1 (PAI-1), Platelet-Derived Growth Factor BB (PDGF-BB), Progesterone, Proinsulin (Intact), Proinsulin (Total), Prolactin (PRL), Prostasin, Prostate-Specific Antigen, Free (PSA-f), Protein S100-A4 (S100-A4), Pulmonary and Activation-Regulated Chemokine (PARC), Receptor for advanced glycosylation end products (RAGE), Receptor tyrosine-protein kinase erbB-3 (ErbB3), Resistin, S 100 calcium-binding protein B (S100-B), Serotransferrin (Transferrin), Serum Amyloid P-Component (SAP), Sex Hormone-Binding Globulin (SHBG), Sortilin, Squamous Cell Carcinoma Antigen-1 (SCCA-1), Stem Cell Factor (SCF), Stromal cell-derived factor-1 (SDF-1), Superoxide Dismutase 1, soluble (SOD-1), T-Cell-Specific Protein RANTES (RANTES), T Lymphocyte-Secreted Protein 1-309 (1-309), Tamm-Horsfall Urinary Glycoprotein (THP), Tenascin-C (TN-C), Testosterone (Total), Tetranectin, Thrombomodulin (TM), Thrombospondin-1, Thyroglobulin (TG), Thyroid-Stimulating Hormone (TSH), Thyroxine-Binding Globulin (TBG), Tissue Inhibitor of Metalloproteinases 1 (TIMP-1), Tissue type Plasminogen activator (tPA), TNF-Related Apoptosis-Inducing Ligand Receptor 3 (TRAIL-R3), Transforming Growth Factor alpha (TGF-alpha), Transforming Growth Factor beta-3 (TGF-beta-3), Transthyretin (TTR), Trefoil Factor 3 (TFF3), Tumor Necrosis Factor alpha (TNF-alpha), Tumor Necrosis Factor beta (TNF-beta), Tumor Necrosis Factor Receptor I (TNF R1), Tumor necrosis factor receptor 2 (TNFR2), Tyrosine kinase with Ig and EGF homology domains 2 (TIE-2), Urokinase-type Plasminogen Activator (uPA), Urokinase-type plasminogen activator receptor (uPAR), Vascular Cell Adhesion Molecule-1 (VCAM-1), Vascular Endothelial Growth Factor (VEGF), Vascular endothelial growth factor B (VEGF-B), Vascular Endothelial Growth Factor C (VEGF-C), Vascular endothelial growth factor D (VEGF-D), Vascular Endothelial Growth Factor Receptor 1 (VEGFR-1), Vascular Endothelial Growth Factor Receptor 2 (VEGFR-2), Vascular endothelial growth factor receptor 3 (VEGFR-3), Vitamin D-Binding Protein (VDBP), Vitamin K-Dependent Protein S (VKDPS), Vitronectin, von Willebrand Factor (vWF) and YKL-40.

In some embodiments, SAT biomarkers can include but are not limited to 6Ckine, Aldose Reductase, Alpha-1-Microglobulin (A1Micro), Alpha-2-Macroglobulin (A2Macro), Angiogenin, Angiopoietin-2 (ANG-2), Angiotensin-Converting Enzyme (ACE), Apolipoprotein(a) (Lp(a)), Apolipoprotein A-I (Apo A-I), Apolipoprotein A-II (Apo A-II), Apolipoprotein A-IV (Apo A-IV), Apolipoprotein B (Apo B), Apolipoprotein C-III (Apo C-III), Apolipoprotein D (Apo D), Apolipoprotein H (Apo H), AXL Receptor Tyrosine Kinase (AXL), B cell-activating factor (BAFF), B Lymphocyte Chemoattractant (BLC), Beta-2-Microglobulin (B2M), Brain-Derived Neurotrophic Factor (BDNF), C-Peptide, Carcinoembryonic Antigen (CEA), Cathepsin D, CD5 Antigen-like (CD5L), CD 40 antigen (CD40), CD40 Ligand (CD40-L), Cellular Fibronectin (cFib), Chromogranin-A (CgA), Clusterin (CLU), Complement C3 (C3), Complement Factor H—Related Protein 1 (CFHR1), Cortisol (Cortisol), Creatine Kinase-MB (CK-MB), Cystatin-C, E-Selectin, EN-RAGE, Endoglin, Endostatin, Eotaxin-1, Epidermal Growth Factor (EGF), Epidermal Growth Factor Receptor (EGFR), Epithelial-Derived Neutrophil-Activating Protein 78 (ENA-78), Factor VII, FASLG Receptor (FAS), Fatty Acid-Binding Protein—adipocyte (FABP, adipocyte), Fetuin-A, Fibrinogen, Galectin-3, Gelsolin, Glucagon-like Peptide 1—total (GLP-1 total), Glutathione S-Transferase Mu 1 (GST-M1), Granulocyte Colony-Stimulating Factor (G-CSF), Hepatocyte Growth Factor (HGF), Hepsin, Human Epidermal Growth Factor Receptor 2 (HER-2), Immunoglobulin A (IgA), Immunoglobulin M (IgM), Insulin-like Growth Factor-Binding Protein 2 (IGFBP-2), Insulin-like Growth Factor Binding Protein 4 (IGFBP4), Insulin-like Growth Factor Binding Protein 5 (IGFBP5), Insulin-like Growth Factor Binding Protein 6 (IGFBP6), Intercellular Adhesion Molecule 1 (ICAM-1), Interferon gamma Induced Protein 10 (IP-10), Interferon-inducible T-cell alpha chemoattractant (ITAC), Interleukin-1 alpha (IL-1 alpha), Interleukin-1 receptor antagonist (IL-1ra), Interleukin-2 receptor alpha (IL-2 receptor alpha), Interleukin-6 receptor (IL-6r), Interleukin-6 receptor subunit beta (IL-6R beta), Interleukin-7 (IL-7), Interleukin-12 Subunit p40 (IL-12p40), Interleukin-15 (IL-15), Interleukin-16 (IL-16), Kallikrein 5, Kidney Injury Molecule-1 (KIM-1), Lactoylglutathione lyase (LGL), Latency-Associated Peptide of Transforming Growth Factor beta 1 (LAP TGF-b1), Lectin-Like Oxidized LDL Receptor 1 (LOX-1), Leptin, Macrophage Colony-Stimulating Factor 1 (M-CSF), Macrophage-Derived Chemokine (MDC), Macrophage inflammatory protein 3 beta (MIP-3 beta), Macrophage Migration Inhibitory Factor (MIF), Macrophage-Stimulating Protein (MSP), Maspin, Matrix Metalloproteinase-1 (MMP-1), Matrix Metalloproteinase-3 (MMP-3), Matrix Metalloproteinase-9—total (MMP-9, total), Mesothelin (MSLN), Monocyte Chemotactic Protein 1 (MCP-1), Monocyte Chemotactic Protein 2 (MCP-2), Monocyte Chemotactic Protein 4 (MCP-4), Myeloid Progenitor Inhibitory Factor 1 (MPIF-1), Myeloperoxidase (MPO), Myoglobin, Neuron-Specific Enolase (NSE), Neuronal Cell Adhesion Molecule (Nr-CAM), Neuropilin-1, Neutrophil Gelatinase-Associated Lipocalin (NGAL), Osteopontin, Osteoprotegerin (OPG), Plasminogen Activator Inhibitor 1 (PAI-1), Platelet-Derived Growth Factor BB (PDGF-BB), ProgesteroneProinsulin (Intact), Proinsulin (Total), Protein S100-A4 (S100-A4), Pulmonary and Activation-Regulated Chemokine (PARC), Receptor for advanced glycosylation end products (RAGE), Receptor tyrosine-protein kinase erbB-3 (ErbB3), Resistin, Serotransferrin (Transferrin), Serum Amyloid P-Component (SAP), Sortilin, Squamous Cell Carcinoma Antigen-1 (SCCA-1), Stem Cell Factor (SCF), Stromal cell-derived factor-1 (SDF-1), Superoxide Dismutase 1, soluble (SOD-1), T-Cell-Specific Protein RANTES (RANTES), Tenascin-C (TN-C), Tetranectin, Thrombomodulin (TM), Thrombospondin-1, Thyroxine-Binding Globulin (TBG), Tissue Inhibitor of Metalloproteinases 1 (TIMP-1), Tissue type Plasminogen activator (tPA), Transthyretin (TTR), Trefoil Factor 3 (TFF3), Tumor Necrosis Factor alpha (TNF-alpha), Tumor Necrosis Factor Receptor I (TNF R1), Tumor necrosis factor receptor 2 (TNFR2), Urokinase-type plasminogen activator receptor (uPAR), Vascular Cell Adhesion Molecule-1 (VCAM-1), Vascular Endothelial Growth Factor C (VEGF-C), Vascular Endothelial Growth Factor Receptor 2 (VEGFR-2), Vascular endothelial growth factor receptor 3 (VEGFR-3), Vitamin K-Dependent Protein S (VKDPS), Vitronectin, von Willebrand Factor (vWF) and YKL-40. See for example, Table 10a and Table 10b.

In some embodiments, SAT biomarkers can include but are not limited to B Lymphocyte Chemoattractant (BLC), Brain-Derived Neurotrophic Factor (BDNF), CD40 Ligand (CD40-L), Chromogranin-A (CgA), Creatine Kinase-MB (CK-MB), Eotaxin-1, Epidermal Growth Factor (EGF), Epithelial-Derived Neutrophil-Activating Protein 78 (ENA-78), Glucagon-like Peptide 1, total (GLP-1 total), Hepatocyte Growth Factor (HGF), Interleukin-12 Subunit p40 (IL-12p40), Kidney Injury Molecule-1 (KIM-1), Lactoylglutathione lyase (LGL), Latency-Associated Peptide of Transforming Growth Factor beta 1 (LAP TGF-b1), Macrophage Colony-Stimulating Factor 1 (M-CSF), Matrix Metalloproteinase-1 (MMP-1), Matrix Metalloproteinase-9—total (MMP-9, total), Monocyte Chemotactic Protein 4 (MCP-4), Myeloperoxidase (MPO), Myoglobin, Neuron-Specific Enolase (NSE), Plasminogen Activator Inhibitor 1 (PAI-1), Platelet-Derived Growth Factor BB (PDGF-BB), Proinsulin (Total), Protein S100-A4 (S100-A4), Receptor tyrosine-protein kinase erbB-3 (ErbB3), Squamous Cell Carcinoma Antigen-1 (SCCA-1), T-Cell-Specific Protein RANTES (RANTES), Thrombospondin-1, Tumor Necrosis Factor alpha (TNF-alpha) and Vascular Endothelial Growth Factor C (VEGF-C). See for example, Table 11.

In some embodiments, SAT biomarkers can include but are not limited to Brain-Derived Neurotrophic Factor (BDNF), Creatine Kinase-MB (CK-MB), Epidermal Growth Factor (EGF), Epithelial-Derived Neutrophil-Activating Protein 78 (ENA-78), Hepatocyte Growth Factor (HGF), Latency-Associated Peptide of Transforming Growth Factor beta 1 (LAP TGF-b1), Matrix Metalloproteinase-1 (MMP-1), Matrix Metalloproteinase-9-—total (MMP-9, total), Myoglobin, Neuron-Specific Enolase (NSE), Plasminogen Activator Inhibitor 1 (PAI-1), Platelet-Derived Growth Factor BB (PDGF-BB), Receptor tyrosine-protein kinase erbB-3 (ErbB3), T-Cell-Specific Protein RANTES (RANTES), Thrombospondin-1, and Vascular Endothelial Growth Factor C (VEGF-C). See for example, Table 12.

In some embodiments, SAT biomarkers can include but are not limited to Brain-Derived Neurotrophic Factor (BDNF), Creatine Kinase-MB (CK-MB), Epidermal Growth Factor (EGF), Epithelial-Derived Neutrophil-Activating Protein 78 (ENA-78), Hepatocyte Growth Factor (HGF), Latency-Associated Peptide of Transforming Growth Factor beta 1 (LAP TGF-b1), Matrix Metalloproteinase-1 (MMP-1), Matrix Metalloproteinase-9—total (MMP-9, total), Myoglobin, Plasminogen Activator Inhibitor 1 (PAI-1), Platelet-Derived Growth Factor BB (PDGF-BB), Receptor tyrosine-protein kinase erbB-3 (ErbB3), T-Cell-Specific Protein RANTES (RANTES), Thrombospondin-1 and Vascular Endothelial Growth Factor C (VEGF-C). See for example, Table 13.

In some embodiments, SAT biomarkers can include but are not limited to Chromogranin-A (CgA), Epithelial-Derived Neutrophil-Activating Protein 78 (ENA-78) Lactoylglutathione lyase (LGL), Latency-Associated Peptide of Transforming Growth Factor beta 1 (LAP TGF-b1), Macrophage Colony-Stimulating Factor 1 (M-CSF), Matrix Metalloproteinase-9, total (MMP-9, total), Myoglobin, Neuronal Cell Adhesion Molecule (Nr-CAM), Plasminogen Activator Inhibitor 1 (PAI-1), Receptor tyrosine-protein kinase erbB-3 (ErbB3), and Vascular Endothelial Growth Factor C (VEGF-C). See for example, Table 14. In some embodiments, at least one, at least two, at least three, at least four, at least five, at least six, at least seven, at least eight, at least nine, at least ten, at least eleven or more of the biomarkers provides a level pattern that indicates efficacy of the treatment. In some embodiments, Chromogranin-A (CgA), Epithelial-Derived Neutrophil-Activating Protein 78 (ENA-78) Lactoylglutathione lyase (LGL), Latency-Associated Peptide of Transforming Growth Factor beta 1 (LAP TGF-b1), Myoglobin, Neuronal Cell Adhesion Molecule (Nr-CAM), and/or Plasminogen Activator Inhibitor 1 (PAI-1) provides a “normalization” level pattern after the treatment. In some embodiments, Macrophage Colony-Stimulating Factor 1 (M-CSF), Matrix Metalloproteinase-9, total (MMP-9, total), Receptor tyrosine-protein kinase erbB-3 (ErbB3), and/or Vascular Endothelial Growth Factor C (VEGF-C) provides a “stabilization” level pattern after the treatment.

In some embodiments, SAT biomarkers can include but are not limited to Epithelial-Derived Neutrophil-Activating Protein 78 (ENA-78), CD40 Ligand (CD40-L), Interleukin-3 (IL-3), Interleukin-5 (IL-5), Interleukin-7 (IL-7), Interleukin-8 (IL-8), Latency-Associated Peptide of Transforming Growth Factor beta 1 (LAP TGF-b1), Macrophage Colony-Stimulating Factor 1 (M-CSF), Macrophage Inflammatory Protein-3 alpha (MIP-3 alpha), Myeloperoxidase, T-Cell-Specific Protein RANTES (RANTES), Vascular Endothelial Growth Factor C (VEGF-C), Agouti-Related Protein (AgRP), Thrombospondin-1, Platelet-Derived Growth Factor BB (PDGF-BB), Matrix Metalloproteinase-1 (MMP-1), Matrix Metalloproteinase-3 (MMP-3) and Matrix Metalloproteinase-9 (MMP-9), Brain-Derived Neurotrophic Factor (BDNF), Chromogranin-A (CgA), Receptor tyrosine-protein kinase erbB-3 (ErbB-3), Neuron-Specific Enolase (NSE), Neural cell adhesion molecule (Nr-CAM), Epidermal Growth Factor (EGF), Fibroblast Growth Factor 4 (FGF-4), Kallikrein 5, Plasminogen Activator Inhibitor 1 (PAI-1), Serum Glutamic Oxaloacetic Transaminase (SGOT), Creatine Kinase-MB (CK-MB), Myoglobin, N-terminal prohormone of brain natriuretic peptide (NT proBNP), Protein S100-A4 (S100-A4), Protein S100-A6 (S100-A6), Insulin-like Growth Factor Binding Protein 6 (IGFBP-6), Thyroglobulin, Amphiregulin and Cortisol.

In some embodiments, SAT biomarkers are associated with muscle inflammation and fibrosis. Muscle inflammation and fibrosis associated biomarkers can include but are not limited to Epithelial-Derived Neutrophil-Activating Protein 78 (ENA-78), CD40 Ligand (CD40-L), Interleukin-3 (IL-3), Interleukin-5 (IL-5), Interleukin-7 (IL-7), Interleukin-8 (IL-8), Latency-Associated Peptide of Transforming Growth Factor beta 1 (LAP TGF-b1), Macrophage Colony-Stimulating Factor 1 (M-CSF), Macrophage Inflammatory Protein-3 alpha (MIP-1 alpha), Myeloperoxidase, T-Cell-Specific Protein RANTES (RANTES), Vascular Endothelial Growth Factor C (VEGF-C), Agouti-Related Protein (AgRP), Thrombospondin-1, Platelet-Derived Growth Factor BB (PDGF-BB), Matrix Metalloproteinase-1 (MMP-1), Matrix Metalloproteinase-3 (MMP-3) and Matrix Metalloproteinase-9 (MMP-9). See for example, Table 2. In some embodiments, the SAT biomarkers are associated with muscle inflammation and fibrosis. Muscle inflammation and fibrosis associated biomarkers can include but are not limited to CD40 Ligand (CD40-L), Interleukin-3 (IL-3), Interleukin-5 (IL-5), Interleukin-7 (IL-7), Interleukin-8 (IL-8), Latency-Associated Peptide of Transforming Growth Factor beta 1 (LAP TGF-b1), T-Cell-Specific Protein RANTES (RANTES), Vascular Endothelial Growth Factor C (VEGF-C), Agouti-Related Protein (AgRP), Thrombospondin-1, Platelet-Derived Growth Factor BB (PDGF-BB) and Matrix Metalloproteinase-9 (MMP-9). See for example, Table 2 (biomarkers highlighted in red and bolded).

In some embodiments, the SAT biomarkers are associated with muscle and nerve development. Muscle and nerve associated biomarkers can include but are not limited to Brain-Derived Neurotrophic Factor (BDNF), Chromogranin-A (CgA), Receptor tyrosine-protein kinase erbB-3 (ErbB-3), Neuron-Specific Enolase (NSE), Neural cell adhesion molecule (Nr-CAM), Epidermal Growth Factor (EG), Fibroblast Growth Factor 4 (FGF-4), Kallikrein 5 and Plasminogen Activator Inhibitor 1 (PAI-1). See for example, Table 3. In some embodiments, the muscle and nerve associated biomarkers and include but are not limited to Brain-Derived Neurotrophic Factor (BDNF), Chromogranin-A (CgA), Receptor tyrosine-protein kinase erbB-3 (ErbB-3), Neuron-Specific Enolase (NSE), Fibroblast Growth Factor 4 (FGF-4), and Plasminogen Activator Inhibitor 1 (PAI-1). See for example, Table 3 (biomarkers highlighted in red and bolded).

In some embodiments, the SAT biomarkers are associated with muscle damage. Muscle damage associated biomarkers can include but are not limited to Serum Glutamic Oxaloacetic Transaminase (SGOT), Creatine Kinase-MB (CK-MB) and Myoglobin. See for example, Table 4. In some embodiments, the muscle damage associated biomarkers include but are not limited to myoglobin. See for example, Table 4 biomarkers (biomarkers highlighted in red and bolded).

In some embodiments, the SAT biomarkers are other biomarkers, such as biomarkers associated with cardiovascular risk, Calcium binding protein, tissue morphology, EGF and TGF-a, the stress response and suppression of the immune system. Such other biomarkers can include but are not limited to N-terminal prohormone of brain natriuretic peptide, Protein S100-A4 (S100-A4), Protein S100-A6 (S100-A6), Insulin-like Growth Factor Binding Protein 6 (IGFBP-6), Thyroglobulin, Amphiregulin and Cortisol. See for example, Table 5. In some embodiments, other biomarkers include but are not limited to Insulin-like Growth Factor Binding Protein 6. See for example, Table 5 (biomarkers highlighted in red and bolded).

In some embodiments, the level of one or more than one SAT biomarkers is determined or detected. The levels of individual SAT biomarkers can be determined using a variety of methods know to those of skill in the art, including but not limited to those described in the present application. Additionally, the levels of more than one SAT biomarker can be determined in order to generate a composite of the level of more than one SAT biomarker. In some embodiments, the methods of the present invention comprise determining a composite level of a panel of selected SAT biomarkers. In some embodiments, the methods comprise determining a composite level of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more selected SAT biomarkers. The composite can contain any of the SAT biomarkers described by the present application.

A composite of the level of SAT biomarkers can include any collection of information regarding the level of more than one SAT biomarker. Information can include nucleic acid or protein information, or a combination of information regarding both nucleic acid and/or protein levels. Generally, the composite includes information regarding the increase or decrease in the level of selected SAT biomarkers.

In some embodiments, the information in the composite can include index values based on the increase or decrease in the biomarker level. For example, the level of each SAT biomarker in a group of SAT biomarkers can be assigned an index value based on the increase or the decrease in the SAT biomarker. In some embodiments, the larger the increase or decrease, the larger the index value. In some embodiments, the index values can be compiled as group to generate a composite. In some embodiments, the composite is compared to a predetermined standard. In some embodiments, comparison of the composite to a predetermined standard is indicative of treatment efficacy of treatment with the therapeutic entity. In some embodiments, comparison of the composite to a predetermined standard is predictive of treatment efficacy of treatment with the therapeutic entity. In some embodiments, comparison of the composite to a predetermined standard can be used to modify the treatment regimen of treatment with a therapeutic entity. The composite can also contain information regarding the increase of selected SAT biomarkers and the decrease of other selected SAT biomarkers. The composite can also contain information regarding the change in biomarker levels before and after treatment with a therapeutic entity and such information can be used to modify the treatment regimen of treatment with the therapeutic entity. In some embodiments treatment efficacy is determined by determining the increase of selected SAT biomarkers and the decrease of other selected biomarkers, wherein the biomarkers that increase are not the same as the biomarkers that decrease.

The SAT biomarkers for the methods of the present invention can include both the nucleic acid and protein forms of the biomarkers. Methods for detecting the levels of nucleic acids and proteins are well known in the art and any standard methods for detection of nucleic acid or protein levels can be employed with the methods of the present invention and used for detecting the levels of SAT biomarkers. The SAT biomarkers can also include small nucleotide polymorphisms (SNPs).

Methods for detecting the levels of nucleic acids, such as RNA or DNA have been well described and are well known to those of skill in the art. Methods for detecting RNA can include but are not limited to RT-PCR, northern blot analyses, gene expression analyses, microarray analyses, gene expression chip analyses, hybridization techniques (including FISH), expression beadchip arrays, and chromatography as well as any other techniques known in the art. Methods for detecting DNA can include but are not limited to PCR, real-time PCR, digital PCR, hybridization (including FISH), microarray analyses, SNP detection assays, SNP genotyping assays and chromatography as well as any other techniques known in the art.

Methods for detecting proteins and polypeptides can include but are not limited to spectrophotometric determination of protein concentration, quantitative amino acid analysis, protein concentration assays, chromatography assays, western blot analyses, gel electrophoresis, (followed by staining procedures including but not limited to Coomassie Blue, Silver stain, Syber Green, Syber Gold), hybridization, multiplex cytokine assays, immunoassays, ELISA, bicinchoninic acid (BCA) protein assays, Bradford protein assays, and Lowry protein assays as well as any other techniques known in the art. Protein detection can also include detecting the levels of stable or active proteins and methods such as kinetic assays, kinase assays, enzyme assays and post-translation modification assays (for example, assays for determining phosphorylation and glycosylation state) can also be employed.

Methods for quantitating nucleic acid and protein levels have been well described. Methods can include traditional methods, such as western blot quantization and immunoassays as well as computer based methods, such as microarray assay or genechip assay analyses, for analyzing SAT biomarker levels. Immunoassays can include for example, the Human Discovery 250+ Immunoassay from Myriad RBM (commercially available from Myriad RBM, Texas, USA). These analyses can include protein level analyses as well as gene expression level analyses. These standard methods known in the art can be employed to determine whether the level of a SAT biomarker has increased or decreased. An increase or decrease in the level of the SAT biomarker can be based on a comparison of the level of the biomarker with a predetermined standard level or by comparison of the biomarker level after treatment with the therapeutic entity to the SAT biomarker level before treatment with the therapeutic entity, e.g. a sialic acid deficiency treatment. By way of non-limiting examples, Tables 2-14 show relative changes for exemplary SAT biomarkers contemplated for use with methods of the present invention.

In some embodiments detection of the level of the SAT biomarker can include detection of the level of proteins, nucleic acids, nucleic acid presence or absence (gene presence or absence), gene expression or SNP presence or absence. In other embodiments the biomarker can include SNPs. In some embodiments detection of the level of the one or more SAT biomarker can include detection of the presence or absence of one or more SNPs in the SAT biomarker.

In some embodiments, the level of the one or more SAT biomarkers is determined prior to treatment with the therapeutic entity and the level is then determined again after treatment with the therapeutic entity. In other embodiments the level of the one or more SAT biomarkers is determined after treatment with the therapeutic entity and compared to a predetermined standard level. In yet other embodiments the level of the one or more SAT biomarkers is measured prior to treatment with the therapeutic entity and compared to a predetermined standard level.

As used herein, the term “predetermined standard level” or “predetermined activity profiles” refers to standardized data or data set representing the average, representative features or characteristics of one or more biomarkers in a specific population. Such features or characteristics include, but are not limited to, transcript abundance, transcript stability, transcription rate, translation rate, post-translation modification, protein abundance, protein stability, and/or protein enzymatic activity, etc. In some embodiments, the specific population of subjects are consisting of about 5, about 10, about 20, about 50, about 100, about 200, about 300, about 400, about 500, about 1000, about 5000, about 10K, or more individual subjects. The predetermined activity profile can be a standardized data or data set collected before, during, or after the specific population of subjects has been all exposed to a drug. In some embodiments, the specific population is consisting of clinically normal subjects. As used herein, the term “clinically normal subject” refers to a subject without, or substantially without the symptoms associated with sialic acid deficiencies. Predetermined standard levels of SAT biomarkers can be defined using a variety of methods known to those of skill in the art. Generally, standard levels for a biomarker are determined by determining the level of a SAT biomarker in a sufficiently large number of samples obtained from normal, healthy control subjects, for example Table 2 which describes clinically normal SAT biomarker levels. Further, standard level information can be obtained from publically available databases, as well as other sources. (See, e.g., Bunk, D. M., Clin. Biochem. Rev., 28(4):131-137 (2007); Suraj Peril, et al., Genome Res. 13: 2363-2371 (2003); Remington: The Science and Practice of Pharmacy, Twenty First Edition (2005).) For example, in some embodiments, an increase or decrease of the level of one or more SAT biomarkers in a sample obtained from a subject treated with a sialic acid deficiency treatment is determined by comparing the level of one or more SAT biomarkers to a predetermined standard level, such as for example a level as described in Table 2.

As used herein, a subject is “responsive” to a drug for treating sialic acid deficiencies when the level of one or more of the biomarkers of the present invention increases or decreases toward a pre-determined standard level when the subject is exposed to a the drug, or when the drug modifies the speed of level changes of one or more biomarkers of the present invention compared to a placebo.

For methods related to detection, quantitation and comparison of biomarker levels, see, e.g., Current Protocols in Molecular Biology, Ed. Ausubel, Frederick M. (2010); Current Protocols in Protein Science Last, Ed. Coligan, John E., et al. (2010); Current Protocols in Nucleic Acid Chemistry, Ed. Egli, Martin (2010); Current Protocols in Bioinformatics, Ed. Baxevanis, Andreas D. (2010); and Molecular Cloning: A Laboratory Manual, Third Edition, Sambrook, Joseph (2001), all of which are incorporated herein by reference in their entirety.

In certain embodiments, when measuring biomarkers or other indicators of treatment, an “increased” or “decreased” amount or level may include a “statistically significant” amount. A result is typically referred to as statistically significant if it is unlikely to have occurred by chance. The significance level of a test or result relates traditionally to the amount of evidence required to accept that an event is unlikely to have arisen by chance. In certain cases, statistical significance may be defined as the probability of making a decision to reject the null hypothesis when the null hypothesis is actually true (a decision known as a Type I error, or “false positive determination”). This decision is often made using the p-value: if the p-value is less than the significance level, then the null hypothesis is rejected. The smaller the p-value, the more significant the result. Bayes factors may also be utilized to determine statistical significance (see, e.g., Goodman S., Ann Intern Med. 130:1005-13, 1999). In some embodiments, an “increased” or “decreased” amount or level is about 1.1×, 1.2×, 1.3×, 1.4×, 1.5×, 2×, 2.5×, 3×, 3.5×, 4×, 4.5×, 5×, 6×, 7×, 8×, 9×, 10×, 15×, 20×, 25×, 30×, 40×, or 50× more or less the amount of a predetermined standard, or the amount of a determined time point relative to a previous or earlier timepoint.

Methods for obtaining biological samples are well known in the art and any standard methods for obtaining biological samples can be employed. Biological samples that find use with the methods of the present invention include but are not limited to blood (including but not limited to serum, blood, plasma, whole blood and derivatives thereof), skin, hair, hair follicles, saliva, oral mucous, vaginal mucous, sweat, tears, epithelial tissues, urine, semen, seminal fluid, seminal plasma, prostatic fluid, pre-ejaculatory fluid (Cowper's fluid), excreta, biopsy, ascites, cerebrospinal fluid, lymph, and tissue extract sample or biopsy samples. (See, e.g., Clinical Proteomics: Methods and Protocols, Vol. 428 in Methods in Molecular Biology, Ed. Antonia Vlahou (2008).) In some embodiments, the biological sample is selected from blood (including but not limited to serum, blood, plasma, whole blood and derivatives thereof), skin, hair, hair follicles, saliva, oral mucous, vaginal mucous, sweat, tears, epithelial tissues, urine, semen, seminal fluid, seminal plasma, prostatic fluid, pre-ejaculatory fluid (Cowper's fluid), excreta, biopsy, ascites, cerebrospinal fluid, lymph, and tissue extract sample or biopsy sample. In some embodiments, the biological sample is a blood sample.

According to the methods of the present invention, the term “subject,” and variants thereof as used herein, includes any subject that has, is suspected of having, or is at risk for having a sialic acid deficiency disease or condition. Suitable subjects (or patients) include mammals, such as laboratory animals (e.g., mouse, rat, rabbit, guinea pig), farm animals, and domestic animals or pets (e.g., cat, dog). Non-human primates and, preferably, human patients, are included. A subject “at risk” may or may not have detectable disease, and may or may not have displayed detectable disease prior to the diagnostic or treatment methods described herein. “At risk” denotes that a subject has one or more so-called risk factors, which are measurable parameters that correlate with development of a condition of sialic acid deficiency, which are described herein. A subject having one or more of these risk factors has a higher probability of developing a sialic acid deficiency than a subject without these risk factor(s). One example of such a risk factor is an increase or decrease in a SAT biomarker as compared to a “clinically normal” sample.

The term “effective amount” refers to the amount of one or more compounds that renders a desired treatment outcome. An effective amount may be comprised within one or more doses, i.e., a single dose or multiple doses may be required to achieve the desired treatment endpoint.

The term “therapeutically effective amount” as used herein, refers to the level or amount of one or more agents needed to treat a condition, or reduce or prevent injury or damage, optionally without causing significant negative or adverse side effects. For instance, a therapeutically effective amount includes an amount of a pharmaceutical formulation including for example one or more compounds in the sialic acid biosynthesis pathway sufficient to produce a desired therapeutic outcome (e.g., reduction of severity of a disease or condition).

A “prophylactically effective amount” refers to an amount of an agent (e.g., a pharmaceutical formulation including one or more compounds in the sialic acid biosynthesis pathway) sufficient to prevent or reduce severity of a future disease or condition when administered to a subject who is susceptible and/or who may develop a disease or condition.

By “pharmaceutically acceptable” is meant a material that is not biologically or otherwise undesirable, i.e., the material may be incorporated into a pharmaceutical composition administered to a patient without causing any significant undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration.

The term “disorder” or “disease” used interchangeably herein, refers to any alteration in the state of the body or one of its organs and/or tissues, interrupting or disturbing the performance of organ function and/or tissue function (e.g., causes organ dysfunction) and/or causing a symptom such as discomfort, dysfunction, distress, or even death to a subject afflicted with the disease.

The term “subject”, “individual” or “patient” refers to an animal, for example, a mammal and includes, but is not limited to, human, bovine, horse, feline, canine, rodent, or primate. In some embodiments, the individual is a human.

The term “derivative” as used herein includes derivatives, analogs, prodrugs, and unnatural precursors.

The terms “treating” and “treatment” as used herein refer to an approach for obtaining beneficial or desired results including clinical results, and may include even minimal changes or improvements in one or more measurable markers of the disease or condition being treated. A treatment is usually effective to reduce at least one symptom of a condition, disease, disorder, injury or damage. Exemplary markers of clinical improvement will be apparent to persons skilled in the art. Examples include, but are not limited to, one or more of the following: decreasing the severity and/or frequency one or more symptoms resulting from the disease, diminishing the extent of the disease, stabilizing the disease (e.g., preventing or delaying the worsening of the disease), delay or slowing the progression of the disease, ameliorating the disease state, increasing production of sialic acid, the sialylation precursor CMP-sialic acid (e.g., increasing intracellular production of sialic acid) and restoring the level of sialylation in muscle and other proteins, decreasing the dose of one or more other medications required to treat the disease, and/or increasing the quality of life.

“Prophylaxis,” “prophylactic treatment,” or “preventive treatment” refers to preventing or reducing the occurrence or severity of one or more symptoms and/or their underlying cause, for example, prevention of a disease or condition in a subject susceptible to developing a disease or condition (e.g., at a higher risk, as a result of genetic predisposition, environmental factors, predisposing diseases or disorders, or the like). Prophylaxis includes prophylaxis of HIBM myopathy in which chronic disease changes in the muscles are irreversible, and for which animal model data suggests that prophylactic treatment prior to such irreversible damage confers a significant treatment benefit.

The phrase “determining the treatment efficacy” or “determining the efficacy of treatment” and variants thereof can include any methods for determining that a treatment is providing a benefit to a subject, including for example clinical results or minimal changes or improvements as discussed above. The term “treatment efficacy” and variants thereof are generally indicated by alleviation of one or more signs or symptoms associated with the disease and can be readily determined by one skilled in the art. “Treatment efficacy” may also refer to the prevention or amelioration of signs and symptoms of toxicities typically associated with standard or non-standard treatments of a disease. Determination of treatment efficacy is usually indication and disease specific and can include any methods known or available in the art for determining that a treatment is providing a beneficial effect to a subject. For example, evidence of treatment efficacy can include but is not limited to general improvements in the overall health of the subject, such as but not limited to enhancement of patient life quality, increase in predicted subject survival rate, decrease in depression, decreasing the severity and/or frequency one or more symptoms resulting from the disease, diminishing the extent of the disease, stabilizing the disease (e.g., preventing or delaying the worsening of the disease), delay or slowing the progression of the disease, ameliorating the disease state, increasing production of sialic acid, the sialylation precursor CMP-sialic acid (e.g., increasing intracellular production of sialic acid) and restoring the level of sialylation in muscle and other proteins, decreasing the dose of one or more other medications required to treat the disease, and/or increasing the quality of life (increase in remission time). (See, e.g., Physicians' Desk Reference (2010).)

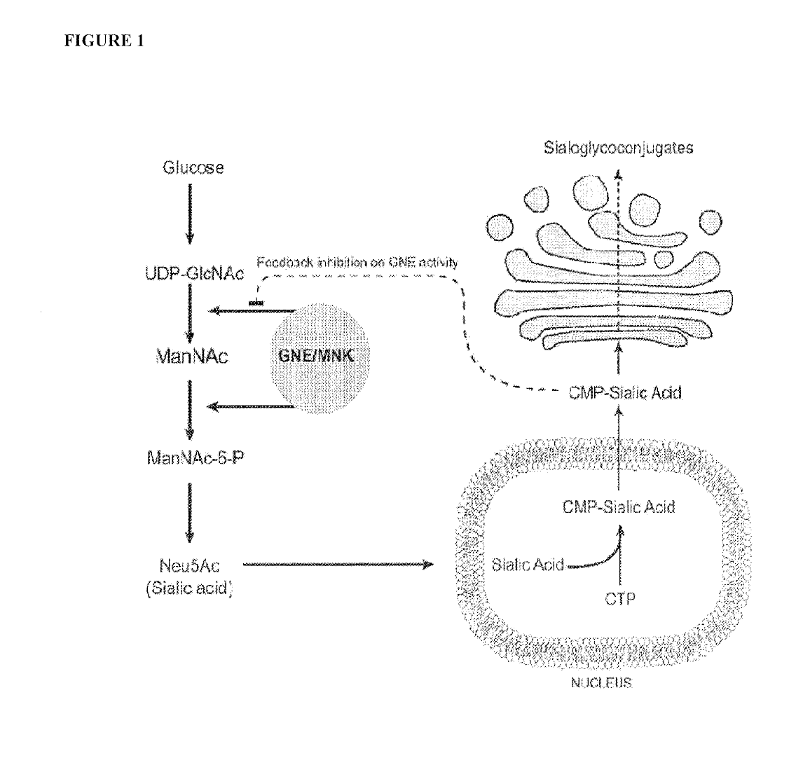

Certain embodiments include treatment of a condition of sialic acid deficiency, and related therapeutic agents and pharmaceutical compositions/formulations. Such treatments include but are not limited to replacement therapies, which typically achieve increased sialic acid levels by administering an agent that directly or indirectly increases one or more components of the sialic acid biosynthesis pathway (see, e.g., FIG. 1).

Also included as part of such replacement therapies are gene therapies. Such gene therapies can incorporate one or more genes involved directly or indirectly in the sialic acid biosynthesis pathway. Exemplary components of the sialic acid biosynthesis pathway that can be used as part of gene therapies include mannosamine, N-acetyl mannosamine (ManNAc), ManNac-6-phosphate (ManNAc-6-P), UDP-GlcNAc, N-acetylneuraminic acid (NeuAc), NeuAc-9-phosphate (NeuAc-9-P), sialic acid (i.e., 5-N-acetylneuraminic acid), and CMP-sialic acid. Hence, certain treatments include the direct administration of one or more of these components as compounds, or as derivatives or pharmaceutically acceptable salts thereof, including extended release formulations of such compounds (see, e.g., U.S. Application No. 61/363,995; and PCT/US2011/043910, each of which is incorporated by reference in its entirety). The term “derivative” as used herein includes derivatives, analogs, prodrugs, and unnatural precursors of a given compound. In specific embodiments, the compound in the sialic acid biosynthesis pathway or a derivative thereof does not include glucose or a pharmaceutically acceptable salt thereof.

As one example, the one or more compounds in the sialic acid biosynthesis pathway or derivative thereof include ManNAc or a derivative thereof (see, e.g., U.S. Application No. 2010/0249047 and WO 200/8150477, which are incorporated by reference in their entireties). Structures of such ManNAc and derivatives thereof include, but are not limited to, those defined by the formula below:

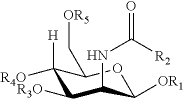

wherein: R₁, R₃, R₄, or R₅ is hydrogen, lower alkanoyl, carboxylate or lower alkyl; and R₂ is lower alkyl, lower alkanoylalkyl, lower alkyl alkanoyloxy.

The term lower alkyl refers to (C₁-C₆)alkyl. A lower alkyl includes methyl, ethyl, propyl, isopropyl, butyl, iso-butyl, sec-butyl, pentyl, 3-pentyl, hexyl, (C₃-C₆)cycloalkyl (e.g., cyclopropyl, cyclobutyl, cyclopentyl, or cyclohexyl), (C₃-C₆)cycloalkyl(C₁-C₆)alkyl (e.g., cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl), (C₁-C₆)alkoxy (e.g., methoxy, ethoxy, propoxy, isopropoxy, butoxy, iso-butoxy, sec-butoxy, pentoxy, 3-pentoxy, or hexyloxy) (C₂-C₆)alkenyl (e.g., vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl), (C₂-C₆)alkynyl (e.g., ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl), (C₁-C₆)alkanoyl (e.g., acetyl, propanoyl or butanoyl), halo(C₁-C₆)alkyl (e.g., iodomethyl, bromomethyl, chloromethyl, fluoromethyl, trifluoromethyl, 2-chloroethyl, 2-fluoroethyl, 2,2,2-trifluoroethyl, or pentafluoroethyl), hydroxy(C₁-C₆)alkyl (e.g., hydroxymethyl, 1-hydroxyethyl, 2-hydroxyethyl, 1-hydroxypropyl, 2-hydroxypropyl, 3-hydroxypropyl, 1-hydroxy butyl, 4-hydroxybutyl, 1-hydroxypentyl, 5-hydroxypentyl, 1-hydroxyhexyl, or 6-hydroxyhexyl), C₆)alkoxycarbonyl (e.g., methoxycarbonyl, ethoxycarbonyl, propoxycarbonyl, isopropoxycarbonyl, butoxycarbonyl, pentoxycarbonyl, or hexyloxycarbonyl), (C C₆)alkylthio (e.g., methylthio, ethylthio, propylthio, isopropylthio, butylthio, isobutylthio, pentylthio, or hexylthio), and/or (C₂-C₆)alkanoyloxy (e.g., acetoxy, propanoyloxy, butanoyloxy, isobutanoyloxy, pentanoyloxy, or hexanoyloxy).

In some embodiments, R₂ is methyl, and R₁, R₃, R₄, and R₅ is hydrogen. In some embodiments, the ManNAc or derivative thereof is N-acetyl mannosamine (ManNAc). In some embodiments, the ManNAc or derivative thereof is N-levulinoylmannosamine (ManLev) or N-azidoacetylmannosamine (ManNAz).

In some embodiments, the one or more compounds in the sialic acid biosynthesis pathway or derivative thereof include N-acetylneuraminic acid (NeuAc) or a derivative thereof. Structures of such NeuAc or derivatives thereof include, but are not limited to, those defined by the formula below:

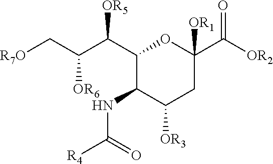

wherein each R₁, R₂, R₃, R₅, R₆, or R₇ is independently hydrogen, lower alkanoyl, carboxylate or lower alkyl; and R₄ is lower alkyl, lower alkanoylalkyl or lower alkyl alkanoyloxy.

In some embodiments, the one or more compounds in the sialic acid biosynthesis pathway or derivative thereof include sialic acid or a derivative thereof, including for example both N- or O-substituted derivatives of neuraminic acid such as N-acetylneuraminic acid (Neu5Ac or NANA). In some embodiments, the sialic acid or derivative thereof is sialic acid. In some embodiments, the sialic acid or derivative thereof is a sialic acid analog such as N-levulinoyl sialic acid (SiaLev), N-azidoacetyl sialic acid (SiaNAz). In some embodiments, the sialic acid or derivative thereof is bound as a glycoconjugate. In some embodiments, the sialic acid or derivative thereof is an unnatural precursor such as sialylactose. In some embodiments the sialic acid or derivative thereof is conjugated to an immunoglobulin. Specific embodiments include a sialic acid extended release formulation (see, e.g., U.S. Application No. 61/363,995; and PCT/US2011/043910). In specific embodiments, the extended release formulation is a formulation of Table 1, below.

TABLE 1
Quantitative Formula for Sialic Acid 325 mg and 500 mg sustained
release Tablets Prototypes.

| | | mg/Tablet | |
|---|---|---|---|
| | | ProCR | |
| Ingredient | Vendor | Hypromellose | % w/w |
| Sialic Acid (N- | Food and | 325.0 | 43.3 |
| Acetylneuraminic | BioResearch | | |
| acid) | Center, Inc | | |
| Hypromellose, | Colorcon | 191.3 | 25.5 |
| Type 2208 | | | |
| (Methocel ® K100 | | | |
| M Premium CR) | | | |
| Polyethylene Oxide | | — | 25.5 |
| WSR (Polyox) | | | |
| Sodium Alginate | FMC | 159.0 | 21.2 |
| (Protanal ® LF | Biopolymer | | |
| 120M) | | | |
| Carrageenan | FMC | 31.5 | 4.2 |
| (Viscarin GP-209) | Biopolymer | | |
| Microcrystallline | JRS Pharma | 39.8 | 5.3 |
| Cellulose and | | | |
| Colloidal Sillicon | | | |
| Dioxide (ProSolv ® | | | |
| SMCC HD 90) | | | |
| Magnesium | Mallinckrodt | 3.8 | 0.5 |
| Stearate | | | |
| (HyQual ®), | | | |
| Vegatable Source | | | |
| Product Code 2257 | | | |

| | | |
|---|---|---|
| Total for 325 mg Strength | 750.4 | 100 |
| Total for 500 mg Strength | 1154.5 | 100 |

In one variation, the one or more compounds in the sialic acid biosynthesis pathway or derivative thereof is an ester of a compound in the sialic acid biosynthesis pathway. In one aspect, the one or more compounds in the sialic acid biosynthesis pathway or derivative thereof is an ester of sialic acid or MaNAc. In a particular variation, the one or more compounds in the sialic acid biosynthesis pathway or derivative thereof is an ester of sialic acid. In one aspect, the one or more compounds in the sialic acid biosynthesis pathway or derivative thereof is a prodrug of sialic acid. See also WO 2010/131712, published Nov. 18, 2010, for derivatives of compounds in the sialic acid biosynthesis pathway, which is incorporated herein by reference in its entirety and specifically with respect to compounds (e.g., derivatives of compounds in the sialic acid biosynthesis pathway) detailed therein.

In one aspect, a derivative of one or more compounds in the sialic acid biosynthesis pathway (e.g., a derivative of sialic acid or MaNAc) is an effective substrate replacement for sialic acid, such as in an subject who has or is suspected of having a condition of sialic acid deficiency. A derivative of one or more compounds in the sialic acid biosynthesis pathway (e.g., a derivative of sialic acid or MaNAc), or an extended release formulation comprising a derivative of one or more compounds in the sialic acid biosynthesis pathway (e.g., a derivative of sialic acid or MaNAc) may exhibit any one or more of the following characteristics: (i) capable of delivering to an individual in need thereof a therapeutically effective amount of one or more compounds in the sialic acid pathway or derivatives thereof over a period of greater than about any of 1, 2, 3, 4, 5, 6, 7, 8, 9, 10, 11, 12, 13, 14, 15, 16, 17, 18, 19, 20, 21, 22, 23, or 24 hours; (ii) capable of delivering to an individual in need thereof a substantially constant therapeutically effective amount of one or more compounds in the sialic acid pathway or derivatives thereof over a period of greater than about any of 1, 2, 3, 4, 5, 6, 7, 8, 9, 10, 11, 12, 13, 14, 15, 16, 17, 18, 19, 20, 21, 22, 23, or 24 hours; (iii) capable of delivering to an individual in need thereof a therapeutically effective amount of one or more compounds in the sialic acid pathway or derivatives thereof with a Tₘₐₓ of between about any of 2-4 hours, 3-4 hours, 6-8 hours, 6-12 hours, 6-15 hours, 12-18 hours, or 18-24 hours; (iv) capable of delivering to an individual in need thereof a therapeutically effective amount of one or more compounds in the sialic acid pathway or derivatives thereof with a Cₘₐₓ of about 0.5-100 μg/mL; (v) capable of delivering to an individual in need thereof a therapeutically effective amount of one or more compounds in the sialic acid pathway or derivatives thereof with a trough level of about 0.1-20 ng/mL; (vi) capable of delivering to an individual in need thereof a therapeutically effective amount of one or more compounds in the sialic acid pathway or derivatives thereof with less than about any of 10%, 20%, 30%, 40%, 50%, 60%, or 70% excreted after one hour; (vii) capable of delivering to an individual in need thereof between about any of 0.01-750 mg/kg/day, 0.5-500 mg/kg/day, 1-250 mg/kg/day, 2.5-100 mg/kg/day, or 5-50 mg/kg/day of one or more compounds in the sialic acid pathway or derivatives thereof or a pharmaceutically acceptable salt of the foregoing; (viii) capable of delivering to an individual in need thereof between about any of 0.01-750 mg/kg/day, 0.5-500 mg/kg/day, 1-250 mg/kg/day, 2.5-100 mg/kg/day, or 5-50 mg/kg/day of one or more compounds in the sialic acid pathway or derivatives thereof or a pharmaceutically acceptable salt of the foregoing; (ix) has an absolute bioavailability of about 1 to about 50%; (x) has a bioavailability based on sialic acid levels in the urine of about 0.5 to about 100%; and (xi) has a mean residence time (MRT) of at least about 3.5 hours.

As noted above, gene replacement therapy is also contemplated. Any gene involved (e.g., directly, indirectly) in the sialic acid biosynthesis pathway can be utilized (see FIG. 1). As one example, certain embodiments include methods for increasing sialic acid production by providing a subject with a wild-type GNE-encoding nucleic acid sequence that is optionally operably linked to a regulatory element, such as a promoter and/or enhancer sequence (see U.S. Application No. 2011/027373; WO 2008/097623; and U.S. Application No. 2009/029811, which are incorporated by reference in their entireties). This gene replacement therapy targets GNE/MNK, which is defective in HIBM patients, typically due to an autosomal recessive mutation of the GNE gene (see, e.g., Nemunaitis et al., The Journal of Gene Medicine 12:403-12, 2010). The GNE gene encodes the bi-functional enzyme UDP-GlcNAc 2-epimerase/ManNAc kinase.

Various viral vectors that can be utilized for gene replacement therapy include adenovirus, herpes virus, vaccinia, adeno-associated virus (AAV), or, preferably, an RNA virus such as a retrovirus. Preferably, the retroviral vector is a derivative of a murine or avian retrovirus, or is a lentiviral vector. The preferred retroviral vector is a lentiviral vector. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), SIV, BIV, HIV and Rous Sarcoma Virus (RSV). A number of additional retroviral vectors can incorporate multiple genes. All of these vectors can transfer or incorporate a gene for a selectable marker so that transduced cells can be identified and generated.

“Non-viral” delivery techniques for gene therapy can also be used including, for example, DNA-ligand complexes, adenovirus-ligand-DNA complexes, direct injection of DNA, CaPO₄ precipitation, gene gun techniques, electroporation, liposomes, lipofection, and the like. Any of these methods are widely available to one skilled in the art and would be suitable for use in the present invention. Other suitable methods are available to one skilled in the art, and it is to be understood that the present invention can be accomplished using any of the available methods of transfection. Lipofection can be accomplished by encapsulating an isolated DNA molecule within a liposomal particle and contacting the liposomal particle with the cell membrane of the target cell. Liposomes are self-assembling, colloidal particles in which a lipid bilayer, composed of amphiphilic molecules such as phosphatidyl serine or phosphatidyl choline, encapsulates a portion of the surrounding media such that the lipid bilayer surrounds a hydrophilic interior. Unilammellar or multilammellar liposomes can be constructed such that the interior contains a desired DNA molecule.

A desirable clinical or non-clinical outcome of the treatment(s) described herein includes, but is not limited to, increased production of sialic acid, restored level of sialylation in muscle and other proteins, increased muscle function, increased muscle strength (e.g., muscle strength of the quadriceps), increased muscle tensile force, improved muscle movement, improved limb movement, muscle growth, increased muscle stamina, decrease in muscle fatigability, decrease in muscle atrophy, decrease in neuronal atrophy, increase in pulmonary function, reduction in proteinuria (e.g., lower amounts of protein in the urine), reduction in hematuria (e.g., lower amounts of red blood cells in the urine) increased activity, stable disease (e.g., preventing or delaying the worsening of the disease), and/or increase or elongation of overall survival. The clinical outcome(s) will then be considered, and a decision as to whether the patient is suitable for the therapy will be made accordingly, taking into account the patient's specific situation and the relevance of the clinical or non-clinical outcomes.

Various pharmaceutical formulations comprising one or more therapeutic agents may be used in any of the methods described herein. In particular, provided herein are pharmaceutical formulations comprising one or more therapeutic agents (e.g., those described herein) and a pharmaceutically acceptable carrier, diluent, and/or excipient. Examples of suitable carriers, excipients, and diluents include, but are not limited to, sugars, lactose, dextrose, sucrose, sorbitol, mannitol, starches, gum such as xanthan gum, guar gum, or gum acacia, calcium phosphate, alginates, tragacanth, gelatin, calcium silicate, microcrystalline cellulose, polyethylene glycols, polyvinylpyrrolidone, phospholipics, cellulose, water, saline solution, syrup, methylcellulose, methyl- and propylhydroxybenzoates, talc, magnesium stearate, mineral oil, lubricating agents, wetting agents, emulsifying and suspending agents, preserving agents, sweetening agents, disintegrating agents, antioxidants, surfactants, and/or flavoring agents.

Pharmaceutical formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

The pharmaceutical formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use.

In some embodiments, when the therapeutic agent is a nucleic acid, the therapeutic agent may be used and delivered to a system in connection with an appropriate delivery vehicle (such as a liposome or lipid nanoparticle). In specific aspects, the nucleic acid is administered in conjunction with a lipid nanoparticle. Particular embodiments include a human non-viral GNE-plasmid embedded in cationic liposomes (e.g., GNE Lipoplex). Merely for illustrative purposes, these and other embodiments can be administered via intramuscular injection (e.g., biceps and extensor carpi radialis longus), intravenously (IV), or via intrahepatic (the major organ of SA synthesis) injections.

For topical administration, the pharmaceutical formulation may be a cream, milk, gel, dispersion, or microemulsions, lotion thickened to a greater or lesser extent, impregnated pad, ointment or stick, aerosol formulations (e.g., sprays or foams), soaps, detergents, lotions or cakes of soap.

The pharmaceutical formulation may be a food supplement or incorporated into food or drink item such as a nutritional bar, snack bar, cookie, candy, cereal, pudding, ice cream, frozen confectionary, chewing gum, drink mix, soda pop, liquid supplement, sauce, salad dressing, gravy, jelly, jam, spread, margarine, peanut butter, nut spread, frosting, and the like. In essence, can be used in any food, composition or supplement in which sugar is employed. Hence, the therapeutic agent and/or derivatives thereof can be used as a partial or full substitute for sugar.

Such food supplements, drinks and food items can include any other food ingredient including, for example, flour, oil, cream, butter, sugar, salt, spices and the like. In addition, the food supplements, drinks and food items can include vitamins and nutrients commonly found in other nutritional supplements.

Various routes of administration may be used in any of the methods described herein. In some embodiments of any of the methods described herein, the therapeutic agent can be administered by a variety of routes including oral, parenteral (including subcutaneous, intravenous, intramuscular, intraperitoneal, intraarticular, intraarterial, intrasynovial, or infusion techniques), rectal, dermal, transdermal, intrathoracic, intrapulmonary and intranasal (respiratory) routes.

Administration of the therapeutic agents in accordance may be in a single dose, in multiple doses, in a continuous or intermittent manner, depending, for example, upon the recipient's physiological condition, whether the purpose of the administration is therapeutic or prophylactic, and other factors known to skilled practitioners. The administration of the therapeutic agent may be essentially continuous over a pre-selected period of time or may be in a series of spaced doses. Both local and systemic administration is contemplated.

In certain of the methods described herein, the therapeutic agent is formulated for various forms of administration by any of the methods well known to the pharmaceutical arts. See, e.g., WO 2008/150477 and US 20090298112, incorporated herein in their entireties. The therapeutic agent may be administered, for example, at a dose of at least about 0.01 mg/kg to about 500 to 750 mg/kg, of at least about 0.01 mg/kg to about 300 to 500 mg/kg, at least about 0.1 mg/kg to about 200 to 400 mg/kg, at least about 1 mg/kg to about 25 mg/kg, or at least about 5 mg/kg to about 40 mg/kg, or at least about 1 mg/kg to 200 mg/kg, at least about 1 mg/kg to about 1000 mg/kg, at least about 200 mg/kg to about 1000 mg/kg, at least about 400 mg/kg to about 1000 mg/kg, or at least about 600 mg/kg to about 1000 mg/kg of body weight, although other dosages may provide beneficial results. The amount administered will vary depending on various factors including, but not limited to the disease, the weight, the physical condition, the health, the age of the mammal, whether prevention or treatment is to be achieved. Such factors can be readily determined by the clinician employing animal models or other test systems that are available in the art.

In some embodiments, the methods of the present invention include determining that the subject is suitable for sialic acid deficiency treatment based upon an increase or decrease in one or more SAT biomarkers. Optionally, in some embodiments, the increase or decrease in one or more biomarkers has been maintained for at least about 1, 2, 3, 4, 5, 6, 7, 8, 9, 10, 11, 12, 13, 14, 15, 16, 17, 18, 19, 20, 21, 22, 23, 24 weeks or at least about 1 month, 2 months, 3 months or 6 months or more prior to said determination.

In some embodiments the methods of the present invention include maintaining, increasing or reducing the dosage amount and/or frequency of the treatment upon an increase or decrease in one or more SAT biomarkers. Optionally, in some embodiments the increase or decrease in one or more SAT biomarkers has been maintained for at least about 1, 2, 3, 4, 5, 6, 7 days or at least about 1, 2, 3, 4, 5, 6, 7, 8, 9, 10, 11, 12, 13, 14, 15, 16, 17, 18, 19, 20, 21, 22, 23, 24 weeks or at least about 1 month, 2 months, 3 months or 6 months or more prior to maintaining, increasing or reducing the dosage amount and/or frequency of the treatment. In some embodiments, the dosage of a drug for treating sialic acid deficiencies can be tested, determined, or modified in view of the patient's response to the drug reflected by the level of one or more biomarkers of the present invention. In some embodiments, the dosage can be increased if the level of the biomarkers indicates that the subject is more responsive to a higher dosage of the drug. In some embodiments, the dosage can be decreased if the biomarkers indicates that the subject is more sensitive to the drug compared to the average subjects.

The dosage amount can be increased, merely by way of example, by about 1.1×, 1.2×, 1.3×, 1.4×, 1.5×, 1.6×, 1.7×, 1.8×, 1.9×, 2×, 2.5×, 3×, 3.5×, 4×, 4.5×, 5×, 6×, 7×, 8×, 9×, 10×, 15×, 20× or more, relative to the previous dosage. The dosage frequency can be increased, merely by way of illustration, by about 1, 2, 3, 4, 5 or more dosages per day, and/or 1, 2, 3, 4, 5, 6, 7, 8, 9, 10 or more dosages per week, relative to the previous dosing schedule. As noted above, the dosage amount can be increased separately or in combination with the dosage frequency, and vice versa, optionally until a desired level or range of one or more biomarkers or other treatment indicators is achieved. In some embodiments, the drug is administered to a subject at about 1 g/day, 2 g/day, 3 g/day, 4 g/day, 5 g/day, 6 g/day, 7 g/day, 8 g/day, 9 g/day, 10 g/day, or more.

Provided herein are kits and/or articles of manufacture comprising packaging material and at least one vial comprising an agent with the prescribed buffers and/or preservatives, optionally in an aqueous diluent, wherein the agent is used to determine and/or detect the level of one or more SAT biomarkers in a biological sample. Also provided herein are kits and/or articles of manufacture comprising packaging material and at least one vial comprising a therapeutic agent with the prescribed buffers and/or preservatives, optionally in an aqueous diluent. Further provided herein are kits and/or articles of manufacture comprising packaging material and at least one vial comprising an agent with the prescribed buffers and/or preservatives, optionally in an aqueous diluent, wherein the agent is used to the level of one or more SAT biomarkers in a biological sample and at least one vial comprising a therapeutic agent with the prescribed buffers and/or preservatives, optionally in an aqueous diluent.

Further provided herein are kits and/or articles of manufacture for use in treating an individual suffering from a sialic acid deficiency, identifying an individual as suitable or not suitable for treatment, and/or selecting an individual for treatment based on the level of one or more SAT biomarkers in a biological sample from the individual. The kit and/or article of manufacture comprises, or alternatively consists essentially of, or yet further consists of, one or more suitable agent(s) to determine the level of one or more SAT biomarkers, one or more therapeutic agent(s) and instructions for use thereof. In some embodiments, the kit and/or article of manufacture comprises, or alternatively consists essentially of, or yet further consists of, one or more suitable antibodies or probes, one or more therapeutic agent(s) and instructions for use thereof.

Provided herein are also kits and/or articles of manufacture for use in monitoring responsiveness or lack of responsiveness to treatment in an individual and/or identifying an individual as suitable or not suitable to continue treatment with a therapeutic agent based on level of one or more SAT biomarkers in a biological sample from the individual. The kit and/or article of manufacture comprises, or alternatively consists essentially of, or yet further consists of, one or more suitable agent to determine level of one or more SAT biomarkers, one or more therapeutic agent and instructions for use thereof. In some embodiments, the kit and/or article of manufacture comprises, or alternatively consists essentially of, or yet further consists of, one or more suitable antibody or probe, one or more therapeutic agent and instructions for use thereof. In some embodiments, the level of one or more SAT biomarkers indicates that the individual is non-responsive to current treatment, and might need optimization of treatment. In some embodiments, the level of one or more SAT biomarkers is compared between biological samples obtained before and after treatment and provides an indication that the individual is responsive or non-responsive to treatment. In some embodiments, the level of one or more SAT biomarkers is compared between biological samples obtained before or after treatment and a predetermined standard level and provides an indication that the individual is responsive or non-responsive to treatment.

In some embodiments, the biological sample is a blood sample (e.g., serum sample). In some embodiments, the biomarker is a SAT biomarker. In some embodiments, the sialic acid deficiency is Hereditary Inclusion Body Myopathy (HIBM).

The kits and/or articles of manufacture can include all or some of the positive controls, negative controls, reagents, antibodies and probes described herein for determining the level of one or more SAT biomarkers in a biological sample.

As amenable, these suggested kit and/or article of manufacture components may be packaged in a manner customary for use by those of skill in the art. For example, these suggested kit and/or article of manufacture components may be provided in solution or as a liquid dispersion or the like.

Included within the scope of the invention are DNA arrays or microarrays containing a plurality of sequences that hybridize under stringent hybridization conditions to one or more of the gene sequences of the biomarkers. An example of a substrate containing one or more probes of interest is a plurality of DNA probes that are affixed to a substrate. In certain embodiments, the substrate may comprise one or more materials such as gel, nitrocellulose, nylon, quartz, glass, metal, silica based materials, silica, resins, polymers, etc., or combinations thereof. Typically, the DNA probes comprise about 10-50 bp of contiguous DNA. In certain embodiments, the DNA probes are from about 20 to about 50 bp of contiguous DNA. In certain embodiments, the present invention relates to kits which comprising a microarray directions for its use. The kit may comprise a container which comprises one or more microarrays and directions for their use.

The biological sample may also be analyzed for gene expression of one or more gene markers using methods that can detect nucleic acids including, but not limited to, PCR (polymerase chain reaction); RT-PCT (reverse transcriptase-polymerase chain reaction); quantitative or semi-quantitative PCR, etc.

In certain embodiments, the levels of gene expression are measured by detecting the protein expression products of the genes or DNA sequences. The levels of protein products may be measured using methods known in the art including the use of antibodies which specifically bind to a particular protein. These antibodies, including polyclonal or monoclonal antibodies, may be produced using methods that are known in the art. These antibodies may also be coupled to a solid substrate to form an antibody chip or antibody microarray. Antibody or protein microarrays may be made using methods that are known in the art.

In some embodiments, the methods of the present invention can be applied on a dosage basis. For example, for each pre-determined dosage of the same drug, one or more biomarkers of the present invention can be used to determine if a specific subject is responding to a specific drug at the pre-determined dosage.

In some embodiments, the methods of the present invention can be applied on an administration method basis. For example, for each pre-determined drug administration method of the same drug, one or more biomarkers of the present invention can be used to determine if a specific subject is responding to the multi-kinase inhibitor by using the pre-determined drug administration method. None limiting examples of a route for administration include, mucosal, enteral, parental, transdermal/transmucosal, and inhalation. In one embodiment, the mucosal route is via the nasal, oropharyngeal, ocular, or genitourinary mucosa. In another embodiment, the enteral route is oral, rectal or sublingual. Still in another embodiment, the parenteral route is any one of intraarterial, intradermal, intramuscular, intraperitoneal, intravenous, subcutaneous, and submucosal injection or infusion. Still in another embodiment, the transdermal/transmucosal route is topical. Still in another embodiment, the inhalation route is intranasal, oropharyngeal, intratracheal, intrapulmonary or transpulmonary.

In some embodiments, the methods of the present invention can be applied on a drug combination basis. For example, for each pre-determined drug combination of a drug for treating sialic acid deficiencies, and a drug for treating other diseases, or a combination of two or more drugs for treating sialic acid deficiencies, one or more biomarkers of the present invention can be used to determine if a specific subject is responding to the drug combination.

In some embodiments, the methods of the present invention can be applied on a formulation basis. For example, for each pre-determined drug formulation of a given drug, one or more biomarkers of the present invention can be used to determine if a specific subject is responding to the multi-kinase inhibitor by using the pre-determined drug formulation.

The biomarkers and associated methods of the present invention can be used for all suitable purposes. In some embodiments, they are used in prospective clinical trial. In some embodiments, they are used in clinical treatment/prevention practice.

All publications, patent applications, and issued patents cited in this specification are herein incorporated by reference as if each individual publication, patent application, or issued patent were specifically and individually indicated to be incorporated by reference in its entirety.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. The following examples are provided by way of illustration only and not by way of limitation. Those of skill in the art will readily recognize a variety of non-critical parameters that could be changed or modified to yield essentially similar results.

### EXAMPLES

### Example 1

### Phase 1 Clinical Trial

### Use of Myriad RBM Human Discovery MAP250+ v1.0 to Identify Biomarkers that May be Involved in Heredity Inclusion Body Myopathy and Change During Treatment with Sialic Acid

HIBM is a genetic disorder characterized by progressive muscle weakness and wasting that develops in young adults. Muscle wasting usually starts around the age of 20-30 years, although young onset at 17 and old onset at 52 has been recorded. It can progress to marked disability within 10-15 years, confining many patients to the wheelchair. The weakness and severity can vary from person to person. In some, weakness in the legs is noticed first. In few others, the hands are weakened more rapidly than the legs. Weakness is progressive, which means the muscle become weaker over time. The quadriceps are relatively spared, and remain strong until the late stages of disease.

The current status of biomarker discovery for HIBM or other muscle diseases remains at early stage. One of the challenges is to identify useful surrogate markers in serum that can accurately reflect the changes in muscle and predict the effect of treatments on the disease.

Prior to this study, there had been limited biomarker data available for HIBM patients. No other studies have been done with the Human Discovery MAP250+ v1.0 to assess HIBM and to evaluate the efficacy of treatment in the disease. The present invention represents a major step toward identifying clinically relevant serum biomarkers that can be developed and used in clinical trials.

This study related to the search for serum biomarkers that are relevant to HIBM. The Human Discovery MAP250+ v1.0 quantitative immunoassay system from Myriad RBM was used (commercially available from Myriad). The system contains more than 250 serum markers (analytes) that cover dozens of biochemical pathways.

The immunoassay system is a product of Myriad RBM. The Human Discovery MAP250+ v1.0 platform consists of an extensive list of protein markers that are designed to be used in drug discovery and diagnostic development. The system is a multiplexed immunoassay and requires only a small sample volume.

Results of the 258 analytes (proteins markers) for the 18 HIBM patient serum samples were compiled. The mean, median and standard deviation were calculated. Data from 126 “clinically normal” individuals were used for comparison (see FIG. 1 of U.S. Provisional Application Ser. No. 61/779,929 filed on Mar. 13, 2013, and Table 2 below) in order to demonstrate statistical difference using Wilcoxon Rank Sum test. In addition, the ratios of median values between HIMB and normal samples were listed.

TABLE 2
Level of potential biomarkers in clinically normal individuals

| | | RBM | RBM | | | | | | |
|---|---|---|---|---|---|---|---|---|---|
| | | Low Serum | High Serum | | | | | | |
| | | Reference | Reference | Normal | Normal | Normal | | | Positive |
| 258 Analytes | Units | Range | Range | Mean | SD | Median | Min | Max | Count |
| 6Ckine | pg/mL | 250 | 895 | 536 | 182 | 520 | 201 | 1200 | 126 |
| Adiponectin | ug/mL | 1.1 | 11 | 4.4 | 2.6 | 3.8 | 1 | 14 | 126 |
| Agouti-Related | pg/mL | 54 | 317 | 196 | 86 | 183 | 54 | 719 | 126 |
| Protein (AGRP) | | | | | | | | | |
| Aldose Reductase | ng/mL | 0.72 | 6.5 | 3.4 | 1.7 | 3.4 | 0 | 12 | 125 |
| Alpha-1- | ug/ml | 465 | 2120 | 866 | 595 | 752 | 435 | 6400 | 126 |
| Antichymotrypsin | | | | | | | | | |
| (AACT) | | | | | | | | | |
| Alpha-1-Antitrypsin | mg/mL | 0.55 | 2.7 | 1.6 | 0.55 | 1.6 | 0 | 3 | 126 |
| (AAT) | | | | | | | | | |
| Alpha-1- | ug/ml | 6.7 | 18 | 11 | 2.9 | 11 | 6 | 23 | 126 |
| Microglobulin | | | | | | | | | |
| (A1Micro) | | | | | | | | | |
| Alpha-2- | mg/mL | 1.00 | 7.9 | 3.2 | 1.8 | 2.9 | 1 | 8 | 126 |
| Macroglobulin | | | | | | | | | |
| (A2Macro) | | | | | | | | | |
| Alpha-Fetoprotein | ng/mL | <LOW> | 15 | Clinical | Clinical | Clinical | Clinical | Clinical | Clinical |
| (AFP) | | | | Standard | Standard | Standard | Standard | Standard | Standard |
| Amphiregulin | pg/mL | <LOW> | 733 | 505 | 225 | 484 | 200 | 1060 | 24 |
| (AR) | | | | | | | | | |
| Angiogenin | ng/mL | 379 | 1200 | 623 | 191 | 588 | 260 | 1600 | 126 |
| Angiopoietin-2 | ng/mL | 1.0 | 7.0 | 3.2 | 1.5 | 3.0 | 0 | 9 | 126 |
| (ANG-2) | | | | | | | | | |
| Angiotensin- | ng/ml | 50 | 157 | 92 | 30 | 87 | 43 | 197 | 126 |
| Converting | | | | | | | | | |
| Enzyme (ACE) | | | | | | | | | |
| Angiotensinogen | ng/mL | 35 | 1340 | 555 | 366 | 493 | 30 | 1440 | 126 |
| Annexin A1 | ng/mL | <LOW> | 296 | 163 | 64 | 161 | 44 | 323 | 86 |
| (ANXA1) | | | | | | | | | |
| Apolipoprotein A-I | mg/mL | 0.75 | 2.1 | 1.3 | 0.36 | 1.3 | 1 | 3 | 126 |
| (Apo A-I) | | | | | | | | | |
| Apolipoprotein A-II | ng/ml | 228 | 1150 | 534 | 274 | 448 | 189 | 1560 | 126 |
| (Apo A-II) | | | | | | | | | |
| Apolipoprotein A-IV | ug/ml | 7.8 | 90 | 38 | 22 | 34 | 5 | 134 | 126 |
| (Apo A-IV) | | | | | | | | | |
| Apolipoprotein B | ug/ml | 1000 | 3550 | 2013 | 714 | 1830 | 835 | 5130 | 126 |
| (Apo B) | | | | | | | | | |
| Apolipoprotein C-I | ng/ml | 139 | 484 | 275 | 92 | 261 | 116 | 598 | 126 |
| (Apo C-I) | | | | | | | | | |
| Apolipoprotein C-III | ug/mL | 56 | 529 | 163 | 106 | 135 | 52 | 758 | 126 |
| (Apo C-III) | | | | | | | | | |
| Apolipoprotein D | ug/ml | 77 | 348 | 192 | 71 | 178 | 52 | 481 | 126 |
| (Apo D) | | | | | | | | | |
| Apolipoprotein E | ug/ml | 6.5 | 109 | 45 | 29 | 38 | 3 | 165 | 126 |
| (Apo E) | | | | | | | | | |
| Apolipoprotein H | ug/mL | 176 | 524 | 296 | 87 | 282 | 143 | 589 | 126 |
| (Apo H) | | | | | | | | | |
| Apolipoprotein(a) | ug/mL | 23 | 3310 | 781 | 875 | 506 | 1 | 3760 | 122 |
| (Lp(a)) | | | | | | | | | |
| AXL Receptor | ng/mL | 4.3 | 16 | 9.7 | 3.0 | 9.5 | 2 | 20 | 126 |
| Tyrosine Kinase | | | | | | | | | |
| (AXL) | | | | | | | | | |
| B cell-activating | pg/mL | 407 | 1150 | 684 | 191 | 654 | 259 | 1380 | 126 |
| factor (BAFF) | | | | | | | | | |
| B Lymphocyte | pg/ml | <LOW> | 60 | 19 | 42 | 8.6 | 6 | 245 | 67 |
| Chemoattractant | | | | | | | | | |
| (BLC) | | | | | | | | | |
| Bcl-2-like protein 2 | ng/mL | <LOW> | 2.5 | 1.7 | 2.5 | 1.1 | 1 | 16 | 34 |
| (Bcl2-L-2) | | | | | | | | | |
| Beta-2- | ug/mL | 1.0 | 2.8 | 1.7 | 0.47 | 1.6 | 1 | 3 | 126 |
| Microglobulin | | | | | | | | | |
| (B2M) | | | | | | | | | |
| Betacellulin (BTC) | pg/mL | <LOW> | 185 | 124 | 117 | 84 | 51 | 875 | 78 |
| Bone | ng/mL | <LOW> | 0.61 | 0.27 | 0.15 | 0.17 | 0 | 1 | 68 |
| Morphogenetic | | | | | | | | | |
| Protein 6 (BMP-6) | | | | | | | | | |
| Brain-Derived | ng/mL | 0.13 | 2.1 | 0.57 | 0.69 | 040 | 0 | 7 | 126 |
| Neurotrophic | | | | | | | | | |
| Factor(BDNF) | | | | | | | | | |
| Calbindin | ng/ml | <LOW> | 2.4 | 2.6 | 0.42 | 2.4 | 2 | 3 | 7 |
| Calcitonin | pg/mL | <LOW> | 17 | 8.3 | 5.0 | 6.6 | 3 | 20 | 67 |
| Cancer Antigen 125 | U/mL | <LOW> | 35 | Clinical | Clinical | Clinical | Clinical | Clinical | Clinical |
| (CA-125) | | | | Standard | Standard | Standard | Standard | Standard | Standard |
| Cancer Antigen 15- | U/mL | 1.2 | 9.6 | 4.5 | 2.2 | 4.3 | 1 | 12 | 126 |
| 3 (CA-15-3) | | | | | | | | | |
| Cancer Antigen 19- | U/mL | <LOW> | 52 | Clinical | Clinical | Clinical | Clinical | Clinical | Clinical |
| 9 (CA-19-9) | | | | Standard | Standard | Standard | Standard | Standard | Standard |
| Cancer Antigen 72- | U/mL | <LOW> | 21 | 19 | 52 | 3.7 | 2 | 279 | 30 |
| 4(CA-72-4) | | | | | | | | | |
| Carcinoembryonic | ng/mL | 0.40 | 5.6 | 2.2 | 2.6 | 1.6 | 0 | 28 | 124 |
| Antigen (CEA) | | | | | | | | | |
| Cathepsin D | ng/mL | 230 | 590 | 357 | 89 | 346 | 208 | 814 | 126 |
| CD 40 antigen | ng/mL | 0.26 | 0.68 | 0.44 | 0.11 | 0.42 | 0 | 1 | 126 |
| (CD40) | | | | | | | | | |
| CD40 Ligand | ng/mL | <LOW> | 0.055 | 0.031 | 0.064 | 0.019 | 0 | 0 | 54 |
| (CD40-L) | | | | | | | | | |
| CD5 (CD5L) | ng/ml | 771 | 3450 | 1624 | 647 | 1450 | 581 | 4580 | 126 |
| Cellular | ug/mL | <LOW> | 1.1 | 0.66 | 0.39 | 0.63 | 0 | 2 | 27 |
| Fibronectin (cFib) | | | | | | | | | |
| Chemokine CC-4 | ng/mL | 0.76 | 14 | 3.8 | 3.6 | 2.7 | 0 | 27 | 126 |
| (HCC-4) | | | | | | | | | |
| Chromogranin-A | ng/mL | <LOW> | 321 | 79 | 113 | 13 | 5 | 372 | 52 |
| (CgA) | | | | | | | | | |
| Ciliary | pg/mL | <LOW> | 12 | 14 | 2.6 | 13 | 12 | 17 | 4 |
| Neurotrophic Factor | | | | | | | | | |
| (CNTF) | | | | | | | | | |
| Clusterin (CLU) | ug/ml | 190 | 526 | 302 | 84 | 286 | 112 | 577 | 126 |
| Collagen IV | ng/mL | 45 | 198 | 93 | 43 | 78 | 26 | 331 | 126 |
| Complement C3 | mg/mL | 1.2 | 3.3 | 2.2 | 0.58 | 2.1 | 1 | 4 | 126 |
| (C3) | | | | | | | | | |
| Complement Factor | ug/ml | 900 | 5670 | 3333 | 1375 | 3330 | 704 | 6810 | 126 |
| H | | | | | | | | | |
| Connective Tissue | ng/ml | <LOW> | 1.9 | 1.1 | 0.57 | 0.71 | 0 | 3 | 29 |
| Growth Factor | | | | | | | | | |
| (CTGF) | | | | | | | | | |
| Cortisol (Cortisol) | ng/ml | 10 | 221 | 101 | 52 | 94 | 10 | 250 | 123 |
| C-peptide | ng/ml | 0.22 | 5.5 | 1.4 | 1.5 | 1.1 | 0 | 12 | 126 |
| C-Reactive Protein | ug/mL | <LOW> | 8.0 | Clinical | Clinical | Clinical | Clinical | Clinical | Clinical |
| (CRP) | | | | Standard | Standard | Standard | Standard | Standard | Standard |
| Creatine Kinase- | ng/mL | 0.12 | 3.5 | 1.1 | 0.93 | 0.80 | 0 | 6 | 125 |
| MB (CK-MB) | | | | | | | | | |
| Cystatin-C | ng/ml | 550 | 1720 | 1035 | 286 | 980 | 330 | 1780 | 126 |
| Endoglin | ng/mL | 2.2 | 6.2 | 3.9 | 1.0 | 3.8 | 1 | 7 | 126 |
| Endostatin | ng/mL | 52 | 122 | 79 | 19 | 77 | 33 | 144 | 126 |
| Endothelin-1 (ET-1) | pg/mL | <LOW> | 25 | 18 | 3.9 | 17 | 13 | 35 | 53 |
| EN-RAGE | ng/mL | 3.5 | 43 | 15 | 11 | 11 | 3 | 67 | 126 |
| Eotaxin-1 | pg/mL | <LOW> | 100 | 42 | 30 | 28 | 11 | 202 | 86 |
| Eotaxin-2 | pg/mL | 190 | 2670 | 839 | 626 | 653 | 86 | 3040 | 126 |
| Eotaxin-3 | pg/mL | <LOW> | 137 | 266 | 262 | 150 | 137 | 796 | 6 |
| Epidermal Growth | pg/mL | <LOW> | 41 | 12 | 44 | 4.7 | 2 | 432 | 102 |
| Factor (EGF) | | | | | | | | | |
| Epidermal Growth | ng/mL | 3.4 | 5.9 | 4.4 | 0.62 | 4.4 | 3 | 6 | 126 |
| Factor Receptor | | | | | | | | | |
| (EGFR) | | | | | | | | | |
| Epiregulin (EPR) | pg/mL | <LOW> | 44 | <LOW> | <LOW> | <LOW> | 44 | 44 | 1 |
| Epithelial cell | pg/mL | <LOW> | 155 | 109 | 34 | 105 | 67 | 197 | 31 |
| adhesion molecule | | | | | | | | | |
| (EpCam) | | | | | | | | | |
| Epithelial-Derived | ng/mL | 0.095 | 0.90 | 0.38 | 0.39 | 0.29 | 0 | 4 | 126 |
| Neutrophil- | | | | | | | | | |
| Activating Protein | | | | | | | | | |
| 78 (ENA-78) | | | | | | | | | |
| Erythropoietin | pg/mL | <LOW> | 33 | 14 | 8.4 | 15 | 2 | 41 | 95 |
| (EPO) | | | | | | | | | |
| E-Selectin | ng/mL | 6.1 | 34 | 19 | 7.7 | 19 | 1 | 47 | 126 |
| Ezrin | ng/mL | <LOW> | 4.4 | 5.1 | 1.0 | 5.1 | 4 | 6 | 2 |
| Factor VII | ng/mL | 66 | 633 | 310 | 167 | 304 | 54 | 855 | 126 |
| Fas Ligand (FasL) | pg/mL | <LOW> | 121 | 54 | 29 | 47 | 9 | 146 | 122 |
| FASLG Receptor | ng/mL | 2.3 | 24 | 10 | 12 | 8.0 | 2 | 134 | 123 |
| (FAS) | | | | | | | | | |
| Fatty Acid-Binding | ng/mL | 2.9 | 39 | 14 | 11 | 12 | 2 | 80 | 126 |
| Protein, adipocyte | | | | | | | | | |
| (FABP, adipocyte) | | | | | | | | | |
| Fatty Acid-Binding | ng/mL | <LOW> | 8.3 | 2.1 | 2.5 | 1.5 | 0 | 13 | 74 |
| Protein, heart | | | | | | | | | |
| (FABP, heart) | | | | | | | | | |
| Fatty Acid-Binding | ng/mL | 7.9 | 116 | 37 | 28 | 31 | 5 | 193 | 126 |
| Protein, liver | | | | | | | | | |
| (FABP, liver) | | | | | | | | | |
| Ferritin (FRTN) | ng/ml | 15 | 792 | Clinical | Clinical | Clinical | Clinical | Clinical | Clinical |
| | | | | Standard | Standard | Standard | Standard | Standard | Standard |
| Fetuin-A | ug/ml | 753 | 2170 | 1269 | 363 | 1200 | 697 | 2490 | 126 |
| Fibrinogen | mg/mL | <LOW> | 0.046 | 0.028 | 0.010 | 0.025 | 0 | 0 | 86 |
| Fibroblast Growth | pg/mL | 44 | 371 | 210 | 108 | 201 | 27 | 1030 | 125 |
| Factor 4 (FGF-4) | | | | | | | | | |
| Fibroblast Growth | pg/mL | <LOW> | 92 | 114 | 63 | 92 | 73 | 277 | 11 |
| Factor basic (FGF- | | | | | | | | | |
| basic) | | | | | | | | | |
| Fibulin-1C (Fib-1C) | ug/mL | 16 | 47 | 27 | 7.5 | 26 | 13 | 50 | 126 |
| Follicle-Stimulating | mIU/mL | 1.7 | 63 | 11 | 16 | 4.7 | 1 | 68 | 126 |
| Hormone (FSH) | | | | | | | | | |
| Galectin-3 | ng/mL | 3.4 | 19 | 11 | 4.1 | 10 | 3 | 27 | 125 |
| Gelsolin | ug/mL | 44 | 93 | 66 | 14 | 65 | 38 | 114 | 126 |
| Glucagon | pg/ml | <LOW> | <LOW> | <LOW> | <LOW> | <LOW> | 0 | 0 | 0 |
| Glucagon-like | pg/ml | <LOW> | 6.1 | 2.8 | 2.0 | 2.4 | 1 | 9 | 52 |
| Peptide 1, total | | | | | | | | | |
| (GLP-1 total) | | | | | | | | | |
| Glucose-6- | ng/mL | <LOW> | 24 | 20 | 20 | 15 | 4 | 83 | 17 |
| phosphate | | | | | | | | | |
| Isomerase (G6PI) | | | | | | | | | |
| Glutamate-Cysteine | ng/mL | <LOW> | 12 | 6.0 | 2.6 | 5.1 | 3 | 17 | 68 |
| Ligase Regulatory | | | | | | | | | |
| subunit (GCLR) | | | | | | | | | |
| Glutathione S- | ng/ml | 1.3 | 79 | 18 | 19 | 11 | 1 | 117 | 126 |
| Transferase alpha | | | | | | | | | |
| (GST-alpha) | | | | | | | | | |
| Glutathione S- | ng/mL | <LOW> | 13 | 4.7 | 3.2 | 4.2 | 1 | 14 | 82 |
| Transferase Mu 1 | | | | | | | | | |
| (GST-M1) | | | | | | | | | |
| Granulocyte | pg/mL | <LOW> | 19 | 8.6 | 5.6 | 7.3 | 3 | 48 | 122 |
| Colony- | | | | | | | | | |
| Stimulating Factor | | | | | | | | | |
| (G-CSF) | | | | | | | | | |
| Granulocyte- | pg/mL | <LOW> | 8.5 | 11 | 4.0 | 8.5 | 8 | 18 | 5 |
| Macrophage | | | | | | | | | |
| Colony- | | | | | | | | | |
| Stimulating Factor | | | | | | | | | |
| (GM-CSF) | | | | | | | | | |
| Growth Hormone | ng/mL | <LOW> | 4.6 | 1.0 | 2.0 | 0.35 | 0 | 17 | 103 |
| (GH) | | | | | | | | | |
| Haptoglobin | mg/mL | 0.21 | 5.4 | 1.9 | 1.4 | 1.5 | 0 | 7 | 126 |
| HE4 | pM | <LOW> | 78 | 38 | 18 | 37 | 16 | 98 | 106 |
| Heat Shock Protein | ng/ml | <LOW> | 29 | 15 | 33 | 6.7 | 3 | 213 | 41 |
| 60 (HSP-60) | | | | | | | | | |
| Heparin-Binding | pg/mL | <LOW> | 43 | 29 | 20 | 23 | 12 | 94 | 18 |
| EGF-Like Growth | | | | | | | | | |
| Factor (HB-EGF) | | | | | | | | | |
| Hepatocyte Growth | ng/mL | 0.31 | 8.0 | 3.1 | 2.1 | 2.6 | 0 | 11 | 123 |
| Factor (HGF) | | | | | | | | | |
| Hepatocyte | ng/mL | 40 | 87 | 63 | 12 | 64 | 36 | 109 | 126 |
| Growth Factor | | | | | | | | | |
| receptor (HGF | | | | | | | | | |
| receptor) | | | | | | | | | |
| Hepsin | pg/mL | 465 | 1250 | 815 | 187 | 820 | 227 | 1360 | 126 |
| Human Chorionic | mIU/mL | <LOW> | 5.5 | 2.3 | 5.9 | 1.4 | 0 | 56 | 92 |
| Gonadotropin beta | | | | | | | | | |
| (hCG) | | | | | | | | | |
| Human Epidermal | ng/mL | 0.33 | 0.92 | 0.59 | 0.17 | 0.57 | 0 | 1 | 126 |
| Growth Factor | | | | | | | | | |
| Receptor 2 (HER-2) | | | | | | | | | |
| Immunoglobulin A | mg/mL | 1.5 | 8.0 | 3.8 | 1.7 | 3.4 | 1 | 10 | 126 |
| (IgA) | | | | | | | | | |
| Immunoglobulin E | IU/mL | <LOW> | 180 | Clinical | Clinical | Clinical | Clinical | Clinical | Clinical |
| (IgE) | | | | Standard | Standard | Standard | Standard | Standard | Standard |
| Immunoglobulin M | mg/mL | 0.79 | 6.6 | 2.9 | 1.4 | 2.6 | 1 | 8 | 126 |
| (IGM) | | | | | | | | | |
| Insulin | uIU/mL | 0.34 | 32 | 5.4 | 11 | 2.3 | 0 | 98 | 124 |
| Insulin-like | ng/mL | 258 | 816 | 520 | 153 | 511 | 138 | 1000 | 126 |
| Growth Factor | | | | | | | | | |
| Binding Protein 4 | | | | | | | | | |
| (IGFBP4) | | | | | | | | | |
| Insulin-like Growth | ng/mL | 100 | 216 | 148 | 30 | 145 | 78 | 250 | 126 |
| Factor Binding | | | | | | | | | |
| Protein 5 (IGFBP5) | | | | | | | | | |
| Insulin-like | ng/mL | 13 | 326 | 126 | 82 | 105 | 9 | 420 | 126 |
| Growth Factor | | | | | | | | | |
| Binding Protein 6 | | | | | | | | | |
| (IGFBP6) | | | | | | | | | |
| Insulin-like Growth | ng/mL | <LOW> | 61 | 11 | 24 | 3.7 | 1 | 215 | 109 |
| Factor-Binding | | | | | | | | | |
| Protein 1 (IGFBP-1) | | | | | | | | | |
| Insulin-like | ng/mL | 21 | 193 | 80 | 49 | 70 | 16 | 278 | 126 |
| Growth Factor- | | | | | | | | | |
| Binding Protein 2 | | | | | | | | | |
| (IGFBP-2) | | | | | | | | | |
| Insulin-like | ng/mL | 983 | 3910 | 2768 | 738 | 2805 | 139 | 4370 | 126 |
| Growth Factor- | | | | | | | | | |
| Binding Protein 3 | | | | | | | | | |
| (IGFBP-3) | | | | | | | | | |
| Intercellular | ng/mL | <LOW> | 195 | 98 | 48 | 97 | 28 | 242 | 121 |
| Adhesion Molecule | | | | | | | | | |
| 1 (ICAM-1) | | | | | | | | | |
| Interferon gamma | pg/mL | <LOW> | 4.3 | 2.0 | 1.7 | 1.3 | 1 | 11 | 61 |
| (IFN-gamma) | | | | | | | | | |
| Interferon gamma | pg/ml | 107 | 721 | 313 | 244 | 272 | 52 | 2360 | 126 |
| Induced Protein 10 | | | | | | | | | |
| (IP-10) | | | | | | | | | |
| Interferon- | pg/mL | <LOW> | 43 | 15 | 10 | 11 | 5 | 57 | 108 |
| inducible T-cell | | | | | | | | | |
| alpha | | | | | | | | | |
| chemoattractant | | | | | | | | | |
| (ITAC) | | | | | | | | | |
| Interleukin-1 alpha | ng/mL | <LOW> | 0.0040 | 0.0021 | 0.0019 | 0.0018 | 0 | 0 | 85 |
| (IL-1 alpha) | | | | | | | | | |
| Interleukin-1 beta | pg/mL | <LOW> | 0.52 | 0.63 | 0.12 | 0.65 | 1 | 1 | 5 |
| (IL-1 beta) | | | | | | | | | |
| Interleukin-1 | pg/mL | <LOW> | 135 | 61 | 35 | 51 | 14 | 242 | 96 |
| receptor antagonist | | | | | | | | | |
| (IL-1ra) | | | | | | | | | |
| Interleukin-10 (1L- | pg/mL | <LOW> | 7.9 | 4.0 | 2.0 | 3.7 | 2 | 14 | 72 |
| 10) | | | | | | | | | |
| Interleukin-12 | ng/mL | <LOW> | 0.18 | 0.15 | 0.051 | 0.12 | 0 | 0 | 25 |
| Subunit p40 (IL- | | | | | | | | | |
| 12p40) | | | | | | | | | |
| Interleukin-12 | pg/mL | <LOW> | <LOW> | <LOW> | <LOW> | <LOW> | 0 | 0 | 0 |
| Subunit p70 (IL- | | | | | | | | | |
| 12p70) | | | | | | | | | |
| Interleukin-13 (IL- | pg/mL | 13 | 58 | 41 | 12 | 41 | 3 | 132 | 124 |
| 13) | | | | | | | | | |
| Interleukin-15 (IL- | ng/mL | <LOW> | 0.61 | 0.35 | 0.20 | 0.29 | 0 | 2 | 68 |
| 15) | | | | | | | | | |
| Interleukin-16 (IL- | pg/mL | 99 | 330 | 208 | 62 | 202 | 52 | 393 | 126 |
| 16) | | | | | | | | | |
| Interleukin-18 (IL- | pg/mL | 86 | 538 | 216 | 102 | 194 | 68 | 713 | 126 |
| 18) | | | | | | | | | |
| Interleukin-2 (IL-2) | pg/mL | <LOW> | 15 | 15 | 7.5 | 13 | 10 | 33 | 13 |
| Interleukin-2 | pg/mL | 637 | 2970 | 1444 | 556 | 1390 | 566 | 3880 | 126 |
| receptor alpha (IL- | | | | | | | | | |
| 2 receptor alpha) | | | | | | | | | |
| Interleukin-25 (IL- | pg/mL | <LOW> | 25 | 11 | 6.6 | 8.7 | 4 | 45 | 78 |
| 25) | | | | | | | | | |
| Interleukin-3 (IL- | ng/mL | <LOW> | 0.025 | 0.011 | 0.0089 | 0.0092 | 0 | 0 | 76 |
| 3) | | | | | | | | | |
| Interleukin-4 (IL- | pg/mL | <LOW> | 50 | 26 | 12 | 22 | 9 | 54 | 74 |
| 4) | | | | | | | | | |
| Interleukin-5 (IL- | pg/mL | <LOW> | 5.5 | 2.9 | 2.6 | 2.2 | 1 | 23 | 76 |
| 5) | | | | | | | | | |
| Interleukin-6 (IL-6) | pg/mL | <LOW> | 5.0 | 3.5 | 6.1 | 2.0 | 1 | 41 | 43 |
| Interleukin-6 | ng/mL | 9.5 | 39 | 23 | 7.4 | 21 | 5 | 41 | 126 |
| receptor (IL-6r) | | | | | | | | | |
| Interleukin-6 | ng/mL | 118 | 271 | 185 | 37 | 185 | 109 | 347 | 126 |
| receptor subunit | | | | | | | | | |
| beta(IL-6R beta) | | | | | | | | | |
| Interleukin-7 (IL- | pg/mL | <LOW> | 40 | 21 | 26 | 14 | 4 | 185 | 54 |
| 7) | | | | | | | | | |
| Interleukin-8 (IL- | pg/mL | 3.9 | 289 | 51 | 69 | 20 | 3 | 344 | 125 |
| 8) | | | | | | | | | |
| Kallikrein 5 | ng/mL | 0.17 | 2.0 | 0.96 | 0.51 | 0.89 | 0 | 3 | 126 |
| Kallikrein-7 (KLK-7) | pg/mL | <LOW> | 1170 | 626 | 312 | 531 | 287 | 1890 | 86 |
| Kidney Injury | ng/ml | 0.019 | 0.69 | 0.16 | 0.34 | 0.089 | 0 | 3 | 126 |
| Molecule-1 (KIM-1) | | | | | | | | | |
| Lactoylglutathione | ng/mL | 1.6 | 6.3 | 3.4 | 1.4 | 3.1 | 0 | 11 | 126 |
| lyase (LGL) | | | | | | | | | |
| Latency- | ng/mL | 0.49 | 2.6 | 1.1 | 0.55 | 0.99 | 0 | 5 | 126 |
| Associated Peptide | | | | | | | | | |
| of Transforming | | | | | | | | | |
| Growth Factor | | | | | | | | | |
| beta 1 (LAP TGF-b1) | | | | | | | | | |
| Lectin-Like | ng/mL | <LOW> | 0.62 | 0.60 | 0.13 | 0.53 | 0 | 1 | 15 |
| Oxidized LDL | | | | | | | | | |
| Receptor 1 (LOX-1) | | | | | | | | | |
| Leptin | ng/mL | 0.34 | 39 | 12 | 14 | 8.6 | 0 | 96 | 126 |
| Luteinizing | mIU/mL | 1.2 | 22 | 6.1 | 5.1 | 4.2 | 1 | 24 | 126 |
| Hormone (LH) | | | | | | | | | |
| Lymphotactin | ng/mL | <LOW> | <LOW> | 0.18 | 0.11 | 0.13 | 0 | 0 | 3 |
| Macrophage | ng/mL | 0.051 | 0.17 | 0.100 | 0.033 | 0.092 | 0 | 0 | 126 |
| Colony- | | | | | | | | | |
| Stimulating Factor | | | | | | | | | |
| 1 (M-CSF) | | | | | | | | | |
| Macrophage | pg/mL | 42 | 551 | 268 | 616 | 171 | 32 | 6760 | 126 |
| inflammatory | | | | | | | | | |
| protein 3 beta (MIP- | | | | | | | | | |
| 3 beta) | | | | | | | | | |
| Macrophage | pg/mL | 30 | 159 | 63 | 37 | 56 | 25 | 339 | 126 |
| Inflammatory | | | | | | | | | |
| Protein-1 alpha | | | | | | | | | |
| (MIP-1 alpha) | | | | | | | | | |
| Macrophage | pg/mL | 41 | 751 | 210 | 995 | 93 | 21 | 11100 | 126 |
| Inflammatory | | | | | | | | | |
| Protein-1 beta | | | | | | | | | |
| (MIP-1 beta) | | | | | | | | | |
| Macrophage | pg/ml | <LOW> | 176 | 87 | 88 | 73 | 14 | 680 | 106 |
| Inflammatory | | | | | | | | | |
| Protein-3 alpha | | | | | | | | | |
| (MIP-3 alpha) | | | | | | | | | |
| Macrophage | ng/mL | 0.0092 | 0.17 | 0.056 | 0.046 | 0.044 | 0 | 0 | 123 |
| Migration Inhibitory | | | | | | | | | |
| Factor (MIF) | | | | | | | | | |
| Macrophage- | pg/mL | 216 | 674 | 414 | 124 | 414 | 122 | 826 | 126 |
| Derived Chemokine | | | | | | | | | |
| (MDC) | | | | | | | | | |
| Macrophage- | ng/mL | 111 | 1100 | 506 | 264 | 474 | 82 | 1350 | 126 |
| Stimulating | | | | | | | | | |
| Protein (MSP) | | | | | | | | | |
| Malondialdehyde- | ng/mL | <LOW> | 289 | 228 | 98 | 148 | 148 | 446 | 23 |
| Modified Low- | | | | | | | | | |
| Density | | | | | | | | | |
| Lipoprotein | | | | | | | | | |
| (MDA-LDL) | | | | | | | | | |
| Maspin | pg/mL | <LOW> | 2590 | 1327 | 582 | 1160 | 863 | 3820 | 76 |
| Matrix | ng/ml | <LOW> | 2.2 | 1.2 | 0.97 | 0.95 | 0 | 10 | 117 |
| Metalloproteinase- | | | | | | | | | |
| 1 (MMP-1) | | | | | | | | | |
| Matrix | ng/ml | 0.15 | 2.6 | 0.71 | 0.66 | 0.55 | 0 | 4 | 126 |
| Metalloproteinase- | | | | | | | | | |
| 10 (MMP-10) | | | | | | | | | |
| Matrix | ng/mL | <LOW> | 24 | 14 | 6.4 | 13 | 5 | 43 | 73 |
| Metalloproteinase-2 | | | | | | | | | |
| (MMP-2) | | | | | | | | | |
| Matrix | ng/mL | 1.0 | 13 | 4.3 | 3.8 | 3.1 | 1 | 28 | 126 |
| Metalloproteinase- | | | | | | | | | |
| 3 (MMP-3) | | | | | | | | | |
| Matrix | ng/ml | 2.0 | 7.5 | 3.8 | 2.3 | 3.4 | 1 | 24 | 126 |
| Metalloproteinase-7 | | | | | | | | | |
| (MMP-7) | | | | | | | | | |
| Matrix | ng/mL | <LOW> | 13 | 7.9 | 4.8 | 4.5 | 5 | 19 | 13 |
| Metalloproteinase- | | | | | | | | | |
| 9 (MMP-9) | | | | | | | | | |
| Matrix | ng/ml | 49 | 202 | 119 | 54 | 114 | 42 | 569 | 126 |
| Metalloproteinase-9, | | | | | | | | | |
| total (MMP-9, total) | | | | | | | | | |
| Mesothelin | nM | 26 | 127 | 63 | 31 | 56 | 12 | 252 | 126 |
| (MSLN) | | | | | | | | | |
| MHC class I chain- | pg/mL | <LOW> | 52 | <LOW> | <LOW> | <LOW> | 52 | 52 | 1 |
| related protein 1 | | | | | | | | | |
| (MICA) | | | | | | | | | |
| Monocyte | pg/mL | 55 | 238 | 131 | 49 | 130 | 46 | 312 | 126 |
| Chemotactic | | | | | | | | | |
| Protein 1 (MCP-1) | | | | | | | | | |
| Monocyte | pg/ml | 17 | 109 | 33 | 24 | 28 | 9 | 234 | 126 |
| Chemotactic | | | | | | | | | |
| Protein 2 (MCP-2) | | | | | | | | | |
| Monocyte | pg/mL | <LOW> | 9.8 | 3.9 | 5.0 | 2.3 | 1 | 27 | 33 |
| Chemotactic Protein | | | | | | | | | |
| 3 (MCP-3) | | | | | | | | | |
| Monocyte | pg/ml | 294 | 1240 | 707 | 272 | 697 | 146 | 1660 | 126 |
| Chemotactic | | | | | | | | | |
| Protein 4 (MCP-4) | | | | | | | | | |
| Monokine Induced | pg/ml | 462 | 3020 | 1119 | 825 | 937 | 359 | 5730 | 126 |
| by Gamma | | | | | | | | | |
| Interferon (MIG) | | | | | | | | | |
| Myeloid | ng/mL | 0.33 | 1.8 | 0.80 | 0.50 | 0.67 | 0 | 4 | 126 |
| Progenitor | | | | | | | | | |
| Inhibitory Factor 1 | | | | | | | | | |
| (MPIF-1) | | | | | | | | | |
| Myeloperoxidase | ng/mL | 46 | 629 | 199 | 139 | 158 | 46 | 716 | 124 |
| (MPO) | | | | | | | | | |
| Myoglobin | ng/mL | 5.0 | 43 | 15 | 12 | 11 | 4 | 89 | 126 |
| Nerve Growth | ng/mL | <LOW> | 0.048 | 0.051 | 0.032 | 0.034 | 0 | 0 | 9 |
| Factor beta (NGF- | | | | | | | | | |
| beta) | | | | | | | | | |
| Neuron Specific | ng/mL | <LOW> | 43 | 14 | 11 | 10 | 2 | 84 | 112 |
| Enolase (NSE) | | | | | | | | | |
| Neuronal Cell | ng/mL | <LOW> | 3.4 | 1.6 | 0.89 | 1.5 | 0 | 6 | 119 |
| Adhesion Molecule | | | | | | | | | |
| (Nr-CAM) | | | | | | | | | |
| Neuropilin-1 | ng/mL | 107 | 245 | 167 | 36 | 161 | 103 | 319 | 126 |
| Neutrophil | ng/ml | <LOW> | 209 | 106 | 48 | 88 | 40 | 263 | 102 |
| Gelatinase- | | | | | | | | | |
| Associated | | | | | | | | | |
| Lipocalin (NGAL) | | | | | | | | | |
| N-terminal | pg/ml | <LOW> | 425 | 288 | 1245 | 56 | 29 | 10200 | 70 |
| prohormone of brain | | | | | | | | | |
| natriuretic peptide | | | | | | | | | |
| (NT proBNP) | | | | | | | | | |
| Nucleoside | ng/mL | <LOW> | 3.3 | 1.6 | 0.68 | 1.4 | 0 | 4 | 121 |
| diphosphate kinase | | | | | | | | | |
| B(NDK B) | | | | | | | | | |
| Osteopontin | ng/ml | 1.6 | 18 | 5.1 | 6.8 | 3.6 | 1 | 66 | 126 |
| Osteoprotegerin | pM | 2.6 | 7.6 | 4.6 | 1.4 | 4.2 | 2 | 8 | 126 |
| (OPG) | | | | | | | | | |
| Pancreatic | pg/ml | 12 | 349 | 104 | 102 | 70 | 8 | 493 | 126 |
| Polypeptide (PPP) | | | | | | | | | |
| Pepsinogen I (PGI) | ng/mL | 29 | 281 | 110 | 62 | 106 | 5 | 390 | 126 |
| Peptide YY (PYY) | pg/mL | 17 | 236 | 133 | 57 | 128 | 17 | 299 | 123 |
| Peroxiredoxin 4 | ng/mL | 0.14 | 1.1 | 0.51 | 0.26 | 0.47 | 0 | 2 | 126 |
| (Prx-IV) | | | | | | | | | |
| Phosphoserine | ng/mL | 0.81 | 8.1 | 2.9 | 3.7 | 2.1 | 1 | 39 | 126 |
| Aminotransferase | | | | | | | | | |
| (PSAT) | | | | | | | | | |
| Placenta Growth | pg/ml | <LOW> | 43 | 24 | 11 | 22 | 12 | 67 | 72 |
| Factor (PLGF) | | | | | | | | | |
| Plasminogen | ng/mL | 6.9 | 115 | 32 | 29 | 23 | 4 | 166 | 126 |
| Activator Inhibitor | | | | | | | | | |
| 1 (PAI-1) | | | | | | | | | |
| Platelet-Derived | pg/ml | <LOW> | 960 | 271 | 327 | 167 | 42 | 2390 | 79 |
| Growth Factor BB | | | | | | | | | |
| (PDGF-BB) | | | | | | | | | |
| Pregnancy- | mIU/mL | <LOW> | 0.018 | 0.0086 | 0.0048 | 0.0074 | 0 | 0 | 64 |
| Associated Plasma | | | | | | | | | |
| Protein A (PAPP-A) | | | | | | | | | |
| Progesterone | ng/ml | <LOW> | 15 | 6.0 | 4.2 | 4.8 | 1 | 30 | 119 |
| Proinsulin, Intact | pM | <LOW> | 10 | 5.8 | 3.6 | 5.5 | 2 | 22 | 43 |
| Proinsulin, Total | pM | <LOW> | 41 | 15 | 15 | 11 | 4 | 93 | 68 |
| Prolactin (PRL) | ng/ml | 1.4 | 43 | 7.6 | 11 | 4.7 | 1 | 80 | 126 |
| Prostasin | ng/mL | 89 | 421 | 206 | 95 | 182 | 74 | 804 | 126 |
| Prostate-Specific | ng/mL | <LOW> | 0.27 | 0.068 | 0.089 | 0.041 | 0 | 0 | 97 |
| Antigen, Free (PSA-f) | | | | | | | | | |
| Prostatic Acid | ng/mL | <LOW> | 0.89 | 0.46 | 0.22 | 0.44 | 0 | 1 | 114 |
| Phosphatase (PAP) | | | | | | | | | |
| Protein S100-A4 | ng/mL | <LOW> | 19 | 6.6 | 7.8 | 4.6 | 0 | 63 | 102 |
| (S100-A4) | | | | | | | | | |
| Protein S100-A6 | ng/mL | 1.9 | 20 | 6.9 | 5.1 | 6.0 | 2 | 40 | 124 |
| (S100-A6) | | | | | | | | | |
| Pulmonary and | ng/mL | 35 | 182 | 91 | 39 | 84 | 27 | 227 | 126 |
| Activation- | | | | | | | | | |
| Regulated | | | | | | | | | |
| Chemokine (PARC) | | | | | | | | | |
| Receptor for | ng/mL | 0.41 | 6.3 | 2.5 | 1.7 | 2.0 | 0 | 7 | 125 |
| advanced | | | | | | | | | |
| glycosylation end | | | | | | | | | |
| products (RAGE) | | | | | | | | | |
| Receptor tyrosine- | ng/mL | 0.25 | 3.6 | 2.2 | 0.85 | 2.3 | 0 | 4 | 126 |
| protein kinase | | | | | | | | | |
| erbB-3 (ErbB3) | | | | | | | | | |
| Resistin | ng/ml | 1.4 | 5.6 | 2.7 | 1.1 | 2.3 | 1 | 7 | 126 |
| S100 calcium- | ng/mL | <LOW> | 0.85 | 0.38 | 0.37 | 0.18 | 0 | 1 | 33 |
| binding protein B | | | | | | | | | |
| (S100-B) | | | | | | | | | |
| Secretin | ng/mL | <LOW> | 0.71 | 4.3 | 3.0 | 4.6 | 1 | 7 | 4 |
| Serotransferrin | mg/dl | 195 | 534 | 327 | 90 | 305 | 188 | 657 | 126 |
| (Transferrin) | | | | | | | | | |
| Serum Amyloid P- | ug/mL | 12 | 42 | 25 | 8.0 | 24 | 9 | 50 | 126 |
| Component (SAP) | | | | | | | | | |
| Serum Glutamic | ug/mL | 1.4 | 5.3 | 2.5 | 0.97 | 2.2 | 1 | 8 | 126 |
| Oxaloacetic | | | | | | | | | |
| Transaminase | | | | | | | | | |
| (SGOT) | | | | | | | | | |
| Sex Hormone- | nmol/L | 20 | 133 | 57 | 31 | 51 | 16 | 220 | 126 |
| Binding Globulin | | | | | | | | | |
| (SHBG) | | | | | | | | | |
| Sortilin | ng/mL | 2.8 | 8.6 | 5.1 | 1.5 | 4.9 | 1 | 9 | 126 |
| Squamous Cell | ng/mL | 0.079 | 1.8 | 0.50 | 0.65 | 0.36 | 0 | 6 | 125 |
| Carcinoma Antigen- | | | | | | | | | |
| 1 (SCCA-1) | | | | | | | | | |
| Stem Cell Factor | pg/mL | 105 | 421 | 212 | 92 | 199 | 24 | 879 | 126 |
| (SCF) | | | | | | | | | |
| Stromal cell- | pg/mL | 720 | 2620 | 1614 | 523 | 1670 | 516 | 3730 | 126 |
| derived factor-1 | | | | | | | | | |
| (SDF-1) | | | | | | | | | |
| Superoxide | ng/mL | 3.6 | 17 | 7.5 | 4.7 | 6.2 | 3 | 43 | 126 |
| Dismutase 1, | | | | | | | | | |
| Soluble (SOD-1) | | | | | | | | | |
| T Lymphocyte- | pg/mL | 33 | 2650 | 453 | 1713 | 116 | 22 | 17700 | 125 |
| Secreted Protein I- | | | | | | | | | |
| 309 (I-309) | | | | | | | | | |
| Tamm-Horsfall | ug/ml | 0.016 | 0.079 | 0.049 | 0.018 | 0.049 | 0 | 0 | 126 |
| Urinary | | | | | | | | | |
| Glycoprotein (THP) | | | | | | | | | |
| T-Cell-Specific | ng/mL | 0.31 | 5.2 | 1.4 | 1.7 | 1.1 | 0 | 14 | 126 |
| Protein RANTES | | | | | | | | | |
| (RANTES) | | | | | | | | | |
| Tenascin-C (TN-C) | ng/mL | 189 | 927 | 466 | 241 | 423 | 184 | 2160 | 126 |
| Testosterone, Total | ng/ml | 0.20 | 16 | Clinical | Clinical | Clinical | Clinical | Clinical | Clinical |
| | | | | Standard | Standard | Standard | Standard | Standard | Standard |
| Tetranectin | ug/mL | 9.4 | 20 | 14 | 2.8 | 14 | 8 | 21 | 126 |
| Thrombomodulin | ng/ml | 2.9 | 8.5 | 5.1 | 1.4 | 4.8 | 2 | 9 | 126 |
| (TM) | | | | | | | | | |
| Thrombopoietin | ng/mL | <LOW> | 2.8 | 1.6 | 0.62 | 1.5 | 0 | 3 | 72 |
| (TPO) | | | | | | | | | |
| Thrombospondin-1 | ng/mL | 85 | 3380 | 876 | 1160 | 584 | 44 | 8460 | 125 |
| Thyroglobulin | ng/mL | 3.9 | 223 | 57 | 99 | 34 | 2 | 993 | 126 |
| (TG) | | | | | | | | | |
| Thyroid-Stimulating | uIU/mL | 0.15 | 4.0 | 1.5 | 2.2 | 1.1 | 0 | 23 | 125 |
| Hormone (TSH) | | | | | | | | | |
| Thyroxine-Binding | ug/mL | 35 | 113 | 65 | 18 | 63 | 27 | 131 | 126 |
| Globulin (TBG) | | | | | | | | | |
| Tissue Factor (TF) | ng/mL | <LOW> | 2.1 | 1.8 | 0.81 | 1.9 | 0 | 3 | 8 |
| Tissue Inhibitor of | ng/mL | 42 | 114 | 71 | 19 | 70 | 35 | 136 | 126 |
| Metalloproteinases | | | | | | | | | |
| 1 (TIMP-1) | | | | | | | | | |
| Tissue type | ng/mL | 0.28 | 1.8 | 0.93 | 0.47 | 0.81 | 0 | 2 | 126 |
| Plasminogen | | | | | | | | | |
| activator (tPA) | | | | | | | | | |
| TNF-Related | ng/mL | 6.0 | 35 | 16 | 7.2 | 14 | 5 | 40 | 126 |
| Apoptosis-Inducing | | | | | | | | | |
| Ligand Receptor 3 | | | | | | | | | |
| (TRAIL-R3) | | | | | | | | | |
| Transforming | pg/mL | <LOW> | <LOW> | <LOW> | <LOW> | <LOW> | 0 | 0 | 0 |
| Growth Factor alpha | | | | | | | | | |
| (TGF-alpha) | | | | | | | | | |
| Transforming | pg/mL | <LOW> | 13 | 68 | 93 | 13 | 13 | 243 | 8 |
| Growth Factor beta- | | | | | | | | | |
| 3 (TGF-beta-3) | | | | | | | | | |
| Transthyretin (TTR) | mg/dl | 13 | 66 | 33 | 13 | 32 | 9 | 69 | 126 |
| Trefoil Factor 3 | ug/ml | 0.018 | 0.14 | 0.064 | 0.030 | 0.058 | 0 | 0 | 125 |
| (TFF3) | | | | | | | | | |
| Tumor Necrosis | pg/mL | <LOW> | 21 | 6.0 | 6.3 | 3.8 | 2 | 37 | 62 |
| Factor alpha | | | | | | | | | |
| (TNF-alpha) | | | | | | | | | |
| Tumor Necrosis | pg/mL | <LOW> | 22 | 12 | 8.9 | 10 | 6 | 61 | 52 |
| Factor beta (TNF- | | | | | | | | | |
| beta) | | | | | | | | | |
| Tumor Necrosis | ng/mL | 3.4 | 11 | 5.5 | 1.6 | 5.3 | 2 | 12 | 126 |
| Factor Receptor 2 | | | | | | | | | |
| (TNFR2) | | | | | | | | | |
| Tumor Necrosis | pg/mL | 692 | 2020 | 1246 | 394 | 1215 | 518 | 3190 | 126 |
| Factor Receptor I | | | | | | | | | |
| (TNF RI) | | | | | | | | | |
| Tyrosine kinase | ng/mL | 8.4 | 23 | 15 | 3.6 | 15 | 5 | 28 | 126 |
| with Ig and EGF | | | | | | | | | |
| homology domains | | | | | | | | | |
| 2 (TIE-2) | | | | | | | | | |
| Urokinase-type | pg/mL | 381 | 1450 | 827 | 290 | 822 | 45 | 1810 | 126 |
| Plasminogen | | | | | | | | | |
| Activator (uPA) | | | | | | | | | |
| Urokinase-type | ng/mL | 0.51 | 3.5 | 1.5 | 0.84 | 1.3 | 0 | 6 | 125 |
| Plasminogen | | | | | | | | | |
| Activator | | | | | | | | | |
| Receptor(uPAR) | | | | | | | | | |
| Vascular Cell | ng/mL | 366 | 1200 | 612 | 214 | 561 | 303 | 1510 | 126 |
| Adhesion Molecule- | | | | | | | | | |
| 1 (VCAM-1) | | | | | | | | | |
| Vascular | pg/mL | 282 | 659 | 430 | 99 | 414 | 235 | 804 | 126 |
| Endothelial | | | | | | | | | |
| Growth Factor | | | | | | | | | |
| (VEGF) | | | | | | | | | |
| Vascular | ng/mL | <LOW> | 11 | 7.0 | 2.9 | 5.6 | 3 | 14 | 41 |
| Endothelial | | | | | | | | | |
| Growth Factor | | | | | | | | | |
| B(VEGF-B) | | | | | | | | | |
| Vascular | ng/mL | 2.2 | 5.8 | 3.9 | 1.0 | 3.8 | 2 | 9 | 126 |
| Endothelial | | | | | | | | | |
| Growth Factor C | | | | | | | | | |
| (VEGF-C) | | | | | | | | | |
| Vascular | pg/mL | <LOW> | 1320 | 634 | 289 | 606 | 177 | 1620 | 116 |
| Endothelial | | | | | | | | | |
| Growth Factor | | | | | | | | | |
| D(VEGF-D) | | | | | | | | | |
| Vascular | pg/mL | <LOW> | 175 | 68 | 40 | 70 | 27 | 285 | 102 |
| Endothelial | | | | | | | | | |
| Growth Factor | | | | | | | | | |
| Receptor 1 | | | | | | | | | |
| (VEGFR-1) | | | | | | | | | |
| Vascular | ng/mL | 3.7 | 7.9 | 5.8 | 1.2 | 5.8 | 2 | 9 | 126 |
| Endothelial | | | | | | | | | |
| Growth Factor | | | | | | | | | |
| Receptor 2 | | | | | | | | | |
| (VEGFR-2) | | | | | | | | | |
| Vascular | ng/mL | 12 | 89 | 44 | 19 | 42 | 8 | 92 | 126 |
| Endothelial | | | | | | | | | |
| Growth Factor | | | | | | | | | |
| Receptor 3 | | | | | | | | | |
| (VEGFR-3) | | | | | | | | | |
| Vitamin K- | ug/ml | 16 | 35 | 23 | 5.8 | 22 | 14 | 54 | 126 |
| Dependent Protein | | | | | | | | | |
| S (VKDPS) | | | | | | | | | |
| Vitronectin | ug/ml | 844 | 2530 | 1791 | 420 | 1780 | 745 | 2870 | 126 |
| von Willebrand | ug/mL | 13 | 93 | 40 | 20 | 37 | 9 | 117 | 126 |
| Factor (vWF) | | | | | | | | | |
| YKL-40 | ng/mL | 17 | 88 | 35 | 22 | 28 | 15 | 173 | 126 |

In total, 18 confirmed HIBM (hereditary inclusion body myopathy) patient serum samples from the phase 1 clinical trial were used to test and identify biomarkers that were different in protein levels from normal serum samples.

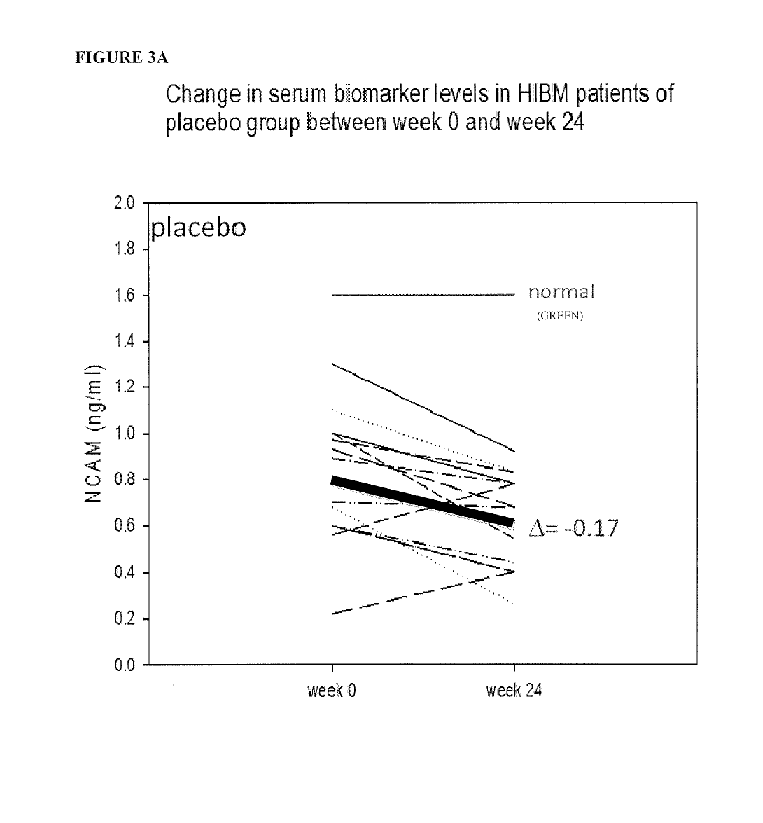

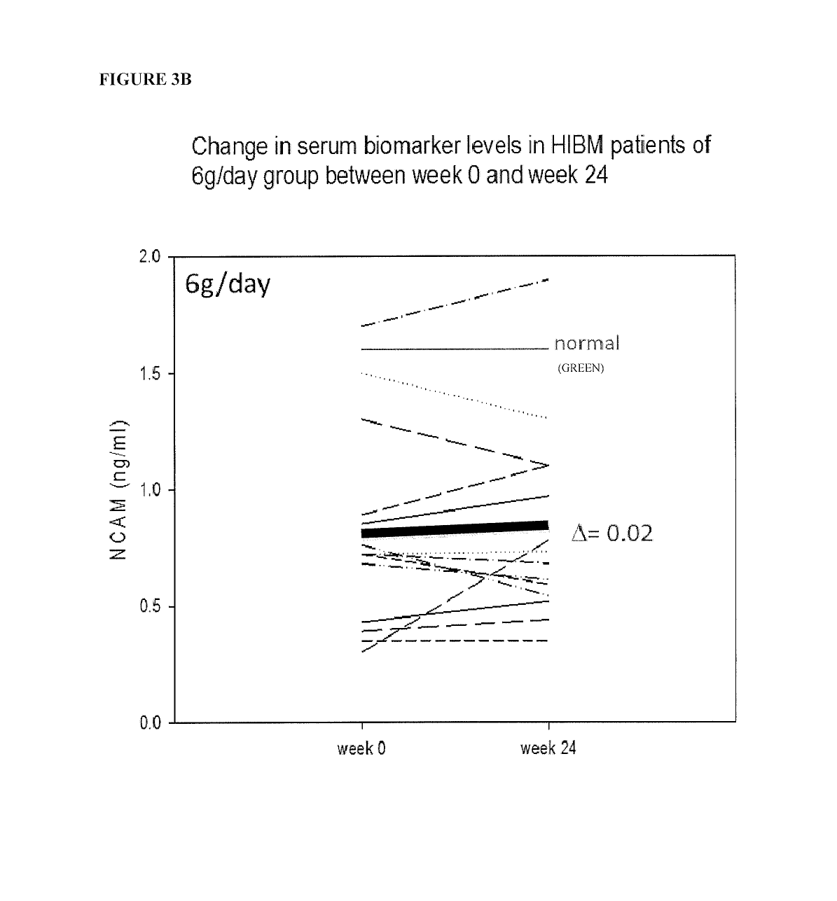

Data from the patient samples were compared to 126 “clinically normal” serum samples using Wilcoxon Rank Sum test. Of the 258 analytes tested, as a first cut 134 analytes had a p value of <0.05 (see FIG. 3 of U.S. Provisional Application Ser. No. 61/779,929 filed on Mar. 13, 2013), which are also those biomarkers in Table 2 underlined and bolded. From the 134 analytes, as a second cut 31 of them had >4 fold difference in the medians and 6 others might be related to HIBM. As a third cut, additional analyses by pathway distinction were performed (20 analytes showing relevant biology to HIBM were underlined and bolded in Tables 3-6 below): muscle inflammation/fibrosis, muscle & nerve development, muscle damage and other markers related to muscle.

TABLE 3
Muscle Inflammation and Fibrosis

| | | Fold | | | |
|---|---|---|---|---|---|
| Analyte | Symbol | Changes | p-value* | Primary Pathway | Note |
| Epithelial-Derived | ENA-78 | 4.5 | <0.001 | Inflammation | |
| Neutrophil-Activating | | | | | |
| Protein 78 | | | | | |
| CD40 Ligand | CD40-L | 24.1 | <0.001 | Inflammation | |
| Interleukin-3 | IL-3 | 10 | <0.001 | Inflammation | Response to chronic |
| | | | | | inflammation |
| Interleukin-5 | IL-5 | 9.6 | <0.001 | Inflammation | Response to chronic |
| | | | | | inflammation |
| Interleukin-7 | IL-7 | 8 | <0.001 | Inflammation | |
| Interleukin-8 | IL-8 | −5.3 | <0.001 | Inflammation | 2 fold reduction in median, |
| | | | | | 5.3 reduction in mean |
| Latency-Associated | LAP TGF-b1 | 10.1 | <0.001 | Inflammation | TGF beta important role in |
| Peptide of | | | | | fibrosis and modifier in |
| Transforming Growth | | | | | muscular dystrophy |
| Factor beta 1 | | | | | |
| Macrophage Colony- | M-CSF | 5.2 | <0.001 | Inflammation | Response to chronic |
| Stimulating Factor 1 | | | | | inflammation, similar |
| | | | | | pathway as interleukins |
| Macrophage | MIP-3 Alpha | −4.2 | <0.001 | Inflammation | Similar pathway to |
| Inflammatory Protein-3 | | | | | interleukins |
| alpha | | | | | |
| Myeloperoxidase | MPO | 3.9 | <0.001 | Inflammation | 3.8 fold increase in median, |
| | | | | | 3.9 fold increase in mean. |
| | | | | | Might be used to measure |
| | | | | | muscle damage due to |
| | | | | | neutrophil infiltration |
| T-Cell-Specific Protein | RANTES | 23.8 | <0.001 | Inflammation | Elevated in mdx diaphragm |
| RANTES | | | | | |
| Vascular Endothelial | VEGF-C | 8.9 | <0.001 | Inflammation/nerve | Similar to PDGF; elevated |
| Growth Factor C | | | | development | in DMD; improve muscle |
| | | | | | repair and reduce fibrosis |
| Agouti-Related | AgRP | ND | ND | Neuroendocrine | Undetectable in all HIBM |
| Protein | | <54 pg/mL | | response to | but detectable in all normal |
| | | | | inflammation | |
| Thrombospondin-1 | THBS1 | 14.3 | <0.001 | Inflammation/fibrosis | Binds to ECM proteins; |
| | | | | | chemotactic factors for |
| | | | | | inflammation in some |
| | | | | | muscle diseases |
| Platelet-Derived | PDGF-BB | 54.4 | <0.001 | Inflammation/fibrosis | Elevated in DMD and |
| Growth Factor BB | | | | | PDGF-receptor increased |
| | | | | | in regenerating muscles of |
| | | | | | mdx mice |
| Matrix | MMP-1 | 5.2 | <0.001 | Fibrosis | 3.5 fold increase in median, |
| Metalloproteinase-1 | | | | | 5.2 increase in mean |
| Matrix | MMP-3 | 3.8 | <0.001 | Fibrosis | Did not meet the 4-folds |
| Metalloproteinase-3 | | | | | cutoff criteria |
| Matrix | MMP-9, total | 4.4 | <0.001 | Fibrosis | Increased in DMD |
| Metalloproteinase-9, | | | | | |
| total | | | | | |

*Wilcoxon rank sum test

TABLE 4
Muscle and Nerve Development

| | | Fold | | Primary | |
|---|---|---|---|---|---|
| Analyte | Symbol | Changes | p-value* | Pathway | Note |
| Brain-Derived | BDNF | 38.8 | <0.001 | Nerve & muscle | Striatum BDNF decreased in |
| Neurotrophic Factor | | | | growth | mdx mice |
| Chromogranin-A | CgA | 19.7 | <0.001 | Nerve | |
| | | | | development | |
| Receptor tyrosine- | ErbB3 | −5 | <0.001 | Nerve | Neuregulin family essential for |
| protein kinase erbB-3 | | | | development | neuromuscular junction and |
| | | | | | myogenesis |
| Neuron-Specific | NSE | −7.7 | <0.001 | Nerve | Might be an indicator of |
| Enolase | | | | development/function | neuronal regeneration |
| Neural cell adhesion | NCAM | −2.1 | <0.001 | Nerve & muscle | The data might corroborate the |
| molecule | | | | function | muscle NCAM Western blot |
| | | | | | data |
| Epidermal Growth | EGF | 4.8 | <0.001 | Cell growth | More useful to look at erbB-3 |
| Factor | | | | | |
| Fibroblast Growth | FGF-4 | ND | ND | Cell growth | Undetectable in all HIBM but |
| Factor 4 | | <44 pg/mL | | | detectable in all normal. Might |
| | | | | | have some biological meaning. |
| | | | | | Other FGFs elevated in DMD |
| | | | | | and potential role in fibrosis |
| Kallikrien5 | KLK5 | 3.87 | <0.001 | Serine protease | Did not meet the 4-folds cutoff |
| | | | | | criteria |
| Plasminogen Activator | PAI-1 | 7.3 | <0.001 | Serine protease | Inhibition improves muscle |
| Inhibitor 1 | | | | inhibitor | regeneration |

*Wilcoxon rank sum test

TABLE 5
Muscle Damage Markers

| | | Fold | | Primary | |
|---|---|---|---|---|---|
| Analyte | Symbol | Changes | p-value* | Pathway | Note |
| Serum Glutamic | SGOT | 4.2 | <0.001 | Cell damage | Blood test already covers |
| Oxaloacetic | | | | | many markers for |
| Transaminase | | | | | muscle/organ damage |
| Creatine Kinase-MB | CK-MB | 6.8 | <0.001 | Muscle damage | Already assayed in blood test |
| Myoglobin | MB | 7.5 | <0.001 | Muscle damage | Another marker to assess |
| | | | | | muscle damage in addition to |
| | | | | | CK |

*Wilcoxon rank sum test

TABLE 6
Other Related Markers

| | | Fold | | Primary | |
|---|---|---|---|---|---|
| Analyte | Symbol | Changes | p-value* | Pathway | Note |
| N-terminal prohormone | NT proBMP | 5.6 | <0.001 | Biomarker for | Too many “low” in the normal |
| of brain natriuretic | | | | cardiovascular | and the range for many HIBM |
| peptide | | | | risk | samples is below least |
| | | | | | detectable dose (LDD). The |
| | | | | | difference between median and |
| | | | | | mean in normal is 5 times. |
| Protein S100-A4 | S100-A4 | 4 | <0.001 | Ca binding | Activates MMPs and nerve |
| | | | | protein | development, mRNA |
| | | | | | expression increased in DMD |
| Protein S100-A6 | S100-A6 | 3.6 | <0.001 | Ca binding | Did not meet the 4-folds cutoff |
| | | | | protein | criteria |
| Insulin-like Growth | IGFBP-6 | 5.1 | <0.001 | Tissue | IGF important for muscle |
| Factor Binding | | | | morphology | growth and mass |
| Protein 6 | | | | | |
| Thyroglobulin | TG | −4 | <0.001 | Tissue | 3.7 fold increase in median, 4 |
| | | | | morphology | fold increase in mean |
| Amphiregulin | AR | −4.4 | <0.001 | Related to EGF | Too many “low” in the normal |
| | | | | and TGF-a; | and the range for many HIBM |
| | | | | growth factor | samples is below least |
| | | | | for fibroblasts, | detectable dose (LDD). |
| | | | | Schwann cells | |
| Cortisol | Cortisol | 4.7 | <0.001 | Stress response | Might be elevated in many |
| | | | | Suppress | conditions unrelated to muscle |
| | | | | immune system | diseases |

*Wilcoxon rank sum test

A total of 37 markers with p value <0.05 and >4 fold difference in medians were identified (Table 7). These markers were further grouped into 3-4 major biological pathways using bioinformative software in which 20 of them showed relevant biology to HIBM. The list can be further narrowed down to: a total of 18 markers in which >50% of normal samples are above “least detectable dose (LDD)” or more than 5 fold changes in median (Table 8). A total of 11 markers had p<0.05 and significantly greater than 5 fold changes in median; >80% of the patient samples are above 5 times the LDD, showing the robustness of the assay (Table 9). From the box graph analysis, the distribution of normal vs. HIBM was wide apart, partly due to the large difference in median ratio.

TABLE 7
37 markers w/p <0.05 and greater or close to 4 fold changes in median, therefore
having a median ratio of HIBM/normal at >4 or <0.25

| (Rows in grey: HIBM patients) | | | | | median ratio: |
|---|---|---|---|---|---|
| (Rows in white: normal) | mean | median | SD | p value | HIBM/normal |
| Agouti-Related Protein (AgRP) | <LOW> | <LOW> | <LOW> | | |
| Agouti-Related Protein (AgRP) | 196.44 | 183 | 85.8 | N/A | N/A |
| Amphiregulin (AR) | 161.81 | 113.5 | 102.9 | | |
| Amphiregulin (AR) | 505 | 484 | 219.88 | 1.83E−07 | 0.23 |
| Brain-Derived Neurotrophic Factor | 14.91 | 15.5 | 7.86 | | |
| (BDNF) | | | | | |
| Brain-Derived Neurotrophic Factor | 0.57 | 0.4 | 0.69 | 8.30E−07 | 38.8 |
| (BDNF) | | | | | |
| CD40 Ligand (CD40-L) | 0.86 | 0.47 | 0.82 | | |
| CD40 Ligand (CD40-L) | 0.03 | 0.02 | 0.06 | 0.00065 | 24.09 |
| Chromogranin-A (CgA) | 270.67 | 248 | 146.43 | | |
| Chromogranin-A (CgA) | 78.99 | 12.6 | 111.66 | 8.10E−05 | 19.68 |
| Cortisol (Cortisol) | 494.83 | 438.5 | 209.32 | | |
| Cortisol (Cortisol) | 101.08 | 94.1 | 51.43 | 5.30E−07 | 4.66 |
| Creatine Kinase-MB (CK-MB) | 6.57 | 5.45 | 4.77 | | |
| Creatine Kinase-MB (CK-MB) | 1.09 | 0.8 | 0.93 | 0.00019 | 6.83 |
| Epidermal Growth Factor (EGF) | 78.23 | 22.5 | 90.62 | | |
| Epidermal Growth Factor (EGF) | 11.81 | 4.66 | 43.9 | 0.00818 | 4.83 |
| Epithelial-Derived Neutrophil- | 1.77 | 1.3 | 1.21 | | |
| Activating Protein 78 (ENA-78) | | | | | |
| Epithelial-Derived Neutrophil- | 0.38 | 0.29 | 0.39 | 0.00019 | 4.45 |
| Activating Protein 78 (ENA-78) | | | | | |
| Fibroblast Growth Factor 4 (FGF-4) | <LOW> | <LOW> | <LOW> | | |
| Fibroblast Growth Factor 4 (FGF-4) | 209.62 | 201 | 107.22 | N/A | N/A |
| Insulin-like Growth Factor Binding | 523 | 535 | 227.78 | | |
| Protein 6 (IGFBP-6) | | | | | |
| Insulin-like Growth Factor Binding | 125.97 | 104.5 | 81.83 | 1.40E−06 | 5.12 |
| Protein 6 (IGFBP-6) | | | | | |
| Interleukin-3 (IL-3) | 0.11 | 0.09 | 0.08 | | |
| Interleukin-3 (IL-3) | 0.01 | 0.01 | 0.01 | 0.00012 | 10.03 |
| Interleukin-5 (IL-5) | 23.1 | 21 | 20.1 | | |
| Interleukin-5 (IL-5) | 2.91 | 2.18 | 2.61 | 0.001 | 9.63 |
| Interleukin-7 (IL-7) | 112.13 | 112.5 | 67.84 | | |
| Interleukin-7 (IL-7) | 21.15 | 14 | 25.51 | 0.0001 | 8.04 |
| Interleukin-8 (IL-8) | 9.96 | 10.5 | 3.21 | | |
| Interleukin-8 (IL-8) | 51.32 | 20.1 | 68.95 | 8.60E−10 | 0.52 |
| Kallikrein 5 | 3.72 | 3.35 | 1.57 | | |
| Kallikrein 5 | 0.96 | 0.89 | 0.51 | 2.40E−05 | 3.76 |
| Latency-Associated Peptide of | 9.31 | 10 | 2.61 | | |
| Transforming Growth Factor beta 1 | | | | | |
| (LAP TGF-b1 | | | | | |
| Latency-Associated Peptide of | 1.09 | 0.99 | 0.54 | 2.70E−10 | 10.13 |
| Transforming Growth Factor beta 1 | | | | | |
| (LAP TGF-b1) | | | | | |
| Macrophage Colony-Stimulating | 0.52 | 0.48 | 0.18 | | |
| Factor 1 (M-CSF) | | | | | |
| Macrophage Colony-Stimulating | 0.1 | 0.09 | 0.03 | 7.10E−08 | 5.22 |
| Factor 1 (M-CSF) | | | | | |
| Macrophage Inflammatory Protein- | 22.5 | 17.5 | 8.45 | | |
| 3 alpha (MIP-3 alpha) | | | | | |
| Macrophage Inflammatory Protein- | 87.3 | 72.8 | 87.51 | 6.80E−11 | 0.24 |
| 3 alpha (MIP-3 alpha) | | | | | |
| Matrix Metalloproteinase-1 (MMP- | 5.94 | 3.3 | 5.22 | | |
| 1) | | | | | |
| Matrix Metalloproteinase-1 (MMP- | 1.15 | 0.95 | 0.96 | 0.00149 | 3.46 |
| 1) | | | | | |
| Matrix Metalloproteinase-3 (MMP- | 11.36 | 11.5 | 5.88 | | |
| 3) | | | | | |
| Matrix Metalloproteinase-3 (MMP- | 4.25 | 3.05 | 3.78 | 0.00011 | 3.77 |
| 3) | | | | | |
| Matrix Metalloproteinase-9, total | 493.44 | 501 | 191.67 | | |
| (MMP-9, total) | | | | | |
| Matrix Metalloproteinase-9, total | 119.36 | 114 | 54.23 | 3.10E−07 | 4.39 |
| (MMP-9, total) | | | | | |
| Myeloperoxidase (MPO) | 783.18 | 592 | 571.62 | | |
| Myeloperoxidase (MPO) | 199.48 | 158 | 138.16 | 0.00087 | 3.75 |
| Myoglobin | 120.67 | 85.5 | 74.72 | | |
| Myoglobin | 14.96 | 11.35 | 11.91 | 2.00E−05 | 7.53 |
| N-terminal prohormone of brain | 344.76 | 315 | 259.74 | | |
| natriuretic peptide (NT proBNP) | | | | | |
| N-terminal prohormone of brain | 287.81 | 56.25 | 1236.2 | 0.72661 | 5.6 |
| natriuretic peptide (NT proBNP) | | | | | |
| Neuron-Specific Enolase (NSE) | 1.73 | 1.35 | 1.29 | | |
| Neuron-Specific Enolase (NSE) | 13.96 | 10.46 | 11.33 | 6.70E−20 | 0.13 |
| Neuronal Cell Adhesion Molecule | 0.72 | 0.57 | 0.47 | | |
| (Nr-CAM) | | | | | |
| Neuronal Cell Adhesion Molecule | 1.61 | 1.45 | 0.89 | 4.30E−07 | 0.39 |
| (Nr-CAM) | | | | | |
| Plasminogen Activator Inhibitor 1 | 169.89 | 171.5 | 75.22 | | |
| (PAI-1) | | | | | |
| Plasminogen Activator Inhibitor 1 | 32.1 | 23.4 | 28.96 | 7.00E−07 | 7.33 |
| (PAI-1) | | | | | |
| Platelet-Derived Growth Factor BB | 9775 | 9085 | 5091.84 | | |
| (PDGF-BB) | | | | | |
| Platelet-Derived Growth Factor BB | 271.3 | 167 | 324.68 | 6.10E−07 | 54.4 |
| (PDGF-BB) | | | | | |
| Protein S100-A4 (S100-A4) | 24.48 | 18 | 24.42 | | |
| Protein S100-A4 (S100-A4) | 6.58 | 4.56 | 7.81 | 0.01004 | 3.95 |
| Protein S100-A6 (S100-A6) | 21.96 | 21.5 | 10.86 | | |
| Protein S100-A6 (S100-A6) | 6.88 | 6 | 5.06 | 2.40E−05 | 3.58 |
| Receptor tyrosine-protein kinase | 0.75 | 0.46 | 0.69 | | |
| erbB-3 (ErbB3) | | | | | |
| Receptor tyrosine-protein kinase | 2.15 | 2.29 | 0.85 | 7.00E−08 | 0.2 |
| erbB-3 (ErbB3) | | | | | |
| Serum Glutamic Oxaloacetic | 10.12 | 9.25 | 3.88 | | |
| Transaminase (SGOT) | | | | | |
| Serum Glutamic Oxaloacetic | 2.46 | 2.2 | 0.97 | 2.70E−07 | 4.21 |
| Transaminase (SGOT) | | | | | |
| T-Cell-Specific Protein RANTES | 23.26 | 25 | 11.73 | | |
| (RANTES) | | | | | |
| T-Cell-Specific Protein RANTES | 1.44 | 1.05 | 1.73 | 6.30E−07 | 23.81 |
| (RANTES) | | | | | |
| Thrombospondin-1 | 8488.11 | 8355 | 4265.59 | | |
| Thrombospondin-1 | 875.94 | 584 | 1155.01 | 1.10E−06 | 14.31 |
| Thyroglobulin (TG) | 14.33 | 9.4 | 15.77 | | |
| Thyroglobulin (TG) | 57.16 | 33.55 | 98.25 | 1.60E−05 | 0.28 |
| Vascular Endothelial Growth Factor | 29.78 | 33.5 | 8.09 | | |
| C (VEGF-C) | | | | | |
| Vascular Endothelial Growth Factor | 3.89 | 3.75 | 1.04 | 2.30E−10 | 8.93 |
| C (VEGF-C) | | | | | |

TABLE 8
reduced to 18 markers after removing markers that have >50% of normal samples at
least detectable dose or less than 5 fold changes in median

| (Rows in grey: HIBM patients) | | | | | median ratio: |
|---|---|---|---|---|---|
| (Rows in white: normal) | mean | median | SD | p value | HIBM/normal |
| Brain-Derived Neurotrophic Factor | 14.91 | 15.5 | 7.86 | | |
| (BDNF) | | | | | |
| Brain-Derived Neurotrophic Factor | 0.57 | 0.4 | 0.69 | 8.30E−07 | 38.8 |
| (BDNF) | | | | | |
| CD40 Ligand (CD40-L | 0.86 | 0.47 | 0.82 | | |
| CD40 Ligand (CD40-L | 0.03 | 0.02 | 0.06 | 0.00065 | 24.09 |
| Chromogranin-A (CgA | 270.67 | 248 | 146.43 | | |
| Chromogranin-A (CgA | 78.99 | 12.6 | 111.66 | 8.10E−05 | 19.68 |
| Creatine Kinase-MB (CK-MB | 6.57 | 5.45 | 4.77 | | |
| Creatine Kinase-MB (CK-MB | 1.09 | 0.8 | 0.93 | 0.00019 | 6.83 |
| Insulin-like Growth Factor Binding | 523 | 535 | 227.78 | | |
| Protein 6 (IGFBP-6) | | | | | |
| Insulin-like Growth Factor Binding | 125.97 | 104.5 | 81.83 | 1.40E−06 | 5.12 |
| Protein 6 (IGFBP-6) | | | | | |
| Interleukin-3 (IL-3 | 0.11 | 0.09 | 0.08 | | |
| Interleukin-3 (IL-3 | 0.01 | 0.01 | 0.01 | 0.00012 | 10.03 |
| Interleukin-5 (IL-5 | 23.1 | 21 | 20.1 | | |
| Interleukin-5 (IL-5 | 2.91 | 2.18 | 2.61 | 0.001 | 9.63 |
| Latency-Associated Peptide of | 9.31 | 10 | 2.61 | | |
| Transforming Growth Factor beta 1 | | | | | |
| (LAP TGF-b1) | | | | | |
| Latency-Associated Peptide of | 1.09 | 0.99 | 0.54 | 2.70E−10 | 10.13 |
| Transforming Growth Factor beta 1 | | | | | |
| (LAP TGF-b1) | | | | | |
| Macrophage Colony-Stimulating | 0.52 | 0.48 | 0.18 | | |
| Factor 1 (M-CSF) | | | | | |
| Macrophage Colony-Stimulating | 0.1 | 0.09 | 0.03 | 7.10E−08 | 5.22 |
| Factor 1 (M-CSF) | | | | | |
| Matrix Metalloproteinase-9, total | 493.44 | 501 | 191.67 | | |
| (MMP-9, total) | | | | | |
| Matrix Metalloproteinase-9, total | 119.36 | 114 | 54.23 | 3.10E−07 | 4.39 |
| (MMP-9, total) | | | | | |
| Myoglobin | 120.67 | 85.5 | 74.72 | | |
| Myoglobin | 14.96 | 11.35 | 11.91 | 2.00E−05 | 7.53 |
| Neuron-Specific Enolase (NSE) | 1.73 | 1.35 | 1.29 | | |
| Neuron-Specific Enolase (NSE) | 13.96 | 10.46 | 11.33 | 6.70E−20 | 0.13 |
| Plasminogen Activator Inhibitor 1 | 169.89 | 171.5 | 75.22 | | |
| (PAI-1) | | | | | |
| Plasminogen Activator Inhibitor 1 | 32.1 | 23.4 | 28.96 | 7.00E−07 | 7.33 |
| (PAI-1) | | | | | |
| Platelet-Derived Growth Factor BB | 9775 | 9085 | 5091.84 | | |
| (PDGF-BB) | | | | | |
| Platelet-Derived Growth Factor BB | 271.3 | 167 | 324.68 | 6.10E−07 | 54.4 |
| (PDGF-BB) | | | | | |
| Receptor tyrosine-protein kinase | 0.75 | 0.46 | 0.69 | | |
| erbB-3 (ErbB3) | | | | | |
| Receptor tyrosine-protein kinase | 2.15 | 2.29 | 0.85 | 7.00E−08 | 0.2 |
| erbB-3 (ErbB3) | | | | | |
| T-Cell-Specific Protein RANTES | 23.26 | 25 | 11.73 | | |
| (RANTES) | | | | | |
| T-Cell-Specific Protein RANTES | 1.44 | 1.05 | 1.73 | 6.30E−07 | 23.81 |
| (RANTES) | | | | | |
| Thrombospondin-1 | 8488.11 | 8355 | 4265.59 | | |
| | | | 1155.01 | | |
| Thrombospondin-1 | 875.94 | 584 | 8.09 | 1.10E−06 | 14.31 |
| Vascular Endothelial Growth Factor | 29.78 | 33.5 | 1.04 | | |
| C (VEGF-C) | | | | | |
| Vascular Endothelial Growth Factor | 3.89 | 3.75 | | 2.30E−10 | 8.93 |
| C (VEGF-C) | | | | | |

TABLE 9
11 markers w/p <0.05 and significantly greater than 5 fold changes in median. Also,
>80% of the samples above 5 times the least detectable dose (LDD)

| (Rows in grey: HIBM patients) | | | | | median ratio: |
|---|---|---|---|---|---|
| (Rows in white: normal) | mean | median | SD | p value | HIBM/normal |
| Brain-Derived Neurotrophic Factor (BDNF) | 14.91 | 15.5 | 7.86 | | |
| Brain-Derived Neurotrophic Factor (BDNF) | 0.57 | 0.4 | 0.69 | 8.30E−07 | 38.8 |
| CD40 Ligand (CD40-L) | 0.86 | 0.47 | 0.82 | | |
| CD40 Ligand (CD40-L) | 0.03 | 0.02 | 0.06 | 0.00065 | 24.09 |
| Chromogranin-A (CgA) | 270.67 | 248 | 146.43 | | |
| Chromogranin-A (CgA) | 78.99 | 12.6 | 111.66 | 8.10E−05 | 19.68 |
| Latency-Associated Peptide of | 9.31 | 10 | 2.61 | | |
| Transforming Growth Factor beta 1 (LAP | | | | | |
| TGF-b1) | | | | | |
| Latency-Associated Peptide of | 1.09 | 0.99 | 0.54 | 2.70E−10 | 10.13 |
| Transforming Growth Factor beta 1 (LAP | | | | | |
| TGF-b1) | | | | | |
| Matrix Metalloproteinase-9, total (MMP-9, | 493.44 | 501 | 191.67 | | |
| total) | | | | | |
| Matrix Metalloproteinase-9, total (MMP-9, | 119.36 | 114 | 54.23 | 3.10E−07 | 4.39 |
| total) | | | | | |
| Myoglobin | 120.67 | 85.5 | 74.72 | | |
| Myoglobin | 14.96 | 11.35 | 11.91 | 2.00E−05 | 7.53 |
| Plasminogen Activator Inhibitor 1 (PAI-1) | 169.89 | 171.5 | 75.22 | | |
| Plasminogen Activator Inhibitor 1 (PAI-1) | 32.1 | 23.4 | 28.96 | 7E−07 | 7.33 |
| Platelet-Derived Growth Factor BB (PDGF- | 9775 | 9085 | 5091.84 | | |
| BB) | | | | | |
| Platelet-Derived Growth Factor BB (PDGF- | 271.3 | 167 | 324.68 | 6.10E−07 | 54.4 |
| BB) | | | | | |
| Receptor tyrosine-protein kinase erbB-3 | 0.75 | 0.46 | 0.69 | | |
| (ErbB3) | | | | | |
| Receptor tyrosine-protein kinase erbB-3 | 2.15 | 2.29 | 0.85 | 7E−08 | 0.2 |
| (ErbB3) | | | | | |
| T-Cell-Specific Protein RANTES | 23.26 | 25 | 11.73 | | |
| (RANTES) | | | | | |
| T-Cell-Specific Protein RANTES | 1.44 | 1.05 | 1.73 | 6.30E−07 | 23.81 |
| (RANTES) | | | | | |
| Thrombospondin-1 | 8488.11 | 8355 | 4265.59 | | |
| Thrombospondin-1 | 875.94 | 584 | 1155.01 | 1.10E−06 | 14.31 |

The system, along with the results generated will facilitate the establishment of a panel of markers to monitor treatment response in HIBM. The invention is also useful for diagnostic purpose, such that, a panel of unique and specific serum markers that can be used to differentiate HIBM from other muscle diseases. In addition, the system, along with the results generated will enable identification of new drug targets in HIBM.

### Example 2

### Phase 2 Clinical Trial

The plan for further work is to test phase 2 patient samples at both baseline (before treatment), after 24 weeks of treatment and 48 weeks of treatment. As used herein, the term “baseline level” refers to a standard control for “normal” levels (i.e., patients without disease), but can also be comparative, e.g., where low baseline levels is compared to the levels of other subjects having the disease.

Forty-seven (47) HIBM patient serum samples collected at week 24 from phase 2 study were tested using the Human Discovery MAP250+ v2.0 quantitative immunoassay system developed by Myriad RBM. The results were compared to the serum samples of the same 47 HIBM patients collected at week 0 (baseline) which were conducted earlier with the same immunoassay system.

Patients were unblinded and the results were organized into the 3 assigned dosage groups, placebo (n=14), 3 g/day (n=17) and 6 g/day (n=14). The major focus of the analysis was 1) to assess changes (delta, A) in levels of analytes between baseline and week 24 in each dosage group, 2) whether the changes correlate to dosage levels.

Two patients did not provide serum samples at week 24 and therefore, the final number of patients being analyzed was 45.

Previous analysis of the baseline serum samples identified 31 potential analytes that showed significant changes (>4 folds change in mean or median with a p-value <0.05) in the patients compared to 126 normal controls. The current analysis also included an additional analyte, Neural Cell Adhesion Molecule (NCAM) that was thought to be an important biomarker for sialylation in HIBM. See FIG. 9 of U.S. Provisional Application Ser. No. 61/779,929 filed on Mar. 13, 2013 which shows 243 analytes (markers) in the Human Discovery MAP250 quantitative immunoassay system tested in 47 HIBM patients. Data from the patient samples were compared to 126 “clinically normal” serum samples using Wilcoxon Rank Sum test. Of the 243 analytes tested, as a first cut 140 analytes had a p value of <0.05 (see FIG. 10 of U.S. Provisional Application Ser. No. 61/779,929 filed on Mar. 13, 2013, which are also those biomarkers in Table 10a and Table 10b). From the 140 analytes, as a second cut 31 of them had >4 fold difference in the medians and 6 others might be related to HIBM (see FIG. 11 of U.S. Provisional Application Ser. No. 61/779,929 filed on Mar. 13, 2013, which are also those biomarkers in Table 11). As a third cut, additional analyses by pathway distinction were performed, and 16 analytes of the 31 had >50% of HIBM and normal samples are above LDD or greater than 5 fold changes in median (see FIG. 12 of U.S. Provisional Application Ser. No. 61/779,929 filed on Mar. 13, 2013, which are also those biomarkers in Table 12):

TABLE 10a
Phase II Clinical Trial - 140 analytes (biomarkers) which had a p value of <0.05 (126 Normal Controls)

| | | | | | | | | | change in | change in |
|---|---|---|---|---|---|---|---|---|---|---|
| | | | | | | | | | mean | median |
| | | Normal | Normal | Normal | | | Positive | p- | (HIBM/ | (HIBM/ |
| 140 markers with p < 0.05. | Units | Mean | SD | Median | Min | Max | Count | value | nomral) | normal) |
| 6Ckine | pg/mL | 536 | 182 | 520 | 201 | 1200 | 126 | 0.0001 | 0.85 | 0.89 |
| Aldose Reductase | ng/mL | 3.4 | 1.7 | 3.4 | 0 | 12 | 125 | 0.0000 | 3.45 | 2.94 |
| Alpha-1-Microglobulin | ug/mL | 11 | 2.9 | 11 | 6 | 23 | 126 | 0.0467 | 0.93 | 0.92 |
| (A1Micro) | | | | | | | | | | |
| Alpha-2-Macroglobulin | mg/mL | 3.2 | 1.8 | 2.9 | 1 | 8 | 126 | 0.0000 | 0.47 | 0.52 |
| (A2Macro) | | | | | | | | | | |
| Angiogenin | ng/mL | 623 | 191 | 588 | 260 | 1600 | 126 | 0.0000 | 0.66 | 0.64 |
| Angiopoietin-2 (ANG-2) | ng/mL | 3.2 | 1.5 | 3.0 | 0 | 9 | 126 | 0.0000 | 1.32 | 1.38 |
| Angiotensin-Converting Enzyme | ng/mL | 92 | 30 | 87 | 43 | 197 | 126 | 0.0000 | 0.71 | 0.70 |
| (ACE) | | | | | | | | | | |
| Apolipoprotein(a) (Lp(a)) | ug/mL | 781 | 875 | 506 | 1 | 3760 | 122 | 0.0000 | 0.39 | 0.45 |
| Apolipoprotein A-I (Apo A-I) | mg/mL | 1.3 | 0.36 | 1.3 | 1 | 3 | 126 | 0.0000 | 1.25 | 1.25 |
| Apolipoprotein A-II (Apo A-II) | ng/mL | 534 | 274 | 448 | 189 | 1560 | 126 | 0.0000 | 0.62 | 0.72 |
| Apolipoprotein A-IV (Apo A-IV) | ug/mL | 38 | 22 | 34 | 5 | 134 | 126 | 0.0000 | 0.44 | 0.33 |
| Apolipoprotein B (Apo B) | ug/mL | 2013 | 714 | 1830 | 835 | 5130 | 126 | 0.0000 | 0.65 | 0.76 |
| Apolipoprotein C-III (Apo C-III) | ug/mL | 163 | 106 | 135 | 52 | 758 | 126 | 0.0000 | 1.43 | 1.52 |
| Apolipoprotein D (Apo D) | ug/mL | 192 | 71 | 178 | 52 | 481 | 126 | 0.0000 | 0.65 | 0.69 |
| Apolipoprotein H (Apo H) | ug/mL | 296 | 87 | 282 | 143 | 589 | 126 | 0.0000 | 0.84 | 0.88 |
| AXL Receptor Tyrosine Kinase | ng/mL | 9.7 | 3.0 | 9.5 | 2 | 20 | 126 | 0.0044 | 1.17 | 1.15 |
| (AXL) | | | | | | | | | | |
| B cell-activating factor (BAFF) | pg/mL | 684 | 191 | 654 | 259 | 1380 | 126 | 0.0000 | 1.22 | 1.27 |
| B Lymphocyte Chemoattractant | pg/mL | 19 | 42 | 8.6 | 6 | 245 | 67 | 0.0006 | 3.63 | 8.17 |
| (BLC) | | | | | | | | | | |
| Beta-2-Microglobulin (B2M) | ug/mL | 1.7 | 0.47 | 1.6 | 1 | 3 | 126 | 0.0000 | 0.77 | 0.80 |
| Brain-Derived Neurotrophic | ng/mL | 0.57 | 0.69 | 0.40 | 0 | 7 | 126 | 0.0000 | 34.58 | 50.06 |
| Factor (BDNF) | | | | | | | | | | |
| C-Peptide | ng/mL | 1.4 | 1.5 | 1.1 | 0 | 12 | 126 | 0.0000 | 1.77 | 2.14 |
| Carcinoembryonic Antigen (CEA) | ng/mL | 2.2 | 2.6 | 1.6 | 0 | 28 | 124 | 0.0000 | 0.46 | 0.52 |
| Cathepsin D | ng/mL | 357 | 89 | 346 | 208 | 814 | 126 | 0.0001 | 0.81 | 0.81 |
| CD5 Antigen-like (CD5L) | ng/mL | 1624 | 647 | 1450 | 581 | 4580 | 126 | 0.0000 | 1.56 | 1.70 |
| CD 40 antigen (CD40) | ng/mL | 0.44 | 0.11 | 0.42 | 0 | 1 | 126 | 0.0000 | 2.10 | 2.13 |
| CD40 Ligand (CD40-L) | ng/mL | 0.031 | 0.064 | 0.019 | 0 | 0 | 54 | 0.0000 | 57.13 | 82.90 |
| Cellular Fibronectin (cFib) | ug/mL | 0.66 | 0.39 | 0.63 | 0 | 2 | 27 | 0.0000 | 3.23 | 2.92 |
| Chromogranin-A (CgA) | ng/mL | 79 | 113 | 13 | 5 | 372 | 52 | 0.0000 | 4.36 | 25.95 |
| Clusterin (CLU) | ug/mL | 302 | 84 | 286 | 112 | 577 | 126 | 0.0000 | 0.85 | 0.91 |
| Complement C3 (C3) | mg/mL | 2.2 | 0.58 | 2.1 | 1 | 4 | 126 | 0.0000 | 0.57 | 0.57 |
| Complement Factor H - Related | ug/mL | 3333 | 1375 | 3330 | 704 | 6810 | 126 | 0.0000 | 0.70 | 0.69 |
| Protein 1 (CFHR1) | | | | | | | | | | |
| Cortisol (Cortisol) | ng/mL | 101 | 52 | 94 | 10 | 250 | 123 | 0.0000 | 2.21 | 2.20 |
| Creatine Kinase-MB (CK-MB) | ng/mL | 1.1 | 0.93 | 0.80 | 0 | 6 | 125 | 0.0000 | 11.37 | 10.90 |
| Cystatin-C | ng/mL | 1035 | 286 | 980 | 330 | 1780 | 126 | 0.0000 | 0.78 | 0.83 |
| E-Selectin | ng/mL | 19 | 7.7 | 19 | 1 | 47 | 126 | 0.0000 | 0.46 | 0.43 |
| EN-RAGE | ng/mL | 15 | 11 | 11 | 3 | 67 | 126 | 0.0003 | 2.90 | 2.28 |
| Endoglin | ng/mL | 3.9 | 1.0 | 3.8 | 1 | 7 | 126 | 0.0000 | 0.84 | 0.90 |
| Endostatin | ng/mL | 79 | 19 | 77 | 33 | 144 | 126 | 0.0003 | 1.23 | 1.26 |
| Eotaxin-1 | pg/mL | 42 | 30 | 28 | 11 | 202 | 86 | 0.0000 | 4.90 | 7.39 |
| Epidermal Growth Factor (EGF) | pg/mL | 12 | 44 | 4.7 | 2 | 432 | 102 | 0.0000 | 23.55 | 53.22 |
| Epidermal Growth Factor | ng/mL | 4.4 | 0.62 | 4.4 | 3 | 6 | 126 | 0.0008 | 0.92 | 0.94 |
| Receptor (EGFR) | | | | | | | | | | |
| Epithelial-Derived Neutrophil- | ng/mL | 0.38 | 0.39 | 0.29 | 0 | 4 | 126 | 0.0000 | 6.42 | 6.85 |
| Activating Protein 78 (ENA-78) | | | | | | | | | | |
| Factor VII | ng/mL | 310 | 167 | 304 | 54 | 855 | 126 | 0.0016 | 1.26 | 1.21 |
| FASLG Receptor (FAS) | ng/mL | 10 | 12 | 8.0 | 2 | 134 | 123 | 0.0007 | 1.55 | 1.88 |
| Fatty Acid-Binding Protein, | ng/mL | 14 | 11 | 12 | 2 | 80 | 126 | 0.0001 | 1.50 | 1.60 |
| adipocyte (FABP, adipocyte) | | | | | | | | | | |
| Fetuin-A | ug/mL | 1269 | 363 | 1200 | 697 | 2490 | 126 | 0.0000 | 0.58 | 0.62 |
| Fibrinogen | mg/mL | 0.028 | 0.010 | 0.025 | 0 | 0 | 86 | 0.0000 | 2.48 | 2.51 |
| Galectin-3 | ng/mL | 11 | 4.1 | 10 | 3 | 27 | 125 | 0.0000 | 1.26 | 1.25 |
| Gelsolin | ug/mL | 66 | 14 | 65 | 38 | 114 | 126 | 0.0000 | 0.77 | 0.77 |
| Glucagon-like Peptide 1, total | pg/mL | 2.8 | 2.0 | 2.4 | 1 | 9 | 52 | 0.0011 | 6.09 | 4.71 |
| (GLP-1 total) | | | | | | | | | | |
| Glutathione S-Transferase Mu 1 | ng/mL | 4.7 | 3.2 | 4.2 | 1 | 14 | 82 | 0.0000 | 1.38 | 1.53 |
| (GST-M1) | | | | | | | | | | |
| Granulocyte Colony-Stimulating | pg/mL | 8.6 | 5.6 | 7.3 | 3 | 48 | 122 | 0.0433 | 1.16 | 1.20 |
| Factor (G-CSF) | | | | | | | | | | |
| Hepatocyte Growth Factor (HGF) | ng/mL | 3.1 | 2.1 | 2.6 | 0 | 11 | 123 | 0.0000 | 4.59 | 5.04 |
| Hepsin | pg/mL | 815 | 187 | 820 | 227 | 1360 | 126 | 0.0000 | 1.32 | 1.28 |
| Human Epidermal Growth Factor | ng/mL | 0.59 | 0.17 | 0.57 | 0 | 1 | 126 | 0.0002 | 0.81 | 0.80 |
| Receptor 2 (HER-2) | | | | | | | | | | |
| Immunoglobulin A (IgA) | mg/mL | 3.8 | 1.7 | 3.4 | 1 | 10 | 126 | 0.0000 | 0.57 | 0.58 |
| Immunoglobulin M (IgM) | mg/mL | 2.9 | 1.4 | 2.6 | 1 | 8 | 126 | 0.0000 | 0.65 | 0.57 |
| Insulin-like Growth Factor- | ng/mL | 80 | 49 | 70 | 16 | 278 | 126 | 0.0000 | 0.65 | 0.60 |
| Binding Protein 2 (IGFBP-2) | | | | | | | | | | |
| Insulin-like Growth Factor | ng/mL | 520 | 153 | 511 | 138 | 1000 | 126 | 0.0000 | 0.70 | 0.71 |
| Binding Protein 4 (IGFBP4) | | | | | | | | | | |
| Insulin-like Growth Factor | ng/mL | 148 | 30 | 145 | 78 | 250 | 126 | 0.0006 | 0.88 | 0.91 |
| Binding Protein 5 (IGFBP5) | | | | | | | | | | |
| Insulin-like Growth Factor | ng/mL | 126 | 82 | 105 | 9 | 420 | 126 | 0.0000 | 2.90 | 3.49 |
| Binding Protein 6 (IGFBP6) | | | | | | | | | | |
| Intercellular Adhesion Molecule 1 | ng/mL | 98 | 48 | 97 | 28 | 242 | 121 | 0.0002 | 1.26 | 1.25 |
| (ICAM-1) | | | | | | | | | | |
| Interferon gamma Induced Protein | pg/mL | 313 | 244 | 272 | 52 | 2360 | 126 | 0.0101 | 1.45 | 1.28 |
| 10 (IP-10) | | | | | | | | | | |
| Interferon-inducible T-cell alpha | pg/mL | 15 | 10 | 11 | 5 | 57 | 108 | 0.0001 | 3.88 | 3.82 |
| chemoattractant (ITAC) | | | | | | | | | | |
| Interleukin-1 alpha (IL-1 alpha) | ng/mL | 0.0021 | 0.0019 | 0.0018 | 0 | 0 | 85 | 0.0000 | 1.99 | 2.23 |
| Interleukin-1 receptor antagonist | pg/mL | 61 | 35 | 51 | 14 | 242 | 96 | 0.0000 | 2.87 | 3.11 |
| (IL-1ra) | | | | | | | | | | |
| Interleukin-2 receptor alpha (IL-2 | pg/mL | 1444 | 556 | 1390 | 566 | 3880 | 126 | 0.0000 | 1.64 | 1.71 |
| receptor alpha) | | | | | | | | | | |
| Interleukin-6 receptor (IL-6r) | ng/mL | 23 | 7.4 | 21 | 5 | 41 | 126 | 0.0000 | 1.30 | 1.34 |
| Interleukin-6 receptor subunit beta | ng/mL | 185 | 37 | 185 | 109 | 347 | 126 | 0.0003 | 0.89 | 0.88 |
| (IL-6R beta) | | | | | | | | | | |
| Interleukin-7 (IL-7) | pg/mL | 21 | 26 | 14 | 4 | 185 | 54 | 0.0463 | 0.59 | 0.71 |
| Interleukin-12 Subunit p40 (IL- | ng/mL | 0.15 | 0.051 | 0.12 | 0 | 0 | 25 | 0.0000 | 3.41 | 4.14 |
| 12p40) | | | | | | | | | | |
| Interleukin-15 (IL-15) | ng/mL | 0.35 | 0.20 | 0.29 | 0 | 2 | 68 | 0.0001 | 1.43 | 1.71 |
| Interleukin-16 (IL-16) | pg/mL | 208 | 62 | 202 | 52 | 393 | 126 | 0.0000 | 1.99 | 1.87 |
| Kallikrein 5 | ng/mL | 0.96 | 0.51 | 0.89 | 0 | 3 | 126 | 0.0000 | 3.64 | 3.60 |
| Kidney Injury Molecule-1 (KIM- | ng/mL | 0.16 | 0.34 | 0.089 | 0 | 3 | 126 | 0.0000 | 0.16 | 0.25 |
| 1) | | | | | | | | | | |
| Lactoylglutathione lyase (LGL) | ng/mL | 3.4 | 1.4 | 3.1 | 0 | 11 | 126 | 0.0000 | 5.04 | 4.21 |
| Latency-Associated Peptide of | ng/mL | 1.1 | 0.55 | 0.99 | 0 | 5 | 126 | 0.0000 | 9.75 | 11.14 |
| Transforming Growth Factor beta | | | | | | | | | | |
| 1 (LAP TGF-b1) | | | | | | | | | | |
| Lectin-Like Oxidized LDL | ng/mL | 0.60 | 0.13 | 0.53 | 0 | 1 | 15 | 0.0000 | 3.56 | 3.18 |
| Receptor 1 (LOX-1) | | | | | | | | | | |
| Leptin | ng/mL | 12 | 14 | 8.6 | 0 | 96 | 126 | 0.0005 | 1.67 | 1.98 |
| Macrophage Colony-Stimulating | ng/mL | 0.100 | 0.033 | 0.092 | 0 | 0 | 126 | 0.0000 | 5.02 | 4.90 |
| Factor 1 (M-CSF) | | | | | | | | | | |
| Macrophage-Derived Chemokine | pg/mL | 414 | 124 | 414 | 122 | 826 | 126 | 0.0007 | 1.22 | 1.23 |
| (MDC) | | | | | | | | | | |
| Macrophage inflammatory protein | pg/mL | 268 | 616 | 171 | 32 | 6760 | 126 | 0.0038 | 1.72 | 2.23 |
| 3 beta (MIP-3 beta) | | | | | | | | | | |
| Macrophage Migration Inhibitory | ng/mL | 0.056 | 0.046 | 0.044 | 0 | 0 | 123 | 0.0292 | 1.80 | 1.42 |
| Factor (MIF) | | | | | | | | | | |
| Macrophage-Stimulating Protein | ng/mL | 506 | 264 | 474 | 82 | 1350 | 126 | 0.0000 | 0.48 | 0.37 |
| (MSP) | | | | | | | | | | |
| Maspin | pg/mL | 1327 | 582 | 1160 | 863 | 3820 | 76 | 0.0000 | 1.99 | 2.11 |
| Matrix Metalloproteinase-1 | ng/mL | 1.2 | 0.97 | 0.95 | 0 | 10 | 117 | 0.0000 | 6.89 | 7.24 |
| (MMP-1) | | | | | | | | | | |
| Matrix Metalloproteinase-3 | ng/mL | 4.3 | 3.8 | 3.1 | 1 | 28 | 126 | 0.0000 | 2.47 | 2.95 |
| (MMP-3) | | | | | | | | | | |
| Matrix Metalloproteinase-9, total | ng/mL | 119 | 54 | 114 | 42 | 569 | 126 | 0.0000 | 5.51 | 5.18 |
| (MMP-9, total) | | | | | | | | | | |
| Mesothelin (MSLN) | nM | 63 | 31 | 56 | 12 | 252 | 126 | 0.0375 | 0.85 | 0.78 |
| Monocyte Chemotactic Protein 1 | pg/mL | 131 | 49 | 130 | 46 | 312 | 126 | 0.0000 | 3.44 | 3.15 |
| (MCP-1) | | | | | | | | | | |
| Monocyte Chemotactic Protein 2 | pg/mL | 33 | 24 | 28 | 9 | 234 | 126 | 0.0379 | 1.27 | 1.23 |
| (MCP-2) | | | | | | | | | | |
| Monocyte Chemotactic Protein 4 | pg/mL | 707 | 272 | 697 | 146 | 1660 | 126 | 0.0000 | 4.75 | 4.84 |
| (MCP-4) | | | | | | | | | | |
| Myeloid Progenitor Inhibitory | ng/mL | 0.80 | 0.50 | 0.67 | 0 | 4 | 126 | 0.0000 | 2.14 | 2.23 |
| Factor 1 (MPIF-1) | | | | | | | | | | |
| Myeloperoxidase (MPO) | ng/mL | 199 | 139 | 158 | 46 | 716 | 124 | 0.0000 | 5.24 | 4.69 |
| Myoglobin | ng/mL | 15 | 12 | 11 | 4 | 89 | 126 | 0.0000 | 10.16 | 8.81 |
| Neuron-Specific Enolase (NSE) | ng/mL | 14 | 11 | 10 | 2 | 84 | 112 | 0.0000 | 0.18 | 0.11 |
| Neuronal Cell Adhesion Molecule | ng/mL | 1.6 | 0.89 | 1.5 | 0 | 6 | 119 | 0.0000 | 0.52 | 0.50 |
| (Nr-CAM) | | | | | | | | | | |
| Neuropilin-1 | ng/mL | 167 | 36 | 161 | 103 | 319 | 126 | 0.0000 | 0.84 | 0.88 |
| Neutrophil Gelatinase-Associated | ng/mL | 106 | 48 | 88 | 40 | 263 | 102 | 0.0000 | 2.27 | 2.61 |
| Lipocalin (NGAL) | | | | | | | | | | |
| Osteopontin | ng/mL | 5.1 | 6.8 | 3.6 | 1 | 66 | 126 | 0.0131 | 1.41 | 1.72 |
| Osteoprotegerin (OPG) | pM | 4.6 | 1.4 | 4.2 | 2 | 8 | 126 | 0.0328 | 1.11 | 1.13 |
| Plasminogen Activator Inhibitor 1 | ng/mL | 32 | 29 | 23 | 4 | 166 | 126 | 0.0000 | 6.12 | 8.16 |
| (PAI-1) | | | | | | | | | | |
| Platelet-Derived Growth Factor | pg/mL | 271 | 327 | 167 | 42 | 2390 | 79 | 0.0000 | 57.62 | 93.41 |
| BB (PDGF-BB) | | | | | | | | | | |
| Progesterone | ng/mL | 6.0 | 4.2 | 4.8 | 1 | 30 | 119 | 0.0000 | 2.00 | 2.10 |
| Proinsulin, Intact | pM | 5.8 | 3.6 | 5.5 | 2 | 22 | 43 | 0.0010 | 3.11 | 3.10 |
| Proinsulin, Total | pM | 15 | 15 | 11 | 4 | 93 | 68 | 0.0046 | 3.61 | 5.14 |
| Protein S100-A4 (S100-A4) | ng/mL | 6.6 | 7.8 | 4.6 | 0 | 63 | 102 | 0.0183 | 7.73 | 8.44 |
| Pulmonary and Activation- | ng/mL | 91 | 39 | 84 | 27 | 227 | 126 | 0.0249 | 0.83 | 0.76 |
| Regulated Chemokine (PARC) | | | | | | | | | | |
| Receptor for advanced | ng/mL | 2.5 | 1.7 | 2.0 | 0 | 7 | 125 | 0.0000 | 1.91 | 2.03 |
| glycosylation end products | | | | | | | | | | |
| (RAGE) | | | | | | | | | | |
| Receptor tyrosine-protein kinase | ng/mL | 2.2 | 0.85 | 2.3 | 0 | 4 | 126 | 0.0000 | 0.29 | 0.14 |
| erbB-3 (ErbB3) | | | | | | | | | | |
| Resistin | ng/mL | 2.7 | 1.1 | 2.3 | 1 | 7 | 126 | 0.0000 | 0.67 | 0.65 |
| Serotransferrin (Transferrin) | mg/dl | 327 | 90 | 305 | 188 | 657 | 126 | 0.0000 | 0.73 | 0.76 |
| Serum Amyloid P-Component | ug/mL | 25 | 8.0 | 24 | 9 | 50 | 126 | 0.0000 | 0.69 | 0.71 |
| (SAP) | | | | | | | | | | |
| Sortilin | ng/mL | 5.1 | 1.5 | 4.9 | 1 | 9 | 126 | 0.0000 | 1.47 | 1.48 |
| Squamous Cell Carcinoma | ng/mL | 0.50 | 0.65 | 0.36 | 0 | 6 | 125 | 0.0000 | 3.05 | 4.47 |
| Antigen-1 (SCCA-1) | | | | | | | | | | |
| Stem Cell Factor (SCF) | pg/mL | 212 | 92 | 199 | 24 | 879 | 126 | 0.0000 | 1.55 | 1.55 |
| Stromal cell-derived factor-1 | pg/mL | 1614 | 523 | 1670 | 516 | 3730 | 126 | 0.0000 | 2.29 | 2.26 |
| (SDF-1) | | | | | | | | | | |
| Superoxide Dismutase 1, soluble | ng/mL | 7.5 | 4.7 | 6.2 | 3 | 43 | 126 | 0.0000 | 3.19 | 2.72 |
| (SOD-1) | | | | | | | | | | |
| T-Cell-Specific Protein RANTES | ng/mL | 1.4 | 1.7 | 1.1 | 0 | 14 | 126 | 0.0000 | 12.69 | 17.14 |
| (RANTES) | | | | | | | | | | |
| Tenascin-C (TN-C) | ng/mL | 466 | 241 | 423 | 184 | 2160 | 126 | 0.0130 | 0.82 | 0.80 |
| Tetranectin | ug/mL | 14 | 2.8 | 14 | 8 | 21 | 126 | 0.0171 | 1.10 | 1.05 |
| Thrombomodulin (TM) | ng/mL | 5.1 | 1.4 | 4.8 | 2 | 9 | 126 | 0.0000 | 0.81 | 0.84 |
| Thrombospondin-1 | ng/mL | 876 | 1160 | 584 | 44 | 8460 | 125 | 0.0000 | 11.19 | 16.34 |
| Thyroxine-Binding Globulin | ug/mL | 65 | 18 | 63 | 27 | 131 | 126 | 0.0000 | 0.57 | 0.61 |
| (TBG) | | | | | | | | | | |
| Tissue Inhibitor of | ng/mL | 71 | 19 | 70 | 35 | 136 | 126 | 0.0000 | 1.94 | 2.01 |
| Metalloproteinases 1 (TIMP-1) | | | | | | | | | | |
| Tissue type Plasminogen activator | ng/mL | 0.93 | 0.47 | 0.81 | 0 | 2 | 126 | 0.0000 | 1.60 | 1.61 |
| (tPA) | | | | | | | | | | |
| Transthyretin (TTR) | mg/dl | 33 | 13 | 32 | 9 | 69 | 126 | 0.0000 | 0.67 | 0.66 |
| Trefoil Factor 3 (TFF3) | ug/mL | 0.064 | 0.030 | 0.058 | 0 | 0 | 125 | 0.0377 | 3.67 | 1.42 |
| Tumor Necrosis Factor alpha | pg/mL | 6.0 | 6.3 | 3.8 | 2 | 37 | 62 | 0.0304 | 5.47 | 9.66 |
| (TNF-alpha) | | | | | | | | | | |
| Tumor Necrosis Factor Receptor I | pg/mL | 1246 | 394 | 1215 | 518 | 3190 | 126 | 0.0096 | 1.16 | 1.23 |
| (TNF RI) | | | | | | | | | | |
| Tumor necrosis factor receptor 2 | ng/mL | 5.5 | 1.6 | 5.3 | 2 | 12 | 126 | 0.0290 | 0.91 | 0.92 |
| (TNFR2) | | | | | | | | | | |
| Urokinase-type plasminogen | ng/mL | 1.5 | 0.84 | 1.3 | 0 | 6 | 125 | 0.0065 | 0.81 | 0.85 |
| activator receptor (uPAR) | | | | | | | | | | |
| Vascular Cell Adhesion | ng/mL | 612 | 214 | 561 | 303 | 1510 | 126 | 0.0094 | 0.89 | 0.92 |
| Molecule-1 (VCAM-1) | | | | | | | | | | |
| Vascular Endothelial Growth | ng/mL | 3.9 | 1.0 | 3.8 | 2 | 9 | 126 | 0.0000 | 9.07 | 9.60 |
| Factor C (VEGF-C) | | | | | | | | | | |
| Vascular Endothelial Growth | ng/mL | 5.8 | 1.2 | 5.8 | 2 | 9 | 126 | 0.0000 | 1.23 | 1.22 |
| Factor Receptor 2 (VEGFR-2) | | | | | | | | | | |
| Vascular endothelial growth | ng/mL | 44 | 19 | 42 | 8 | 92 | 126 | 0.0004 | 1.35 | 1.41 |
| factor receptor 3 (VEGFR-3) | | | | | | | | | | |
| Vitamin K-Dependent Protein S | ug/mL | 23 | 5.8 | 22 | 14 | 54 | 126 | 0.0000 | 0.65 | 0.69 |
| (VKDPS) | | | | | | | | | | |
| Vitronectin | ug/mL | 1791 | 420 | 1780 | 745 | 2870 | 126 | 0.0000 | 1.34 | 1.33 |
| von Willebrand Factor (vWF) | ug/mL | 40 | 20 | 37 | 9 | 117 | 126 | 0.0010 | 1.32 | 1.31 |
| YKL-40 | ng/mL | 35 | 22 | 28 | 15 | 173 | 126 | 0.0105 | 0.79 | 0.89 |

TABLE 10b
Phase II Clinical Trial - 140 analytes (biomarkers) which had a p value of <0.05 (47 HIBM samples)

| | | | | | | | | | change in | change in |
|---|---|---|---|---|---|---|---|---|---|---|
| | | | | | | | | | mean | median |
| | | HIBM | HIBM | HIBM | | | Positive | p- | (HIBM/ | (HIBM/ |
| 140 markers with p < 0.05. | Units | Mean | SD | Median | Min | Max | Count | value | nomral) | normal) |
| 6Ckine | pg/mL | 456.49 | 82.91 | 460.00 | 260.00 | 664.00 | 47 | 0.0001 | 0.85 | 0.89 |
| Aldose Reductase | ng/mL | 11.67 | 5.01 | 9.90 | 5.60 | 27.00 | 47 | 0.0000 | 3.45 | 2.94 |
| Alpha-1-Microglobulin | ug/mL | 10.42 | 2.20 | 10.00 | 6.50 | 16.00 | 47 | 0.0467 | 0.93 | 0.92 |
| (A1Micro) | | | | | | | | | | |
| Alpha-2-Macroglobulin | mg/mL | 1.54 | 0.34 | 1.50 | 0.92 | 2.60 | 47 | 0.0000 | 0.47 | 0.52 |
| (A2Macro) | | | | | | | | | | |
| Angiogenin | ng/mL | 413.00 | 136.72 | 379.00 | 192.00 | 903.00 | 47 | 0.0000 | 0.66 | 0.64 |
| Angiopoietin-2 (ANG-2) | ng/mL | 4.20 | 1.23 | 4.10 | 1.90 | 7.70 | 47 | 0.0000 | 1.32 | 1.38 |
| Angiotensin-Converting | ng/mL | 64.83 | 16.95 | 61.00 | 37.00 | 105.00 | 47 | 0.0000 | 0.71 | 0.70 |
| Enzyme (ACE) | | | | | | | | | | |
| Apolipoprotein(a) (Lp(a)) | ug/mL | 305.24 | 314.28 | 226.00 | 5.40 | 1740.00 | 47 | 0.0000 | 0.39 | 0.45 |
| Apolipoprotein A-I (Apo A-I) | mg/mL | 1.62 | 0.29 | 1.60 | 1.10 | 2.40 | 47 | 0.0000 | 1.25 | 1.25 |
| Apolipoprotein A-II (Apo A-II) | ng/mL | 331.30 | 57.00 | 322.00 | 253.00 | 536.00 | 47 | 0.0000 | 0.62 | 0.72 |
| Apolipoprotein A-IV (Apo A- | ug/mL | 16.51 | 20.08 | 11.00 | 6.30 | 127.00 | 47 | 0.0000 | 0.44 | 0.33 |
| IV) | | | | | | | | | | |
| Apolipoprotein B (Apo B) | ug/mL | 1304.43 | 405.44 | 1390.00 | 387.00 | 2090.00 | 47 | 0.0000 | 0.65 | 0.76 |
| Apolipoprotein C-III (Apo C- | ug/mL | 233.91 | 86.35 | 205.00 | 141.00 | 538.00 | 47 | 0.0000 | 1.43 | 1.52 |
| III) | | | | | | | | | | |
| Apolipoprotein D (Apo D) | ug/mL | 124.73 | 32.04 | 122.00 | 75.00 | 199.00 | 40 | 0.0000 | 0.65 | 0.69 |
| Apolipoprotein H (Apo H) | ug/mL | 249.57 | 50.16 | 248.00 | 137.00 | 388.00 | 47 | 0.0000 | 0.84 | 0.88 |
| AXL Receptor Tyrosine Kinase | ng/mL | 11.38 | 3.35 | 11.00 | 5.20 | 21.00 | 47 | 0.0044 | 1.17 | 1.15 |
| (AXL) | | | | | | | | | | |
| B cell-activating factor (BAFF) | pg/mL | 834.85 | 165.90 | 831.00 | 486.00 | 1190.00 | 47 | 0.0000 | 1.22 | 1.27 |
| B Lymphocyte | pg/mL | 70.50 | 4.50 | 70.50 | 66.00 | 75.00 | 2 | 0.0006 | 3.63 | 8.17 |
| Chemoattractant (BLC) | | | | | | | | | | |
| Beta-2-Microglobulin (B2M) | ug/mL | 1.31 | 0.27 | 1.30 | 0.88 | 1.90 | 47 | 0.0000 | 0.77 | 0.80 |
| Brain-Derived Neurotrophic | ng/mL | 19.72 | 4.80 | 20.00 | 8.80 | 31.00 | 47 | 0.0000 | 34.58 | 50.06 |
| Factor (BDNF) | | | | | | | | | | |
| C-Peptide | ng/mL | 2.49 | 1.32 | 2.30 | 0.73 | 6.10 | 47 | 0.0000 | 1.77 | 2.14 |
| Carcinoembryonic Antigen | ng/mL | 0.98 | 0.55 | 0.82 | 0.36 | 3.40 | 47 | 0.0000 | 0.46 | 0.52 |
| (CEA) | | | | | | | | | | |
| Cathepsin D | ng/mL | 288.72 | 100.80 | 281.00 | 136.00 | 610.00 | 47 | 0.0001 | 0.81 | 0.81 |
| CD5 Antigen-like (CD5L) | ng/mL | 2530.21 | 1024.10 | 2470.00 | 1150.00 | 6160.00 | 47 | 0.0000 | 1.56 | 1.70 |
| CD 40 antigen (CD40) | ng/mL | 0.92 | 0.32 | 0.89 | 0.48 | 2.60 | 47 | 0.0000 | 2.10 | 2.13 |
| CD40 Ligand (CD40-L) | ng/mL | 1.75 | 0.90 | 1.60 | 0.12 | 3.90 | 47 | 0.0000 | 57.13 | 82.90 |
| Cellular Fibronectin (cFib) | ug/mL | 2.14 | 0.90 | 1.85 | 0.89 | 4.30 | 40 | 0.0000 | 3.23 | 2.92 |
| Chromogranin-A (CgA) | ng/mL | 344.73 | 164.73 | 327.00 | 30.00 | 871.00 | 41 | 0.0000 | 4.36 | 25.95 |
| Clusterin (CLU) | ug/mL | 257.40 | 29.27 | 259.00 | 210.00 | 330.00 | 47 | 0.0000 | 0.85 | 0.91 |
| Complement C3 (C3) | mg/mL | 1.25 | 0.23 | 1.20 | 0.79 | 1.90 | 47 | 0.0000 | 0.57 | 0.57 |
| Complement Factor H - | ug/mL | 2328.70 | 793.31 | 2290.00 | 879.00 | 3900.00 | 47 | 0.0000 | 0.70 | 0.69 |
| Related Protein 1 (CFHR1) | | | | | | | | | | |
| Cortisol (Cortisol) | ng/mL | 223.43 | 87.78 | 207.00 | 120.00 | 643.00 | 47 | 0.0000 | 2.21 | 2.20 |
| Creatine Kinase-MB (CK-MB) | ng/mL | 12.36 | 10.04 | 8.70 | 3.30 | 47.00 | 47 | 0.0000 | 11.37 | 10.90 |
| Cystatin-C | ng/mL | 812.17 | 125.92 | 817.00 | 521.00 | 1190.00 | 47 | 0.0000 | 0.78 | 0.83 |
| E-Selectin | ng/mL | 8.87 | 4.33 | 8.20 | 0.23 | 23.00 | 47 | 0.0000 | 0.46 | 0.43 |
| EN-RAGE | ng/mL | 43.93 | 49.68 | 26.00 | 7.00 | 291.00 | 47 | 0.0003 | 2.90 | 2.28 |
| Endoglin | ng/mL | 3.31 | 0.67 | 3.40 | 2.00 | 4.60 | 47 | 0.0000 | 0.84 | 0.90 |
| Endostatin | ng/mL | 96.66 | 29.09 | 97.00 | 40.00 | 172.00 | 47 | 0.0003 | 1.23 | 1.26 |
| Eotaxin-1 | pg/mL | 207.05 | 39.57 | 209.00 | 148.00 | 299.00 | 21 | 0.0000 | 4.90 | 7.39 |
| Epidermal Growth Factor | pg/mL | 278.28 | 157.61 | 248.00 | 44.00 | 656.00 | 46 | 0.0000 | 23.55 | 53.22 |
| (EGF) | | | | | | | | | | |
| Epidermal Growth Factor | ng/mL | 4.03 | 0.52 | 4.10 | 2.90 | 4.80 | 47 | 0.0008 | 0.92 | 0.94 |
| Receptor (EGFR) | | | | | | | | | | |
| Epithelial-Derived Neutrophil- | ng/mL | 2.46 | 1.34 | 2.00 | 0.67 | 6.40 | 47 | 0.0000 | 6.42 | 6.85 |
| Activating Protein 78 (ENA- | | | | | | | | | | |
| 78) | | | | | | | | | | |
| Factor VII | ng/mL | 391.04 | 136.04 | 367.00 | 127.00 | 722.00 | 47 | 0.0016 | 1.26 | 1.21 |
| FASLG Receptor (FAS) | ng/mL | 16.23 | 8.02 | 15.00 | 6.30 | 56.00 | 45 | 0.0007 | 1.55 | 1.88 |
| Fatty Acid-Binding Protein, | ng/mL | 20.78 | 9.05 | 19.00 | 2.50 | 50.00 | 47 | 0.0001 | 1.50 | 1.60 |
| adipocyte (FABP, adipocyte) | | | | | | | | | | |
| Fetuin-A | ug/mL | 742.28 | 108.37 | 738.00 | 473.00 | 989.00 | 47 | 0.0000 | 0.58 | 0.62 |
| Fibrinogen | mg/mL | 0.07 | 0.01 | 0.06 | 0.06 | 0.10 | 8 | 0.0000 | 2.48 | 2.51 |
| Galectin-3 | ng/mL | 13.41 | 2.68 | 13.00 | 8.30 | 19.00 | 47 | 0.0000 | 1.26 | 1.25 |
| Gelsolin | ug/mL | 50.83 | 16.58 | 50.00 | 22.00 | 96.00 | 47 | 0.0000 | 0.77 | 0.77 |
| Glucagon-like Peptide 1, total | pg/mL | 16.99 | 13.62 | 11.50 | 7.60 | 60.00 | 16 | 0.0011 | 6.09 | 4.71 |
| (GLP-1 total) | | | | | | | | | | |
| Glutathione S-Transferase Mu | ng/mL | 6.50 | 0.00 | 6.50 | 6.50 | 6.50 | 5 | 0.0000 | 1.38 | 1.53 |
| 1 (GST-M1) | | | | | | | | | | |
| Granulocyte Colony- | pg/mL | 10.00 | 3.11 | 8.80 | 6.30 | 20.00 | 44 | 0.0433 | 1.16 | 1.20 |
| Stimulating Factor (G-CSF) | | | | | | | | | | |
| Hepatocyte Growth Factor | ng/mL | 14.18 | 8.41 | 13.00 | 2.80 | 35.00 | 47 | 0.0000 | 4.59 | 5.04 |
| (HGF) | | | | | | | | | | |
| Hepsin | pg/mL | 1074.19 | 169.08 | 1050.00 | 728.00 | 1390.00 | 47 | 0.0000 | 1.32 | 1.28 |
| Human Epidermal Growth | ng/mL | 0.47 | 0.16 | 0.45 | 0.17 | 0.88 | 47 | 0.0002 | 0.81 | 0.80 |
| Factor Receptor 2 (HER-2) | | | | | | | | | | |
| Immunoglobulin A (IgA) | mg/mL | 2.14 | 0.86 | 1.95 | 0.56 | 4.20 | 46 | 0.0000 | 0.57 | 0.58 |
| Immunoglobulin M (IgM) | mg/mL | 1.84 | 1.17 | 1.50 | 0.53 | 7.00 | 47 | 0.0000 | 0.65 | 0.57 |
| Insulin-like Growth Factor- | ng/mL | 51.98 | 28.71 | 42.00 | 18.00 | 169.00 | 47 | 0.0000 | 0.65 | 0.60 |
| Binding Protein 2 (IGFBP-2) | | | | | | | | | | |
| Insulin-like Growth Factor | ng/mL | 364.77 | 74.83 | 362.00 | 230.00 | 556.00 | 47 | 0.0000 | 0.70 | 0.71 |
| Binding Protein 4 (IGFBP4) | | | | | | | | | | |
| Insulin-like Growth Factor | ng/mL | 129.87 | 28.79 | 132.00 | 75.00 | 205.00 | 47 | 0.0006 | 0.88 | 0.91 |
| Binding Protein 5 (IGFBP5) | | | | | | | | | | |
| Insulin-like Growth Factor | ng/mL | 364.96 | 146.83 | 365.00 | 69.00 | 880.00 | 47 | 0.0000 | 2.90 | 3.49 |
| Binding Protein 6 (IGFBP6) | | | | | | | | | | |
| Intercellular Adhesion | ng/mL | 123.66 | 34.06 | 122.00 | 50.00 | 281.00 | 47 | 0.0002 | 1.26 | 1.25 |
| Molecule 1 (ICAM-1) | | | | | | | | | | |
| Interferon gamma Induced | pg/mL | 455.23 | 333.07 | 349.00 | 149.00 | 2150.00 | 47 | 0.0101 | 1.45 | 1.28 |
| Protein 10 (IP-10) | | | | | | | | | | |
| Interferon-inducible T-cell | pg/mL | 59.24 | 63.01 | 42.00 | 26.00 | 435.00 | 42 | 0.0001 | 3.88 | 3.82 |
| alpha chemoattractant (ITAC) | | | | | | | | | | |
| Interleukin-1 alpha (IL-1 alpha) | ng/mL | 0.00 | 0.00 | 0.00 | 0.00 | 0.01 | 40 | 0.0000 | 1.99 | 2.23 |
| Interleukin-1 receptor | pg/mL | 173.81 | 49.70 | 159.00 | 97.00 | 313.00 | 42 | 0.0000 | 2.87 | 3.11 |
| antagonist (IL-1ra) | | | | | | | | | | |
| Interleukin-2 receptor alpha | pg/mL | 2363.19 | 639.55 | 2380.00 | 1020.00 | 4180.00 | 47 | 0.0000 | 1.64 | 1.71 |
| (IL-2 receptor alpha) | | | | | | | | | | |
| Interleukin-6 receptor (IL-6r) | ng/mL | 29.60 | 8.49 | 28.00 | 13.00 | 53.00 | 47 | 0.0000 | 1.30 | 1.34 |
| Interleukin-6 receptor subunit | ng/mL | 164.81 | 30.28 | 163.00 | 94.00 | 241.00 | 47 | 0.0003 | 0.89 | 0.88 |
| beta (IL-6R beta) | | | | | | | | | | |
| Interleukin-7 (IL-7) | pg/mL | 12.50 | 5.16 | 10.00 | 10.00 | 24.00 | 6 | 0.0463 | 0.59 | 0.71 |
| Interleukin-12 Subunit p40 (IL- | ng/mL | 0.50 | 0.14 | 0.48 | 0.30 | 0.91 | 36 | 0.0000 | 3.41 | 4.14 |
| 12p40) | | | | | | | | | | |
| Interleukin-15 (IL-15) | ng/mL | 0.50 | 0.08 | 0.49 | 0.42 | 0.70 | 12 | 0.0001 | 1.43 | 1.71 |
| Interleukin-16 (IL-16) | pg/mL | 415.51 | 165.09 | 377.00 | 157.00 | 1050.00 | 47 | 0.0000 | 1.99 | 1.87 |
| Kallikrein 5 | ng/mL | 3.50 | 1.63 | 3.20 | 0.84 | 8.80 | 47 | 0.0000 | 3.64 | 3.60 |
| Kidney Injury Molecule-1 | ng/mL | 0.02 | 0.01 | 0.02 | 0.02 | 0.05 | 14 | 0.0000 | 0.16 | 0.25 |
| (KIM-1) | | | | | | | | | | |
| Lactoylglutathione lyase (LGL) | ng/mL | 17.26 | 16.54 | 13.00 | 2.80 | 102.00 | 47 | 0.0000 | 5.04 | 4.21 |
| Latency-Associated Peptide of | ng/mL | 10.63 | 3.05 | 11.00 | 5.00 | 19.00 | 47 | 0.0000 | 9.75 | 11.14 |
| Transforming Growth Factor | | | | | | | | | | |
| beta 1 (LAP TGF-b1) | | | | | | | | | | |
| Lectin-Like Oxidized LDL | ng/mL | 2.12 | 1.04 | 1.70 | 0.93 | 4.70 | 32 | 0.0000 | 3.56 | 3.18 |
| Receptor 1 (LOX-1) | | | | | | | | | | |
| Leptin | ng/mL | 20.51 | 12.97 | 17.00 | 2.60 | 52.00 | 47 | 0.0005 | 1.67 | 1.98 |
| Macrophage Colony- | ng/mL | 0.50 | 0.16 | 0.45 | 0.26 | 0.96 | 45 | 0.0000 | 5.02 | 4.90 |
| Stimulating Factor 1 (M-CSF) | | | | | | | | | | |
| Macrophage-Derived | pg/mL | 505.45 | 157.29 | 507.00 | 111.00 | 1110.00 | 47 | 0.0007 | 1.22 | 1.23 |
| Chemokine (MDC) | | | | | | | | | | |
| Macrophage inflammatory | pg/mL | 460.60 | 242.36 | 382.00 | 209.00 | 1620.00 | 47 | 0.0038 | 1.72 | 2.23 |
| protein 3 beta (MIP-3 beta) | | | | | | | | | | |
| Macrophage Migration | ng/mL | 0.10 | 0.12 | 0.06 | 0.01 | 0.57 | 42 | 0.0292 | 1.80 | 1.42 |
| Inhibitory Factor (MIF) | | | | | | | | | | |
| Macrophage-Stimulating | ng/mL | 240.23 | 163.74 | 177.00 | 63.00 | 800.00 | 47 | 0.0000 | 0.48 | 0.37 |
| Protein (MSP) | | | | | | | | | | |
| Maspin | pg/mL | 2640.00 | 630.17 | 2450.00 | 1970.00 | 4520.00 | 11 | 0.0000 | 1.99 | 2.11 |
| Matrix Metalloproteinase-1 | ng/mL | 7.93 | 4.92 | 6.90 | 1.10 | 21.00 | 47 | 0.0000 | 6.89 | 7.24 |
| (MMP-1) | | | | | | | | | | |
| Matrix Metalloproteinase-3 | ng/mL | 10.50 | 5.50 | 9.00 | 2.60 | 25.00 | 47 | 0.0000 | 2.47 | 2.95 |
| (MMP-3) | | | | | | | | | | |
| Matrix Metalloproteinase-9, | ng/mL | 657.09 | 197.30 | 591.00 | 326.00 | 1310.00 | 47 | 0.0000 | 5.51 | 5.18 |
| total (MMP-9, total) | | | | | | | | | | |
| Mesothelin (MSLN) | nM | 53.38 | 23.82 | 44.00 | 21.00 | 119.00 | 47 | 0.0375 | 0.85 | 0.78 |
| Monocyte Chemotactic Protein | pg/mL | 450.26 | 176.19 | 409.00 | 215.00 | 1220.00 | 47 | 0.0000 | 3.44 | 3.15 |
| 1 (MCP-1) | | | | | | | | | | |
| Monocyte Chemotactic Protein | pg/mL | 41.81 | 25.01 | 35.00 | 14.00 | 148.00 | 47 | 0.0379 | 1.27 | 1.23 |
| 2 (MCP-2) | | | | | | | | | | |
| Monocyte Chemotactic Protein | pg/mL | 3361.28 | 1297.41 | 3370.00 | 1140.00 | 7310.00 | 47 | 0.0000 | 4.75 | 4.84 |
| 4 (MCP-4) | | | | | | | | | | |
| Myeloid Progenitor Inhibitory | ng/mL | 1.72 | 1.09 | 1.50 | 0.69 | 7.40 | 47 | 0.0000 | 2.14 | 2.23 |
| Factor 1 (MPIF-1) | | | | | | | | | | |
| Myeloperoxidase (MPO) | ng/mL | 1045.60 | 901.05 | 741.00 | 193.00 | 4380.00 | 47 | 0.0000 | 5.24 | 4.69 |
| Myoglobin | ng/mL | 152.09 | 112.88 | 100.00 | 47.00 | 551.00 | 47 | 0.0000 | 10.16 | 8.81 |
| Neuron-Specific Enolase | ng/mL | 2.51 | 6.41 | 1.10 | 0.32 | 45.00 | 47 | 0.0000 | 0.18 | 0.11 |
| (NSE) | | | | | | | | | | |
| Neuronal Cell Adhesion | ng/mL | 0.84 | 0.37 | 0.72 | 0.22 | 1.80 | 47 | 0.0000 | 0.52 | 0.50 |
| Molecule (Nr-CAM) | | | | | | | | | | |
| Neuropilin-1 | ng/mL | 140.19 | 30.15 | 142.00 | 90.00 | 229.00 | 47 | 0.0000 | 0.84 | 0.88 |
| Neutrophil Gelatinase- | ng/mL | 240.57 | 82.96 | 229.00 | 87.00 | 494.00 | 47 | 0.0000 | 2.27 | 2.61 |
| Associated Lipocalin (NGAL) | | | | | | | | | | |
| Osteopontin | ng/mL | 7.22 | 3.25 | 6.20 | 3.30 | 16.00 | 33 | 0.0131 | 1.41 | 1.72 |
| Osteoprotegerin (OPG) | pM | 5.08 | 1.37 | 4.70 | 3.30 | 11.00 | 47 | 0.0328 | 1.11 | 1.13 |
| Plasminogen Activator | ng/mL | 196.30 | 56.14 | 191.00 | 88.00 | 316.00 | 47 | 0.0000 | 6.12 | 8.16 |
| Inhibitor 1 (PAI-1) | | | | | | | | | | |
| Platelet-Derived Growth Factor | pg/mL | 15631.91 | 4560.00 | 15600.00 | 7260.00 | 24600.00 | 47 | 0.0000 | 57.62 | 93.41 |
| BB (PDGF-BB) | | | | | | | | | | |
| Progesterone | ng/mL | 12.13 | 5.63 | 10.00 | 6.50 | 28.00 | 38 | 0.0000 | 2.00 | 2.10 |
| Proinsulin, Intact | pM | 18.00 | 2.35 | 17.00 | 16.00 | 22.00 | 4 | 0.0010 | 3.11 | 3.10 |
| Proinsulin, Total | pM | 54.75 | 10.33 | 55.00 | 43.00 | 66.00 | 4 | 0.0046 | 3.61 | 5.14 |
| Protein S100-A4 (S100-A4) | ng/mL | 50.87 | 46.18 | 38.50 | 9.70 | 149.00 | 10 | 0.0183 | 7.73 | 8.44 |
| Pulmonary and Activation- | ng/mL | 75.30 | 39.78 | 64.00 | 24.00 | 201.00 | 47 | 0.0249 | 0.83 | 0.76 |
| Regulated Chemokine (PARC) | | | | | | | | | | |
| Receptor for advanced | ng/mL | 4.77 | 3.05 | 4.00 | 1.00 | 16.00 | 47 | 0.0000 | 1.91 | 2.03 |
| glycosylation end products | | | | | | | | | | |
| (RAGE) | | | | | | | | | | |
| Receptor tyrosine-protein | ng/mL | 0.62 | 0.69 | 0.31 | 0.18 | 3.30 | 47 | 0.0000 | 0.29 | 0.14 |
| kinase erbB-3 (ErbB3) | | | | | | | | | | |
| Resistin | ng/mL | 1.78 | 0.81 | 1.50 | 0.44 | 5.50 | 47 | 0.0000 | 0.67 | 0.65 |
| Serotransferrin (Transferrin) | mg/dl | 238.47 | 35.67 | 230.00 | 178.00 | 370.00 | 47 | 0.0000 | 0.73 | 0.76 |
| Serum Amyloid P-Component | ug/mL | 17.26 | 5.01 | 17.00 | 8.00 | 32.00 | 47 | 0.0000 | 0.69 | 0.71 |
| (SAP) | | | | | | | | | | |
| Sortilin | ng/mL | 7.57 | 1.83 | 7.20 | 4.40 | 12.00 | 47 | 0.0000 | 1.47 | 1.48 |
| Squamous Cell Carcinoma | ng/mL | 1.53 | 0.19 | 1.60 | 1.20 | 1.80 | 7 | 0.0000 | 3.05 | 4.47 |
| Antigen-1 (SCCA-1) | | | | | | | | | | |
| Stem Cell Factor (SCF) | pg/mL | 328.40 | 112.43 | 308.00 | 164.00 | 620.00 | 47 | 0.0000 | 1.55 | 1.55 |
| Stromal cell-derived factor-1 | pg/mL | 3703.28 | 677.81 | 3780.00 | 454.00 | 4790.00 | 47 | 0.0000 | 2.29 | 2.26 |
| (SDF-1) | | | | | | | | | | |
| Superoxide Dismutase 1, | ng/mL | 23.93 | 18.88 | 17.00 | 8.60 | 91.00 | 47 | 0.0000 | 3.19 | 2.72 |
| soluble (SOD-1) | | | | | | | | | | |
| T-Cell-Specific Protein | ng/mL | 18.26 | 7.29 | 18.00 | 3.70 | 36.00 | 47 | 0.0000 | 12.69 | 17.14 |
| RANTES (RANTES) | | | | | | | | | | |
| Tenascin-C (TN-C) | ng/mL | 381.17 | 174.87 | 340.00 | 87.00 | 1100.00 | 47 | 0.0130 | 0.82 | 0.80 |
| Tetranectin | ug/mL | 15.92 | 3.68 | 15.00 | 9.10 | 23.00 | 47 | 0.0171 | 1.10 | 1.05 |
| Thrombomodulin (TM) | ng/mL | 4.10 | 0.93 | 4.00 | 2.30 | 7.10 | 47 | 0.0000 | 0.81 | 0.84 |
| Thrombospondin-1 | ng/mL | 9800.64 | 2558.16 | 9540.00 | 3930.00 | 14700.00 | 47 | 0.0000 | 11.19 | 16.34 |
| Thyroxine-Binding Globulin | ug/mL | 37.36 | 7.45 | 38.00 | 22.00 | 55.00 | 47 | 0.0000 | 0.57 | 0.61 |
| (TBG) | | | | | | | | | | |
| Tissue Inhibitor of | ng/mL | 138.49 | 23.17 | 140.00 | 99.00 | 189.00 | 47 | 0.0000 | 1.94 | 2.01 |
| Metalloproteinases 1 (TIMP-1) | | | | | | | | | | |
| Tissue type Plasminogen | ng/mL | 1.49 | 0.73 | 1.30 | 0.46 | 3.30 | 47 | 0.0000 | 1.60 | 1.61 |
| activator (tPA) | | | | | | | | | | |
| Transthyretin (TTR) | mg/dl | 22.04 | 3.91 | 21.00 | 15.00 | 33.00 | 47 | 0.0000 | 0.67 | 0.66 |
| Trefoil Factor 3 (TFF3) | ug/mL | 0.23 | 0.54 | 0.08 | 0.04 | 3.50 | 47 | 0.0377 | 3.67 | 1.42 |
| Tumor Necrosis Factor alpha | pg/mL | 33.00 | 7.12 | 37.00 | 23.00 | 39.00 | 3 | 0.0304 | 5.47 | 9.66 |
| (TNF-alpha) | | | | | | | | | | |
| Tumor Necrosis Factor | pg/mL | 1448.77 | 459.65 | 1500.00 | 162.00 | 2480.00 | 47 | 0.0096 | 1.16 | 1.23 |
| Receptor I (TNF RI) | | | | | | | | | | |
| Tumor necrosis factor receptor | ng/mL | 5.04 | 1.18 | 4.90 | 2.90 | 8.30 | 47 | 0.0290 | 0.91 | 0.92 |
| 2 (TNFR2) | | | | | | | | | | |
| Urokinase-type plasminogen | ng/mL | 1.21 | 0.50 | 1.10 | 0.35 | 2.90 | 47 | 0.0065 | 0.81 | 0.85 |
| activator receptor (uPAR) | | | | | | | | | | |
| Vascular Cell Adhesion | ng/mL | 545.23 | 113.03 | 518.00 | 338.00 | 862.00 | 47 | 0.0094 | 0.89 | 0.92 |
| Molecule-1 (VCAM-1) | | | | | | | | | | |
| Vascular Endothelial Growth | ng/mL | 35.32 | 6.73 | 36.00 | 22.00 | 46.00 | 47 | 0.0000 | 9.07 | 9.60 |
| Factor C (VEGF-C) | | | | | | | | | | |
| Vascular Endothelial Growth | ng/mL | 7.09 | 1.30 | 7.00 | 3.50 | 9.70 | 47 | 0.0000 | 1.23 | 1.22 |
| Factor Receptor 2 (VEGFR-2) | | | | | | | | | | |
| Vascular endothelial growth | ng/mL | 60.15 | 26.03 | 59.00 | 13.00 | 120.00 | 47 | 0.0004 | 1.35 | 1.41 |
| factor receptor 3 (VEGFR-3) | | | | | | | | | | |
| Vitamin K-Dependent Protein | ug/mL | 15.06 | 3.27 | 15.00 | 8.00 | 26.00 | 47 | 0.0000 | 0.65 | 0.69 |
| S (VKDPS) | | | | | | | | | | |
| Vitronectin | ug/mL | 2395.32 | 466.22 | 2360.00 | 1550.00 | 3470.00 | 47 | 0.0000 | 1.34 | 1.33 |
| von Willebrand Factor (vWF) | ug/mL | 52.87 | 21.82 | 48.50 | 21.00 | 100.00 | 46 | 0.0010 | 1.32 | 1.31 |
| YKL-40 | ng/mL | 27.64 | 14.42 | 25.00 | 7.00 | 89.00 | 47 | 0.0105 | 0.79 | 0.89 |

TABLE 11
Phase II Clinical Trial - 31 analytes (biomarkers) which had >4 fold difference in the medians
(31 markers with 1) p < 0.05, 2) >4 folds change in mean or median between HIBM samples
and normal controls, therefore having a mean or median HIBM/normal >4 or <0.25, 47 HUBM Samples)

| | | | | | | | Positive | Normal |
|---|---|---|---|---|---|---|---|---|
| | Units | HIBM Mean | HIBM SD | HIBM Median | Min | Max | Count | Mean |
| B Lymphocyte | pg/mL | 70.50 | 4.50 | 70.50 | 66.00 | 75.00 | 2 | 19 |
| Chemoattractant | | | | | | | | |
| (BLC) | | | | | | | | |
| Brain-Derived | ng/mL | 19.72 | 4.80 | 20.00 | 8.80 | 31.00 | 47 | 0.57 |
| Neurotrophic Factor | | | | | | | | |
| (BDNF) | | | | | | | | |
| CD40 Ligand | ng/mL | 1.75 | 0.90 | 1.60 | 0.12 | 3.90 | 47 | 0.031 |
| (CD40-L) | | | | | | | | |
| Chromogranin-A | ng/mL | 344.73 | 164.73 | 327.00 | 30.00 | 871.00 | 41 | 79 |
| (CgA) | | | | | | | | |
| Creatine Kinase- | ng/mL | 12.36 | 10.04 | 8.70 | 3.30 | 47.00 | 47 | 1.1 |
| MB (CK-MB) | | | | | | | | |
| Eotaxin-1 | pg/mL | 207.05 | 39.57 | 209.00 | 148.00 | 299.00 | 21 | 42 |
| Epidermal Growth | pg/mL | 278.28 | 157.61 | 248.00 | 44.00 | 656.00 | 46 | 12 |
| Factor (EGF) | | | | | | | | |
| Epithelial-Derived | ng/mL | 2.46 | 1.34 | 2.00 | 0.67 | 6.40 | 47 | 0.38 |
| Neutrophil- | | | | | | | | |
| Activating Protein | | | | | | | | |
| 78 (ENA-78) | | | | | | | | |
| Glucagon-like | pg/mL | 16.99 | 13.62 | 11.50 | 7.60 | 60.00 | 16 | 2.8 |
| Peptide 1, total | | | | | | | | |
| (GLP-1 total) | | | | | | | | |
| Hepatocyte Growth | ng/mL | 14.18 | 8.41 | 13.00 | 2.80 | 35.00 | 47 | 3.1 |
| Factor (HGF) | | | | | | | | |
| Interleukin-12 | ng/mL | 0.50 | 0.14 | 0.48 | 0.30 | 0.91 | 36 | 0.15 |
| Subunit p40 (IL- | | | | | | | | |
| 12p40) | | | | | | | | |
| Kidney Injury | ng/mL | 0.02 | 0.01 | 0.02 | 0.02 | 0.05 | 14 | 0.16 |
| Molecule-1 (KIM- | | | | | | | | |
| 1) | | | | | | | | |
| Lactoylglutathione | ng/mL | 17.26 | 16.54 | 13.00 | 2.80 | 102.00 | 47 | 3.4 |
| lyase (LGL) | | | | | | | | |
| Latency-Associated | ng/mL | 10.63 | 3.05 | 11.00 | 5.00 | 19.00 | 47 | 1.1 |
| Peptide of | | | | | | | | |
| Transforming | | | | | | | | |
| Growth Factor beta | | | | | | | | |
| 1 (LAP TGF-b1) | | | | | | | | |
| Macrophage | ng/mL | 0.50 | 0.16 | 0.45 | 0.26 | 0.96 | 45 | 0.100 |
| Colony-Stimulating | | | | | | | | |
| Factor 1 (M-CSF) | | | | | | | | |
| Matrix | ng/mL | 7.93 | 4.92 | 6.90 | 1.10 | 21.00 | 47 | 1.2 |
| Metalloproteinase-1 | | | | | | | | |
| (MMP-1) | | | | | | | | |
| Matrix | ng/mL | 657.09 | 197.30 | 591.00 | 326.00 | 1310.00 | 47 | 119 |
| Metalloproteinase- | | | | | | | | |
| 9, total (MMP-9, | | | | | | | | |
| total) | | | | | | | | |
| Monocyte | pg/mL | 3361.28 | 1297.41 | 3370.00 | 1140.00 | 7310.00 | 47 | 707 |
| Chemotactic Protein | | | | | | | | |
| 4 (MCP-4) | | | | | | | | |
| Myeloperoxidase | ng/mL | 1045.60 | 901.05 | 741.00 | 193.00 | 4380.00 | 47 | 199 |
| (MPO) | | | | | | | | |
| Myoglobin | ng/mL | 152.09 | 112.88 | 100.00 | 47.00 | 551.00 | 47 | 15 |
| Neuron-Specific | ng/mL | 2.51 | 6.41 | 1.10 | 0.32 | 45.00 | 47 | 14 |
| Enolase (NSE) | | | | | | | | |
| Plasminogen | ng/mL | 196.30 | 56.14 | 191.00 | 88.00 | 316.00 | 47 | 32 |
| Activator Inhibitor | | | | | | | | |
| 1 (PAI-1) | | | | | | | | |
| Platelet-Derived | pg/mL | 15631.91 | 4560.00 | 15600.00 | 7260.00 | 24600.00 | 47 | 271 |
| Growth Factor BB | | | | | | | | |
| (PDGF-BB) | | | | | | | | |
| Proinsulin, Total | pM | 54.75 | 10.33 | 55.00 | 43.00 | 66.00 | 4 | 15 |
| Protein S100-A4 | ng/mL | 50.87 | 46.18 | 38.50 | 9.70 | 149.00 | 10 | 6.6 |
| (S100-A4) | | | | | | | | |
| Receptor tyrosine- | ng/mL | 0.62 | 0.69 | 0.31 | 0.18 | 3.30 | 47 | 2.2 |
| protein kinase erbB- | | | | | | | | |
| 3 (ErbB3) | | | | | | | | |
| Squamous Cell | ng/mL | 1.53 | 0.19 | 1.60 | 1.20 | 1.80 | 7 | 0.50 |
| Carcinoma Antigen- | | | | | | | | |
| 1 (SCCA-1) | | | | | | | | |
| T-Cell-Specific | ng/mL | 18.26 | 7.29 | 18.00 | 3.70 | 36.00 | 47 | 1.4 |
| Protein RANTES | | | | | | | | |
| (RANTES) | | | | | | | | |
| Thrombospondin-1 | ng/mL | 9800.64 | 2558.16 | 9540.00 | 3930.00 | 14700.00 | 47 | 876 |
| Tumor Necrosis | pg/mL | 33.00 | 7.12 | 37.00 | 23.00 | 39.00 | 3 | 6.0 |
| Factor alpha (TNF- | | | | | | | | |
| alpha) | | | | | | | | |
| Vascular | ng/mL | 35.32 | 6.73 | 36.00 | 22.00 | 46.00 | 47 | 3.9 |
| Endothelial Growth | | | | | | | | |
| Factor C (VEGF-C) | | | | | | | | |

| | | | | | | | change | change |
|---|---|---|---|---|---|---|---|---|
| | | | | | Normal | | in | in |
| | | | Normal | Normal | Controls | | mean | median |
| | Normal | Normal | Controls | Controls | Positive | p- | (HIBM/ | (HIBM/ |
| | SD | Median | Min | Max | Count | value | nomral) | normal) |
| B Lymphocyte | 42 | 8.6 | 6 | 245 | 67 | 0.0006 | 3.63 | 8.17 |
| Chemoattractant | | | | | | | | |
| (BLC) | | | | | | | | |
| Brain-Derived | 0.69 | 0.40 | 0 | 7 | 126 | 0.0000 | 34.58 | 50.06 |
| Neurotrophic Factor | | | | | | | | |
| (BDNF) | | | | | | | | |
| CD40 Ligand | 0.064 | 0.019 | 0 | 0 | 54 | 0.0000 | 57.13 | 82.90 |
| (CD40-L) | | | | | | | | |
| Chromogranin-A | 113 | 13 | 5 | 372 | 52 | 0.0000 | 4.36 | 25.95 |
| (CgA) | | | | | | | | |
| Creatine Kinase- | 0.93 | 0.80 | 0 | 6 | 125 | 0.0000 | 11.37 | 10.90 |
| MB (CK-MB) | | | | | | | | |
| Eotaxin-1 | 30 | 28 | 11 | 202 | 86 | 0.0000 | 4.90 | 7.39 |
| Epidermal Growth | 44 | 4.7 | 2 | 432 | 102 | 0.0000 | 23.55 | 53.22 |
| Factor (EGF) | | | | | | | | |
| Epithelial-Derived | 0.39 | 0.29 | 0 | 4 | 126 | 0.0000 | 6.42 | 6.85 |
| Neutrophil- | | | | | | | | |
| Activating Protein | | | | | | | | |
| 78 (ENA-78) | | | | | | | | |
| Glucagon-like | 2.0 | 2.4 | 1 | 9 | 52 | 0.0011 | 6.09 | 4.71 |
| Peptide 1, total | | | | | | | | |
| (GLP-1 total) | | | | | | | | |
| Hepatocyte Growth | 2.1 | 2.6 | 0 | 11 | 123 | 0.0000 | 4.59 | 5.04 |
| Factor (HGF) | | | | | | | | |
| Interleukin-12 | 0.051 | 0.12 | 0 | 0 | 25 | 0.0000 | 3.41 | 4.14 |
| Subunit p40 (IL- | | | | | | | | |
| 12p40) | | | | | | | | |
| Kidney Injury | 0.34 | 0.089 | 0 | 3 | 126 | 0.0000 | 0.16 | 0.25 |
| Molecule-1 (KIM- | | | | | | | | |
| 1) | | | | | | | | |
| Lactoylglutathione | 1.4 | 3.1 | 0 | 11 | 126 | 0.0000 | 5.04 | 4.21 |
| lyase (LGL) | | | | | | | | |
| Latency-Associated | 0.55 | 0.99 | 0 | 5 | 126 | 0.0000 | 9.75 | 11.14 |
| Peptide of | | | | | | | | |
| Transforming | | | | | | | | |
| Growth Factor beta | | | | | | | | |
| 1 (LAP TGF-b1) | | | | | | | | |
| Macrophage | 0.033 | 0.092 | 0 | 0 | 126 | 0.0000 | 5.02 | 4.90 |
| Colony-Stimulating | | | | | | | | |
| Factor 1 (M-CSF) | | | | | | | | |
| Matrix | 0.97 | 0.95 | 0 | 10 | 117 | 0.0000 | 6.89 | 7.24 |
| Metalloproteinase-1 | | | | | | | | |
| (MMP-1) | | | | | | | | |
| Matrix | 54 | 114 | 42 | 569 | 126 | 0.0000 | 5.51 | 5.18 |
| Metalloproteinase- | | | | | | | | |
| 9, total (MMP-9, | | | | | | | | |
| total) | | | | | | | | |
| Monocyte | 272 | 697 | 146 | 1660 | 126 | 0.0000 | 4.75 | 4.84 |
| Chemotactic Protein | | | | | | | | |
| 4 (MCP-4) | | | | | | | | |
| Myeloperoxidase | 139 | 158 | 46 | 716 | 124 | 0.0000 | 5.24 | 4.69 |
| (MPO) | | | | | | | | |
| Myoglobin | 12 | 11 | 4 | 89 | 126 | 0.0000 | 10.16 | 8.81 |
| Neuron-Specific | 11 | 10 | 2 | 84 | 112 | 0.0000 | 0.18 | 0.11 |
| Enolase (NSE) | | | | | | | | |
| Plasminogen | 29 | 23 | 4 | 166 | 126 | 0.0000 | 6.12 | 8.16 |
| Activator Inhibitor | | | | | | | | |
| 1 (PAI-1) | | | | | | | | |
| Platelet-Derived | 327 | 167 | 42 | 2390 | 79 | 0.0000 | 57.62 | 93.41 |
| Growth Factor BB | | | | | | | | |
| (PDGF-BB) | | | | | | | | |
| Proinsulin, Total | 15 | 11 | 4 | 93 | 68 | 0.0046 | 3.61 | 5.14 |
| Protein S100-A4 | 7.8 | 4.6 | 0 | 63 | 102 | 0.0183 | 7.73 | 8.44 |
| (S100-A4) | | | | | | | | |
| Receptor tyrosine- | 0.85 | 2.3 | 0 | 4 | 126 | 0.0000 | 0.29 | 0.14 |
| protein kinase erbB- | | | | | | | | |
| 3 (ErbB3) | | | | | | | | |
| Squamous Cell | 0.65 | 0.36 | 0 | 6 | 125 | 0.0000 | 3.05 | 4.47 |
| Carcinoma Antigen- | | | | | | | | |
| 1 (SCCA-1) | | | | | | | | |
| T-Cell-Specific | 1.7 | 1.1 | 0 | 14 | 126 | 0.0000 | 12.69 | 17.14 |
| Protein RANTES | | | | | | | | |
| (RANTES) | | | | | | | | |
| Thrombospondin-1 | 1160 | 584 | 44 | 8460 | 125 | 0.0000 | 11.19 | 16.34 |
| Tumor Necrosis | 6.3 | 3.8 | 2 | 37 | 62 | 0.0304 | 5.47 | 9.66 |
| Factor alpha (TNF- | | | | | | | | |
| alpha) | | | | | | | | |
| Vascular | 1.0 | 3.8 | 2 | 9 | 126 | 0.0000 | 9.07 | 9.60 |
| Endothelial Growth | | | | | | | | |
| Factor C (VEGF-C) | | | | | | | | |

TABLE 12
Phase II Clinical Trial - 16 analytes (biomarkers) which had >50% of HIBM and normal
samples are above LDD or greater than 5 fold changes in median between HIBM samples and
normal controls, 3) >50% of HIBM and normal samples above least detectable dose (LDD).

| | | | | | | | Positive | Normal |
|---|---|---|---|---|---|---|---|---|
| | Units | HIBM Mean | HIBM SD | HIBM Median | Min | Max | Count | Mean |
| Brain-Derived | ng/mL | 19.72 | 4.80 | 20.00 | 8.80 | 31.00 | 47 | 0.57 |
| Neurotrophic Factor | | | | | | | | |
| (BDNF) | | | | | | | | |
| Creatine Kinase- | ng/mL | 12.36 | 10.04 | 8.70 | 3.30 | 47.00 | 47 | 1.1 |
| MB (CK-MB) | | | | | | | | |
| Epidermal Growth | pg/mL | 278.28 | 157.61 | 248.00 | 44.00 | 656.00 | 46 | 12 |
| Factor (EGF) | | | | | | | | |
| Epithelial-Derived | ng/mL | 2.46 | 1.34 | 2.00 | 0.67 | 6.40 | 47 | 0.38 |
| Neutrophil- | | | | | | | | |
| Activating Protein | | | | | | | | |
| 78 (ENA-78) | | | | | | | | |
| Hepatocyte Growth | ng/mL | 14.18 | 8.41 | 13.00 | 2.80 | 35.00 | 47 | 3.1 |
| Factor (HGF) | | | | | | | | |
| Latency-Associated | ng/mL | 10.63 | 3.05 | 11.00 | 5.00 | 19.00 | 47 | 1.1 |
| Peptide of | | | | | | | | |
| Transforming | | | | | | | | |
| Growth Factor beta | | | | | | | | |
| 1 (LAP TGF-b1) | | | | | | | | |
| Matrix | ng/mL | 7.93 | 4.92 | 6.90 | 1.10 | 21.00 | 47 | 1.2 |
| Metalloproteinase-1 | | | | | | | | |
| (MMP-1) | | | | | | | | |
| Matrix | ng/mL | 657.09 | 197.30 | 591.00 | 326.00 | 1310.00 | 47 | 119 |
| Metalloproteinase- | | | | | | | | |
| 9, total (MMP-9, | | | | | | | | |
| total) | | | | | | | | |
| Myoglobin | ng/mL | 152.09 | 112.88 | 100.00 | 47.00 | 551.00 | 47 | 15 |
| Neuron-Specific | ng/mL | 2.51 | 6.41 | 1.10 | 0.32 | 45.00 | 47 | 14 |
| Enolase (NSE) | | | | | | | | |
| Plasminogen | ng/mL | 196.30 | 56.14 | 191.00 | 88.00 | 316.00 | 47 | 32 |
| Activator Inhibitor | | | | | | | | |
| 1 (PAI-1) | | | | | | | | |
| Platelet-Derived | pg/mL | 15631.91 | 4560.00 | 15600.00 | 7260.00 | 24600.00 | 47 | 271 |
| Growth Factor BB | | | | | | | | |
| (PDGF-BB) | | | | | | | | |
| Receptor tyrosine- | ng/mL | 0.62 | 0.69 | 0.31 | 0.18 | 3.30 | 47 | 2.2 |
| protein kinase erbB- | | | | | | | | |
| 3 (ErbB3) | | | | | | | | |
| T-Cell-Specific | ng/mL | 18.26 | 7.29 | 18.00 | 3.70 | 36.00 | 47 | 1.4 |
| Protein RANTES | | | | | | | | |
| (RANTES) | | | | | | | | |
| Thrombospondin-1 | ng/mL | 9800.64 | 2558.16 | 9540.00 | 3930.00 | 14700.00 | 47 | 876 |
| Vascular | ng/mL | 35.32 | 6.73 | 36.00 | 22.00 | 46.00 | 47 | 3.9 |
| Endothelial Growth | | | | | | | | |
| Factor C (VEGF-C) | | | | | | | | |

| | | | | | | | change | change |
|---|---|---|---|---|---|---|---|---|
| | | | | | Normal | | in | in |
| | | | Normal | Normal | Controls | | mean | median |
| | Normal | Normal | Controls | Controls | Positive | p- | (HIBM/ | (HIBM/ |
| | SD | Median | Min | Max | Count | value | nomral) | normal) |
| Brain-Derived | 0.69 | 0.40 | 0 | 7 | 126 | 0.0000 | 34.58 | 50.06 |
| Neurotrophic Factor | | | | | | | | |
| (BDNF) | | | | | | | | |
| Creatine Kinase- | 0.93 | 0.80 | 0 | 6 | 125 | 0.0000 | 11.37 | 10.90 |
| MB (CK-MB) | | | | | | | | |
| Epidermal Growth | 44 | 4.7 | 2 | 432 | 102 | 0.0000 | 23.55 | 53.22 |
| Factor (EGF) | | | | | | | | |
| Epithelial-Derived | 0.39 | 0.29 | 0 | 4 | 126 | 0.0000 | 6.42 | 6.85 |
| Neutrophil- | | | | | | | | |
| Activating Protein | | | | | | | | |
| 78 (ENA-78) | | | | | | | | |
| Hepatocyte Growth | 2.1 | 2.6 | 0 | 11 | 123 | 0.0000 | 4.59 | 5.04 |
| Factor (HGF) | | | | | | | | |
| Latency-Associated | 0.55 | 0.99 | 0 | 5 | 126 | 0.0000 | 9.75 | 11.14 |
| Peptide of | | | | | | | | |
| Transforming | | | | | | | | |
| Growth Factor beta | | | | | | | | |
| 1 (LAP TGF-b1) | | | | | | | | |
| Matrix | 0.97 | 0.95 | 0 | 10 | 117 | 0.0000 | 6.89 | 7.24 |
| Metalloproteinase-1 | | | | | | | | |
| (MMP-1) | | | | | | | | |
| Matrix | 54 | 114 | 42 | 569 | 126 | 0.0000 | 5.51 | 5.18 |
| Metalloproteinase- | | | | | | | | |
| 9, total (MMP-9, | | | | | | | | |
| total) | | | | | | | | |
| Myoglobin | 12 | 11 | 4 | 89 | 126 | 0.0000 | 10.16 | 8.81 |
| Neuron-Specific | 11 | 10 | 2 | 84 | 112 | 0.0000 | 0.18 | 0.11 |
| Enolase (NSE) | | | | | | | | |
| Plasminogen | 29 | 23 | 4 | 166 | 126 | 0.0000 | 6.12 | 8.16 |
| Activator Inhibitor | | | | | | | | |
| 1 (PAI-1) | | | | | | | | |
| Platelet-Derived | 327 | 167 | 42 | 2390 | 79 | 0.0000 | 57.62 | 93.41 |
| Growth Factor BB | | | | | | | | |
| (PDGF-BB) | | | | | | | | |
| Receptor tyrosine- | 0.85 | 2.3 | 0 | 4 | 126 | 0.0000 | 0.29 | 0.14 |
| protein kinase erbB- | | | | | | | | |
| 3 (ErbB3) | | | | | | | | |
| T-Cell-Specific | 1.7 | 1.1 | 0 | 14 | 126 | 0.0000 | 12.69 | 17.14 |
| Protein RANTES | | | | | | | | |
| (RANTES) | | | | | | | | |
| Thrombospondin-1 | 1160 | 584 | 44 | 8460 | 125 | 0.0000 | 11.19 | 16.34 |
| Vascular | 1.0 | 3.8 | 2 | 9 | 126 | 0.0000 | 9.07 | 9.60 |
| Endothelial Growth | | | | | | | | |
| Factor C (VEGF-C) | | | | | | | | |

TABLE 13
Phase II Clinical Trial - 16 markers with 1) p < 0.05, 2) >5 folds change
in median between HIBM samples and normal controls, 3) >50% of HIBM and normal
samples above least detectable dose (LDD), 4) >80% of HIBM samples above 5x LDD.

| | | HIBM | HIBM | HIBM | | | Positive | Normal | Normal |
|---|---|---|---|---|---|---|---|---|---|
| | Units | Mean | SD | Median | Min | Max | Count | Mean | SD |
| Brain-Derived | ng/mL | 19.72 | 4.80 | 20.00 | 8.80 | 31.00 | 47 | 0.57 | 0.69 |
| Neurotrophic | | | | | | | | | |
| Factor | | | | | | | | | |
| (BDNF) | | | | | | | | | |
| Creatine | ng/mL | 12.36 | 10.04 | 8.70 | 3.30 | 47.00 | 47 | 1.1 | 0.93 |
| Kinase-MB | | | | | | | | | |
| (CK-MB) | | | | | | | | | |
| Epidermal | pg/mL | 278.28 | 157.61 | 248.00 | 44.00 | 656.00 | 46 | 12 | 44 |
| Growth Factor | | | | | | | | | |
| (EGF) | | | | | | | | | |
| Epithelial- | ng/mL | 2.46 | 1.34 | 2.00 | 0.67 | 6.40 | 47 | 0.38 | 0.39 |
| Derived | | | | | | | | | |
| Neutrophil- | | | | | | | | | |
| Activating | | | | | | | | | |
| Protein 78 | | | | | | | | | |
| (ENA-78) | | | | | | | | | |
| Hepatocyte | ng/mL | 14.18 | 8.41 | 13.00 | 2.80 | 35.00 | 47 | 3.1 | 2.1 |
| Growth Factor | | | | | | | | | |
| (HGF) | | | | | | | | | |
| Latency- | ng/mL | 10.63 | 3.05 | 11.00 | 5.00 | 19.00 | 47 | 1.1 | 0.55 |
| Associated | | | | | | | | | |
| Peptide of | | | | | | | | | |
| Transforming | | | | | | | | | |
| Growth Factor | | | | | | | | | |
| beta 1 (LAP | | | | | | | | | |
| TGF-b1) | | | | | | | | | |
| Matrix | ng/mL | 7.93 | 4.92 | 6.90 | 1.10 | 21.00 | 47 | 1.2 | 0.97 |
| Metalloproteinase-1 | | | | | | | | | |
| (MMP-1) | | | | | | | | | |
| Matrix | ng/mL | 657.09 | 197.30 | 591.00 | 326.00 | 1310.00 | 47 | 119 | 54 |
| Metalloproteinase-9, | | | | | | | | | |
| total (MMP-9, | | | | | | | | | |
| total) | | | | | | | | | |
| Myoglobin | ng/mL | 152.09 | 112.88 | 100.00 | 47.00 | 551.00 | 47 | 15 | 12 |
| Plasminogen | ng/mL | 196.30 | 56.14 | 191.00 | 88.00 | 316.00 | 47 | 32 | 29 |
| Activator | | | | | | | | | |
| Inhibitor 1 | | | | | | | | | |
| (PAI-1) | | | | | | | | | |
| Platelet- | pg/mL | 15631.91 | 4560.00 | 15600.00 | 7260.00 | 24600.00 | 47 | 271 | 327 |
| Derived | | | | | | | | | |
| Growth Factor | | | | | | | | | |
| BB (PDGF- | | | | | | | | | |
| BB) | | | | | | | | | |
| Receptor | ng/mL | 0.62 | 0.69 | 0.31 | 0.18 | 3.30 | 47 | 2.2 | 0.85 |
| tyrosine- | | | | | | | | | |
| protein kinase | | | | | | | | | |
| erbB-3 | | | | | | | | | |
| (ErbB3) | | | | | | | | | |
| T-Cell- | ng/mL | 18.26 | 7.29 | 18.00 | 3.70 | 36.00 | 47 | 1.4 | 1.7 |
| Specific | | | | | | | | | |
| Protein | | | | | | | | | |
| RANTES | | | | | | | | | |
| (RANTES) | | | | | | | | | |
| Thrombospondin-1 | ng/mL | 9800.64 | 2558.16 | 9540.00 | 3930.00 | 14700.00 | 47 | 876 | 1160 |
| Vascular | ng/mL | 35.32 | 6.73 | 36.00 | 22.00 | 46.00 | 47 | 3.9 | 1.0 |
| Endothelial | | | | | | | | | |
| Growth Factor | | | | | | | | | |
| C (VEGF-C) | | | | | | | | | |

| | | | | | | change | change | | |
|---|---|---|---|---|---|---|---|---|---|
| | | | | | | in | in | | |
| | | | | | | mean | median | Myriad | |
| | Normal | | | Positive | p- | (HIBM/ | (HIBM/ | RBM | |
| | Median | Min | Max | Count | value | nomral) | normal) | LDD | 5xLDD |
| Brain-Derived | 0.40 | 0 | 7 | 126 | 0.0000 | 34.58 | 50.06 | 0.023 | 0.12 |
| Neurotrophic | | | | | | | | | |
| Factor | | | | | | | | | |
| (BDNF) | | | | | | | | | |
| Creatine | 0.80 | 0 | 6 | 125 | 0.0000 | 11.37 | 10.90 | 0.91 | 4.55 |
| Kinase-MB | | | | | | | | | |
| (CK-MB) | | | | | | | | | |
| Epidermal | 4.7 | 2 | 432 | 102 | 0.0000 | 23.55 | 53.22 | 3.0 | 15.00 |
| Growth Factor | | | | | | | | | |
| (EGF) | | | | | | | | | |
| Epithelial- | 0.29 | 0 | 4 | 126 | 0.0000 | 6.42 | 6.85 | 0.033 | 0.17 |
| Derived | | | | | | | | | |
| Neutrophil- | | | | | | | | | |
| Activating | | | | | | | | | |
| Protein 78 | | | | | | | | | |
| (ENA-78) | | | | | | | | | |
| Hepatocyte | 2.6 | 0 | 11 | 123 | 0.0000 | 4.59 | 5.04 | 0.20 | 1.00 |
| Growth Factor | | | | | | | | | |
| (HGF) | | | | | | | | | |
| Latency- | 0.99 | 0 | 5 | 126 | 0.0000 | 9.75 | 11.14 | 0.056 | 0.28 |
| Associated | | | | | | | | | |
| Peptide of | | | | | | | | | |
| Transforming | | | | | | | | | |
| Growth Factor | | | | | | | | | |
| beta 1 (LAP | | | | | | | | | |
| TGF-b1) | | | | | | | | | |
| Matrix | 0.95 | 0 | 10 | 117 | 0.0000 | 6.89 | 7.24 | 0.45 | 2.25 |
| Metalloproteinase-1 | | | | | | | | | |
| (MMP-1) | | | | | | | | | |
| Matrix | 114 | 42 | 569 | 126 | 0.0000 | 5.51 | 5.18 | 1.2 | 6.00 |
| Metalloproteinase-9, | | | | | | | | | |
| total (MMP-9, | | | | | | | | | |
| total) | | | | | | | | | |
| Myoglobin | 11 | 4 | 89 | 126 | 0.0000 | 10.16 | 8.81 | 2.0 | 10.00 |
| Plasminogen | 23 | 4 | 166 | 126 | 0.0000 | 6.12 | 8.16 | 1.1 | 5.50 |
| Activator | | | | | | | | | |
| Inhibitor 1 | | | | | | | | | |
| (PAI-1) | | | | | | | | | |
| Platelet- | 167 | 42 | 2390 | 79 | 0.0000 | 57.62 | 93.41 | 127 | 635.00 |
| Derived | | | | | | | | | |
| Growth Factor | | | | | | | | | |
| BB (PDGF- | | | | | | | | | |
| BB) | | | | | | | | | |
| Receptor | 2.3 | 0 | 4 | 126 | 0.0000 | 0.29 | 0.14 | 0.030 | 0.15 |
| tyrosine- | | | | | | | | | |
| protein kinase | | | | | | | | | |
| erbB-3 | | | | | | | | | |
| (ErbB3) | | | | | | | | | |
| T-Cell- | 1.1 | 0 | 14 | 126 | 0.0000 | 12.69 | 17.14 | 0.033 | 0.17 |
| Specific | | | | | | | | | |
| Protein | | | | | | | | | |
| RANTES | | | | | | | | | |
| (RANTES) | | | | | | | | | |
| Thrombospondin-1 | 584 | 44 | 8460 | 125 | 0.0000 | 11.19 | 16.34 | 57 | 285.00 |
| Vascular | 3.8 | 2 | 9 | 126 | 0.0000 | 9.07 | 9.60 | 1.3 | 6.50 |
| Endothelial | | | | | | | | | |
| Growth Factor | | | | | | | | | |
| C (VEGF-C) | | | | | | | | | |

Analytes were excluded from analysis if <50% of the HIBM patient samples were below least detectable dose (LDD). Within each dosage group, individual values with >5× difference to the mean of the dosage group were considered as outliers and thus removed from analysis.

Out of the 32 analytes, the levels of eleven (11) analytes showed signs of improvement at week 24 (see Table 14), mainly moving toward normal levels (normalization) as defined by the 126 normal controls used in the study. The 6 g/day group also showed a more favorable change in the levels of these analytes compared to the placebo group at week 24.

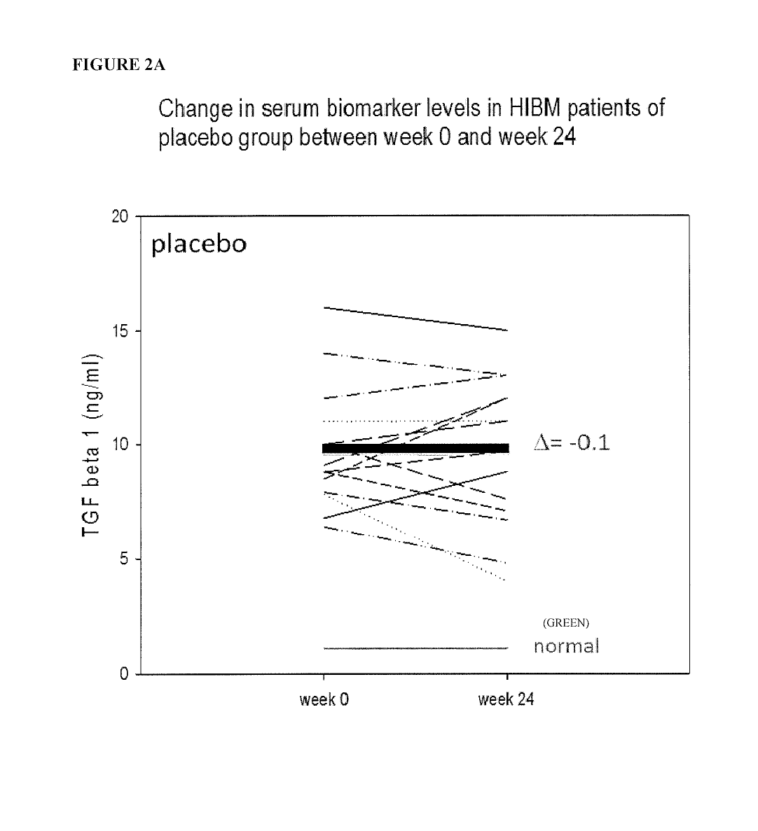

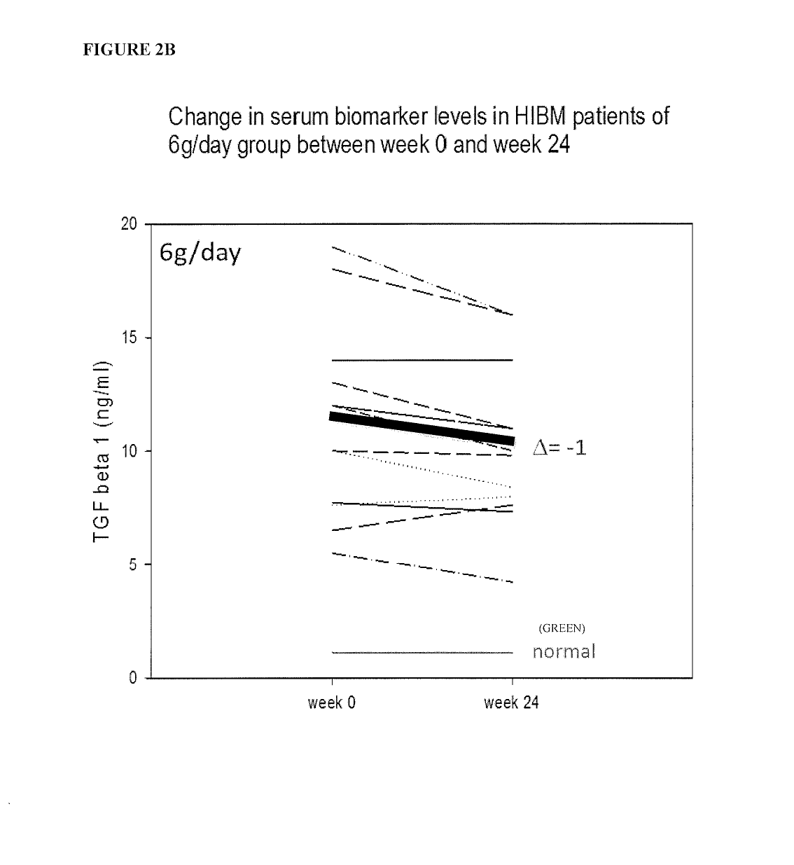

The analytes (biomarkers) with improvement in levels are summarized in Table 13. Upon treatment, levels of these analytes show an overall trend of either stabilization or normalization. As used herein, the term normalization indicates that the level of a given biomarker after drug treatment moves toward a normal level, and the change in is larger compared to that of a placebo treatment. As used herein, the term stabilization indicates that the level of a given biomarker after drug treatment may still move away from normal level, but at a slower rate compared to that of a placebo treatment. For example, LAP TGF-b1 is an example of biomarker showing trend of normalization when the baseline level in HIBM patients is higher than normal controls, see FIG. 2. NCAM is another example of biomarker showing trend of normalization when the baseline level in HIBM patients is lower than normal controls, see FIG. 3.

TABLE 14
Summary of biomarkers showing improvement in levels at week 24

| | | fold change | | | | |
|---|---|---|---|---|---|---|
| | | between | average | average | average | |
| | normal | baseline and | Δweek 0- | Δweek 0- | Δweek 0- | |
| Analytes | level | normals | 24 (placebo) | 24 (3 g/day) | 24 (6 g/day) | overall trend |
| Chromogranin-A | 79 | 4.4 | 31.67 | 64.00 | 5.83 | stabilization |
| (CgA) (ng/mL) | | | | | | |
| Epithelial-Derived | 0.38 | 6.4 | −0.41 | −0.26 | −0.42 | normalization |
| Neutrophil-Activating | | | | | | |
| Protein 78 (ENA-78) | | | | | | |
| (ng/mL) | | | | | | |
| Lactoylglutathione | 3.4 | 5 | −3.53 | 0.25 | −8.27 | normalization |
| lyase (LGL) (ng/mL) | | | | | | |
| Latency-Associated | 1.1 | 9.8 | −0.10 | −0.44 | −1.00 | normalization |
| Peptide of | | | | | | |
| Transforming Growth | | | | | | |
| Factor beta 1 (LAP | | | | | | |
| TGF-b1) (ng/mL) | | | | | | |
| Macrophage Colony- | 0.100 | 5 | 0.08 | 0.10 | 0.02 | stabilization |
| Stimulating Factor 1 | | | | | | |
| (M-CSF) (ng/mL) | | | | | | |
| Matrix | 119 | 5.5 | 534.93 | 449.18 | 444.64 | stabilization |
| Metalloproteinase-9, | | | | | | |
| total (MMP-9, total) | | | | | | |
| (ng/mL) | | | | | | |
| Myoglobin (ng/mL) | 15 | 10.2 | −21.64 | −48.88 | −38.00 | normalization |
| Neuronal Cell | 1.6 | −1.9 | −0.17 | −0.07 | 0.02 | normalization |
| Adhesion Molecule | | | | | | |
| (Nr-CAM) (ng/mL) | | | | | | |
| Plasminogen | 32 | 6.1 | 4.14 | 0.82 | −0.36 | normalization |
| Activator Inhibitor 1 | | | | | | |
| (PAI-1) (ng/mL) | | | | | | |
| Receptor tyrosine- | 2.2 | −3.5 | −0.39 | −0.28 | −0.16 | stabilization |
| protein kinase erbB-3 | | | | | | |
| (ErbB3) (ng/mL) | | | | | | |
| Vascular Endothelial | 3.9 | 9.1 | 6.57 | 1.35 | 6.43 | stabilization |
| Growth Factor C | | | | | | |
| (VEGF-C) (ng/mL) | | | | | | |

normalization: level moved toward normal level and the change in 6 g/day group was larger compared to placebo
stabilization: level might still move away from normal level but at a slower rate in 6 g/day group compared to placebo

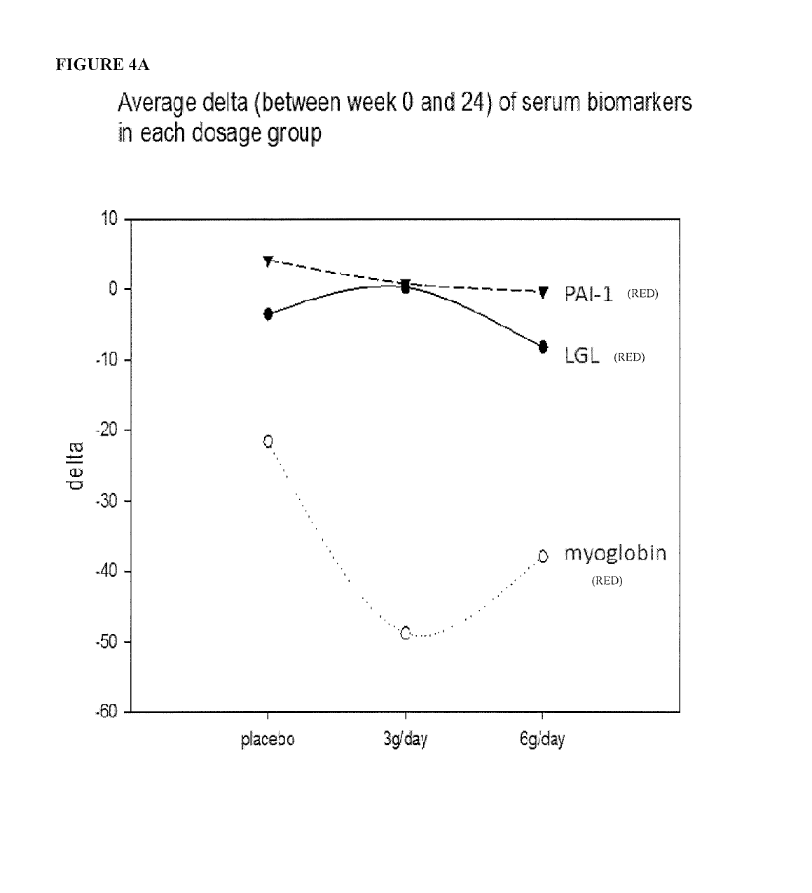

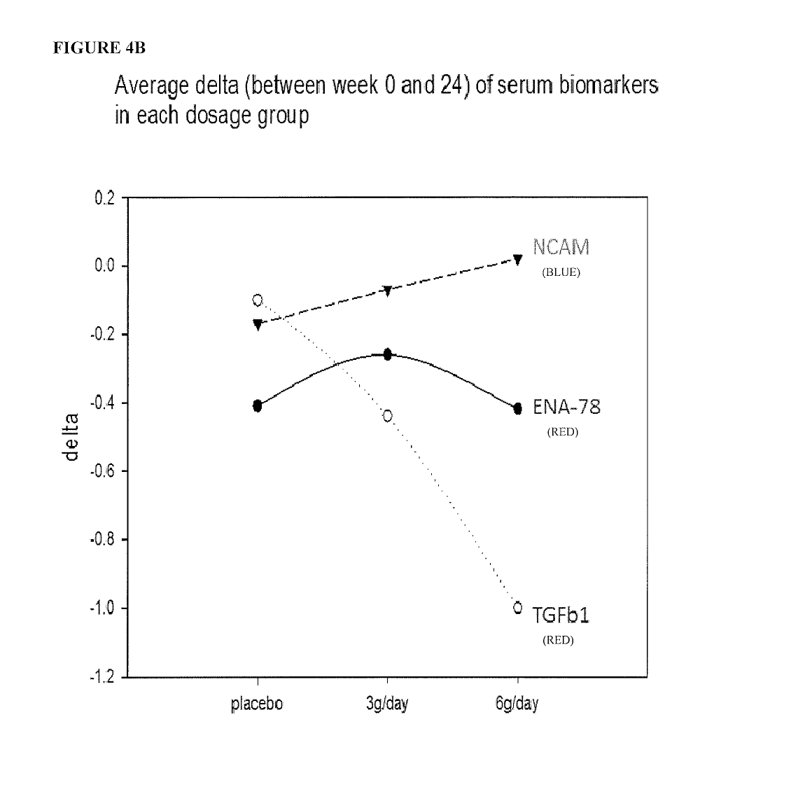

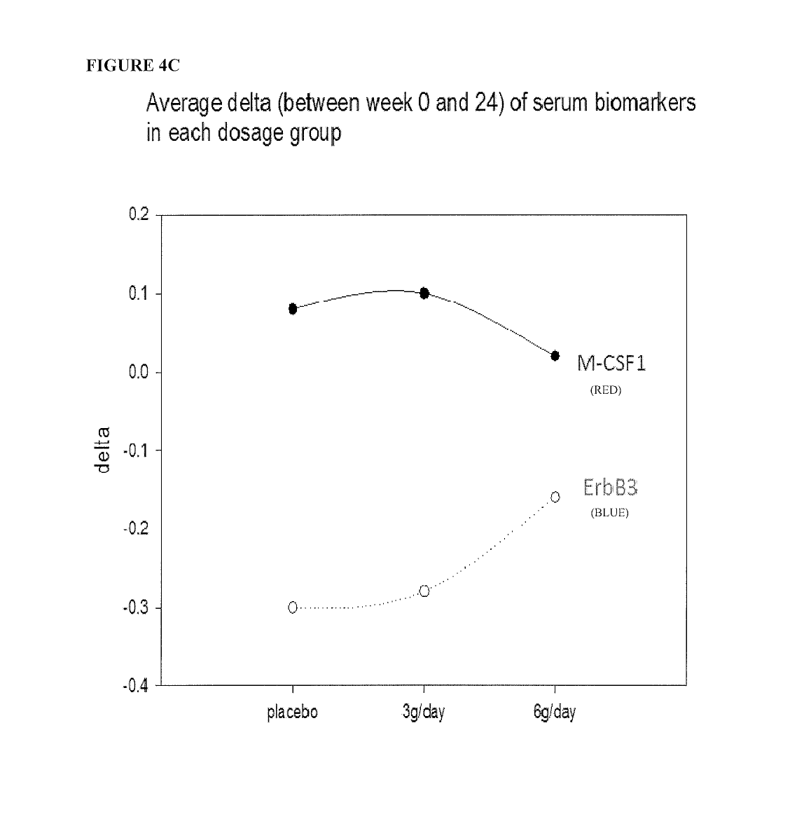

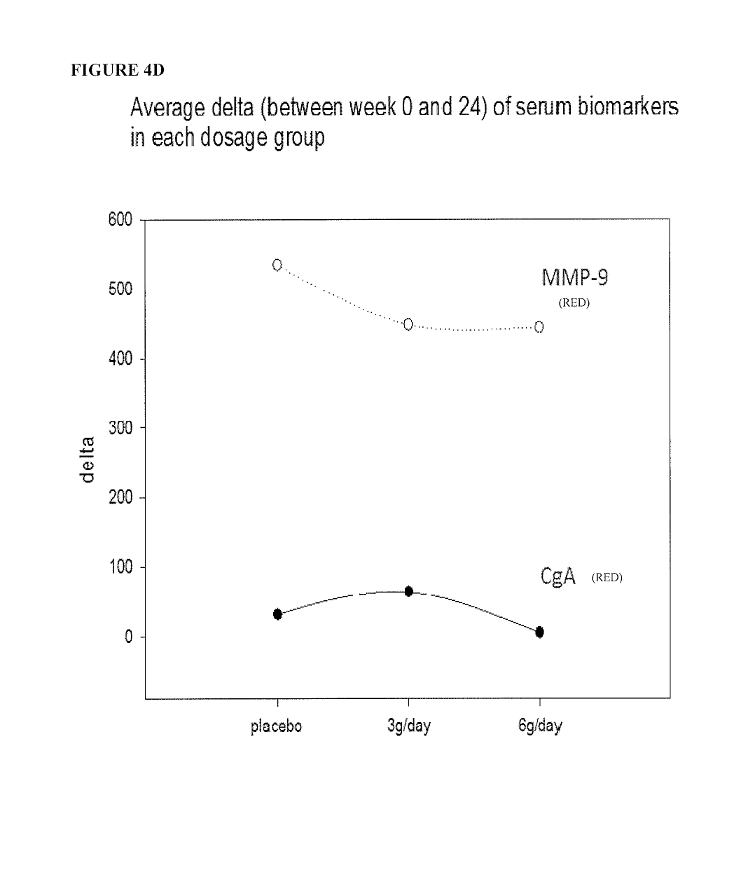

FIG. 4 shows Delta (A) of serum biomarkers between week 0 and week 24 in each dosage group (placebo, 3 g/day, and 6 g/day). Analytes with baseline levels in HIBM patients higher than normal controls include PAI-1, LGL, myoglobin, ENA-78, TGfb1, M-CSF1, MMP-9, and CgA. For these biomarkers, the slope of delta displays a downward trend from placebo to higher dosage group (x-axis), indicating a more favorable change in the higher dosage group moving toward normal levels. In contrast, analytes with baseline levels lower than normal controls include NCAM and ErbB3, the slope of delta always displayed a upward trend.

### Example 3

### Phase 2 Biomarkers

Forty-seven (47) HIBM patient serum samples from phase 2 study were tested using the Human Discovery MAP250+ quantitative immunoassay system developed by Myriad RBM.

The results of analysis are shown in FIGS. 1-6 (phase 1) of 61/779,929 filed on Mar. 13, 2013, and FIGS. 8-13 (phase 2) of 61/779,929 filed on Mar. 13, 2013, which is herein incorporated by reference in its entirety for all purposes. The same criteria used to narrow down the list of potential biomarkers of interest in the phase 1 study were applied to the phase 2 patients.

A summary of the comparison between the results for phase 1 and phase 2 patients using the same criteria to narrow down the potential list of biomarkers of interest is shown in Table 14 below.

TABLE 15
Phase 1 and Phase 2 Comparison

| | | | same markers |
|---|---|---|---|
| | | | identified |
| | phase 2 | phase 1 | in both |
| | patients | patients | phase 1 and 2 |
| | n = 47 | n = 18 | patients |
| # of markers tested | 243 | 258 | 238* |
| 1st cutoff: markers had a p-value | 140 | 134 | 89 |
| of <0.05 | | | |
| 2nd cutoff: markers with >4 folds | 31 | 34 | 20 |
| change in mean or median | | | |
| 3rd cutoff: markers in which >50% | 16 | 18 | 12 |
| of HIBM and normal samples above | | | |
| least detectable dose (LDD), or >5 | | | |
| folds change in median. | | | |
| 4th cutoff: markers with >5 folds | 15 | 11 | 9 |
| change in median. Also >80% of | | | |
| HIBM samples were above 5x LDD. | | | |

*20 markers discontinued and 5 additional markers offered for the phase 2 patients study

1. A method for monitoring responsiveness or efficacy of treatment with a sialic acid deficiency treatment in a subject suffering from a sialic acid deficiency comprising detecting the level of one or more sialic acid replacement therapy (SAT) biomarkers in a biological sample from the subject treated for a sialic acid deficiency, wherein an increase or decrease in the level of one or more SAT biomarkers compared to a predetermined standard level indicates efficacy of treatment with the sialic acid deficiency treatment.

2. A method for determining the treatment regimen for treating a sialic acid deficiency comprising detecting the level of one or more SAT biomarkers in a biological sample from a subject treated for a sialic acid deficiency and determining a treatment regimen based on an increase or decrease in the level of one or more SAT biomarkers in the biological sample compared to a predetermined standard level.

3. A method for predicting the treatment efficacy of a sialic acid deficiency treatment, comprising detecting the level of one or more SAT biomarkers in a biological sample from a subject, wherein an increase or decrease in the level of one or more SAT biomarkers compared to a predetermined standard level is predictive of the treatment efficacy of the sialic acid deficiency treatment.

4. A method for determining whether a subject with sialic acid deficiency is suitable for a sialic acid deficiency treatment comprising
detecting the level of one or more SAT biomarkers in a sample from the subject,
wherein an increase or decrease in the level of one or more SAT biomarkers compared to a predetermined standard level indicates a subject is suitable for the sialic acid deficiency treatment.

5. A method for treating a subject with a sialic acid deficiency comprising
detecting the level of one or more SAT biomarkers in a biological sample from the subject,
administering a sialic acid deficiency treatment to the subject if the level of one or more SAT biomarkers is increased or decreased compared to a predetermined standard level.

6. A method for treating a subject with a sialic acid deficiency comprising receiving information on the level of one or more SAT biomarkers in a biological sample from the subject, and administering a sialic acid deficiency treatment to the subject if the level of one or more SAT biomarkers is increased or decreased compared to a predetermined standard level.

7. A method for providing data comprising:
detecting the level of one or more SAT biomarkers in a sample from a subject, and
providing the information regarding the level of one or more SAT biomarkers to a healthcare provider for diagnosis or treatment of the subject.

8. The method of claim 7 further comprising receiving the biological sample from the healthcare provider before the detecting step.

9. A method of providing useful information for monitoring, predicting or determining the treatment efficacy of a sialic acid deficiency treatment comprising
determining the level of one or more SAT biomarkers from a biological sample of a subject, and
providing the level of one or more SAT biomarkers to an entity that provides a prediction or determination of the treatment efficacy based on an increase or decrease in the level of one or more of the SAT biomarkers compared to a predetermined standard level.

10. The method of any of claims 1-10, wherein the baseline level of one or more SAT biomarkers is detected before the sialic acid deficiency treatment, and an increase or decrease in the baseline level of one or more of the SAT biomarkers is compared to the predetermined standard level.

11. The method of any of claims 1-10, wherein the level of one or more SAT biomarkers is detected after at least one administration of the sialic acid deficiency treatment, and then compared to the predetermined standard level.

12. The method of claim 11, wherein the level of one or more SAT biomarkers is further compared to the level of the same SAT biomarkers in a biological sample of subjected treated with placebo.

13. The method of claim 11, wherein the level of one or more SAT biomarkers is detected after the at least one administration but before any obvious alteration of symptoms associated with the sialic acid deficiency.

14. The method of claim 1, wherein the presence or absence of a normalization or stabilization in the level of one or more SAT biomarkers toward a predetermined standard level after the treatment indicates efficacy of treatment with the sialic acid deficiency treatment.

15. The method of claim 2, wherein a treatment regimen is determined based on the presence or absence of a normalization or stabilization in the level of one or more SAT biomarkers toward a predetermined standard level in the biological sample after the treatment.

16. The method of claim 3, wherein the presence or absence of a normalization or stabilization in the level of one or more SAT biomarkers toward a predetermined standard level after the treatment is predictive of the treatment efficacy of the sialic acid deficiency treatment.

17. The method of claim 4, wherein the presence or absence of a normalization or stabilization in the level of one or more SAT biomarkers toward a predetermined standard level after the treatment indicates whether a subject is suitable for the sialic acid deficiency treatment.

18. The method of claim 5, wherein the sialic acid deficiency treatment comprises at least one administration of a drug, and the treatment is continued with more administrations if there is a normalization or stabilization in the level of one or more SAT biomarkers toward a predetermined standard level after the initial trial test.

19. The method of claim 6, wherein the sialic acid deficiency treatment comprises at least one administration of a drug, and the treatment is continued with more administrations if there is a normalization or stabilization in the level of one or more SAT biomarkers toward a predetermined standard level after the initial trial test.

20. The method of claim 7, wherein the method comprises providing the information regarding the presence or absence of a normalization or stabilization in the level of one or more of the SAT biomarkers toward a predetermined standard level.

21. The method of claim 7 further comprising receiving the biological sample from the healthcare provider before the detecting step.

22. The method of claim 9, wherein the method comprises providing the level of one or more SAT biomarkers after at least one administration of the sialic acid deficiency treatment to an entity that provides a prediction or determination of the treatment efficacy based on the presence or absence of a normalization or stabilization in the level of one or more of the SAT biomarkers toward a predetermined standard level.

23. The method of any of claims 1-22, wherein the sialic acid deficiency is Hereditary Inclusion Body Myopathy (HIBM), Nonaka myopathy, or Distal Myopathy with Rimmed Vacuoles (DMRV).

24. The method of any of claims 1-22, wherein the sialic acid deficiency is Hereditary Inclusion Body Myopathy (HIBM).

25. The method of any of claims 1-22 wherein the sialic acid deficiency treatment is a sialic acid replacement therapy.

26. The method of 25 wherein the sialic acid replacement therapy is sialic acid or a derivative or analog thereof.

27. The method of any of claims 1-22 wherein the biological sample is selected from blood, skin, hair, hair follicles, saliva, oral mucous, vaginal mucous, sweat, tears, epithelial tissues, urine, semen, seminal fluid, seminal plasma, prostatic fluid, pre-ejaculatory fluid (Cowper's fluid), excreta, biopsy, ascites, cerebrospinal fluid, lymph, and tissue extract sample or biopsy sample.

28. The method of any of claims 1-22 wherein the biological sample is a blood sample.

29. The method of any of claims 1-22 wherein the one or more SAT biomarkers are selected from the biomarkers in Tables 2-13.

30. The method of any of claims 1-22 wherein the one or more SAT biomarkers are selected from AgRP, AR, BDNF, CD40-L, CgA, Cortisol, CK-MB, EGF, ENA-78, FGF-4, IGFBP-6, IL-3, IL-5, IL-7, IL-8, Kallikrein 5, LAP TGF-b1, M-CSF, MIP-3 alpha, MMP-1, MMP-3, MMP-9, MPO, Myoglobin, NT proBNP, NSE, Nr-CAM, PAI-1, PDGF-BB, S100-A4, S100-A6, ErbB3, SGOT, RANTES, Thrombospondin-1, TG, LGL, ENA78, and VEGF-C.

31. The method of any of claims 1-22 wherein the one or more SAT biomarkers are selected from BDNF, CD40-L, CgA, CK-MB, IGFBP-6, IL-3, IL-5, LAP TGF-b1, M-CSF, MMP-9, Myoglobin, NSE, PAI-1, PDGF-BB, ErbB3, RANTES, Thrombospondin-1, LGL, ENA78, and VEGF-C.

32. The method of any of claims 1-22 wherein the one or more SAT biomarkers are selected from BDNF, CD40-L, CgA, LAP TGF-b1, MMP-9, Myoglobin, PAI-1, PDGF-BB, ErbB3, RANTES, LGL, ENA78 and Thrombospondin-1.

33. The method of any of claims 1-22 wherein the one or more SAT biomarkers are selected from muscle inflammation and fibrosis biomarkers.

34. The method of any of claims 1-22 wherein the one or more SAT biomarkers are selected from ENA-78, CD40-L, IL-3, IL-5, IL-7, IL-8, LAP TGF-b1, M-CSF, MIP-3 alpha, Myeloperoxidase, RANTES, VEGF-C, AgRP, Thrombospondin-1, PDGF-BB, Matrix MMP-1, MMP-3 and MMP-9.

35. The method of any of claims 1-22 wherein the one or more SAT biomarkers are selected from CD40-L, IL-3, IL-5, IL-7, IL-8, LAP TGF-b1, RANTES, VEGF-C, AgRP, Thrombospondin-1, PDGF-BB and MMP-9.

36. The method of any of claims 1-22 wherein the one or more SAT biomarkers are selected from muscle and nerve biomarkers.

37. The method of any of claims 1-22 wherein the one or more SAT biomarkers are selected from BDNF, Cg-A, ErbB-3, NSE, Nr-CAM, EGF, FGF-4, Kallikrein 5 and PAI-1.

38. The method of any of claims 1-22 wherein the one or more SAT biomarkers are selected from BDNF, Cg-A, ErbB-3, NSE, FGF-4, and PAI-1.

39. The method of any of claims 1-22 wherein the one or more SAT biomarkers are selected from muscle damage biomarkers.

40. The method of any of claims 1-22 wherein the one or more SAT biomarkers are selected from SGOT, CK-MB and myoglobin.

41. The method of any of claims 1-22 wherein the one or more SAT biomarkers are selected from NT proBNP, S100-A4, S100-A6, IGFBP-6, Thyroglobulin, Amphiregulin and Cortisol.

42. The method of any of claims 1-41, comprising determining that the subject is suitable for sialic acid deficiency treatment based upon the presence or absence of a normalization or stabilization in one or more SAT biomarkers after at least one administration of the sialic acid deficiency treatment, optionally wherein the normalization or stabilization in said one or more SAT biomarkers has been maintained for at least about 1, 2, 3, 4, 5, 6, or 7 weeks or more prior to said determination.

43. The method of any of claims 1-41, comprising maintaining, increasing or reducing the dosage amount and/or frequency of the treatment upon the presence or absence of a normalization or stabilization in one or more SAT biomarkers after at least one administration of the sialic acid deficiency treatment, optionally wherein the normalization or stabilization in said one or more biomarkers has been maintained for at least about 1, 2, 3, 4, 5, 6, or 7 weeks or more prior to maintaining, increasing or reducing the dosage amount and/or frequency of the treatment.

44. A kit comprising reagents for detecting the level of one or more SAT biomarkers in a biological sample and an instruction for using the biomarker according to the method of any of claims 1-43.

45. A collection of level of a panel of SAT biomarkers comprising at least two or more SAT biomarkers selected from the biomarkers in Tables 2-13.

46. The collection of claim 45, wherein the one or more SAT biomarkers are selected from AgRP, AR, BDNF, CD40-L, CgA, Cortisol, CK-MB, EGF, ENA-78, FGF-4, IGFBP-6, IL-3, IL-5, IL-7, IL-8, Kallikrein 5, LAP TGF-b1, M-CSF, MIP-3 alpha, MMP-1, MMP-3, MMP-9, MPO, Myoglobin, NT proBNP, NSE, Nr-CAM, PAI-1, PDGF-BB, S100-A4, S100-A6, ErbB3, SGOT, RANTES, Thrombospondin-1, TG and VEGF-C.

47. The collection of claim 45, wherein the one or more SAT biomarkers are selected from BDNF, CD40-L, CgA, CK-MB, IGFBP-6, IL-3, IL-5, LAP TGF-b1, M-CSF, MMP-9, Myoglobin, NSE, PAI-1, PDGF-BB, ErbB3, RANTES, Thrombospondin-1 and VEGF-C.

48. The collection of claim 45, wherein the one or more SAT biomarkers are selected from CgA, LAP TGF-b1, MMP-9, Myoglobin, PAI-1, ErbB3, NCAM, LGL, ENA78, and M-CSF1.

49. An array comprising probes for detection of at least two SAT biomarkers, wherein the SAT biomarkers are selected from the biomarkers in Tables 2-13.

50. The array of claim 49, wherein the at least two SAT biomarkers are selected from AgRP, AR, BDNF, CD40-L, CgA, Cortisol, CK-MB, EGF, ENA-78, FGF-4, IGFBP-6, IL-3, IL-5, IL-7, IL-8, Kallikrein 5, LAP TGF-b1, M-CSF, MIP-3 alpha, MMP-1, MMP-3, MMP-9, MPO, Myoglobin, NT proBNP, NSE, Nr-CAM, PAI-1, PDGF-BB, S100-A4, S100-A6, ErbB3, SGOT, RANTES, Thrombospondin-1, TG and VEGF-C.

51. The array of claim 49, wherein the at least two SAT biomarkers are selected from BDNF, CD40-L, CgA, CK-MB, IGFBP-6, IL-3, IL-5, LAP TGF-b1, M-CSF, MMP-9, Myoglobin, NSE, PAI-1, PDGF-BB, ErbB3, RANTES, Thrombospondin-1 and VEGF-C.

52. The array of claim 49, wherein the at least two SAT biomarkers are selected from CgA, LAP TGF-b1, MMP-9, Myoglobin, PAI-1, ErbB3, NCAM, LGL, ENA78, and M-CSF1.

53. A combination of tests useful for predicting or determining the treatment efficacy of a sialic acid deficiency treatment comprising a first test for detecting the level of one SAT biomarker from a biological sample from a subject and a second test for detecting the level of a second SAT biomarker of said biological sample, wherein the first SAT biomarker is different from the second SAT biomarker.

54. The combination of tests of claim 53, wherein the SAT biomarkers are selected those in any of Tables 2-14.
